(19) United States
(12) Patent Application Publication
(10) Pub. No.: US 2022/0104866 A1
(43) Pub. Date: Apr. 7, 2022

(54) SYSTEMS AND METHODS FOR THERAPEUTIC NASAL TREATMENT

(71) Applicant: Neurent Medical Limited, Oranmore (IE)

(72) Inventors: David Townley, County Clare (IE); Cathal McLaughlin, Galway (IE); Norah O'Brien, Galway (IE)

(21) Appl. No.: 17/495,150

(22) Filed: Oct. 6, 2021

Related U.S. Application Data
(60) Provisional application No. 63/088,181, filed on Oct. 6, 2020.

(51) Int. Cl. A61B 18/14; A61B 18/12

(52) U.S. Cl. CPC A61B 18/14; A61B 2018/00327; A61B 18/1206

(57) ABSTRACT

The invention generally relates to systems and methods for providing detection, identification, and precision targeting of specific tissue(s) of interest in a nasal region of a patient for the treatment of a rhinosinusitis condition while minimizing or avoiding collateral damage to surrounding or adjacent non-targeted tissue, such as blood vessels, bone, and non-targeted neural tissue.

### CROSS-REFERENCE TO RELATED APPLICATIONS

This application claims priority to, and the benefit of, U.S. Provisional Application No. 63/088,181, filed Oct. 6, 2020, the content of which is incorporated by reference herein in its entirety.

### FIELD OF THE INVENTION

The invention generally relates to systems and methods for providing detection, identification, and precision targeting of specific tissue(s) of interest in a nasal region of a patient for the treatment of a rhinosinusitis condition while minimizing or avoiding collateral damage to surrounding or adjacent non-targeted tissue, such as blood vessels, bone, and non-targeted neural tissue.

### BACKGROUND

Rhinitis is an inflammatory disease of the nose and is reported to affect up to 40% of the population. It is the fifth most common chronic disease in the United States. The most common and impactful symptoms of rhinitis are congestion and rhinorrhea. Allergic rhinitis accounts for up to 65% of all rhinitis patients. Allergic rhinitis is an immune response to an exposure to allergens, such as airborne plant pollens, pet dander or dust. Non-allergic rhinitis is the occurrence of common rhinitis symptoms of congestion and rhinorrhea. As non-allergic rhinitis is not an immune response, its symptoms are not normally seasonal and are often more persistent. The symptoms of rhinitis include a runny nose, sneezing, and nasal itching and congestion.

Allergen avoidance and pharmacotherapy are relatively effective in the majority of mild cases, but these medications need to be taken on a long-term basis, incurring costs and side effects and often have suboptimal efficacy. For example, pharmaceutical agents prescribed for rhinosinusitis have limited efficacy and undesirable side effects, such as sedation, irritation, impairment to taste, sore throat, dry nose, and other side effects.

There are two modern surgical options: the delivery of thermal energy to the inflamed soft tissue, resulting in scarring and temporary volumetric reduction of the tissue to improve nasal airflow; and microdebrider resection of the inflamed soft tissue, resulting in the removal of tissue to improve nasal airflow. Both options address congestion as opposed to rhinorrhea and have risks ranging from bleeding and scarring to the use of general anesthetic. Importantly, these surgical procedures cannot precisely target neural tissue, thereby causing significant collateral damage to surrounding non-neural tissue (such as blood vessels) in order to treat rhinitis.

### SUMMARY

The invention recognizes that a problem with current surgical procedures is that such procedures are not accurate and cause significant collateral damage. In particular, the invention recognizes that knowing certain properties of tissue, both active and passive, at a given target site prior to, and during electrotherapeutic treatment (i.e., neuromodulation, ablation, etc.), provides an ability to more precisely target a specific tissue of interest (i.e., targeted tissue) and minimize and/or prevent collateral damage to adjacent or surrounding non-targeted tissue.

For example, certain target sites intended to undergo treatment may consist of more than one type of tissue (i.e., nerves, muscles, bone, blood vessels, etc.). In particular, a tissue of interest (i.e., the specific tissue to undergo treatment) may be adjacent to one or more tissues that are not of interest (i.e., tissue that is not intended to undergo treatment). In one scenario, a surgeon may wish to provide electrotherapeutic stimulation to a nerve tissue, while avoiding providing any such stimulation to an adjacent blood vessel, for example, as unintended collateral damage may result in damage to the blood vessel and cause further complications. In such a scenario, the specific type of targeted tissue may generally dictate the level of electrical stimulation required to elicit a desired effect. Furthermore, physical properties of the targeted tissue, including the specific location and depth of the targeted tissue, in relation to the non-targeted tissue, further impacts the level of electrical stimulation necessary to result in effective therapeutic treatment.

The invention solves these problems by providing a treatment device and a console unit for providing intuitive and automated control and targeting of energy output from the treatment device sufficient to ensure successful treatment of a condition, such as a nasal condition, including rhinosinusitis. In particular, the console unit provides a user, via an interactive interface, with comprehensive operational instructions for performing a given procedure and, in response to user input, further provides automatic and precise control over the ablation/modulation of the targeted tissue while minimizing and/or preventing collateral damage to surrounding or adjacent non-targeted tissue at the target site. More specifically, the console unit provides the user with step-by-step guidance, in the form of selectable inputs, for treating, via the treatment device, rhinosinusitis. It should be noted, however, that the systems and methods of the present invention can be used to treat various conditions, and is not limited to the treatment of a nasal condition.

Such step-by-step guidance provided via the interactive interface of the console unit may include, for example, directing the user through the initial set up of the device with the console unit, including authenticating the device (to ensure that the device is in fact suitable and/or authorized to operate with the console unit), and, upon authenticating the device, further directing the user to select a location in which to provide treatment (i.e., left or right nasal cavity). Based on the user's selection of a given nasal cavity, the console unit further provides the user with an indication as to when the device is primed and ready to perform treatment in the selected location. In particular, the console unit is configured to perform an assessment of one or more electrodes associated with an end effector of the treatment device, wherein such assessment includes a determination of whether electrodes are available for use (i.e., via an impedance assessment of each electrode).

Depending on the availability of one or more electrodes for energy delivery, the user may be presented with operational inputs, including the option of initiating treatment. Upon receiving user selection of treatment initiation, the console unit is configured to determine a specific treatment pattern for controlling delivery of energy at a specific level for a specific period of time to the tissue of interest (i.e., the targeted tissue) sufficient to ensure successful ablation/modulation of the targeted tissue while minimizing and/or preventing collateral damage to surrounding or adjacent non-targeted tissue at the target site. More specifically, the console unit has the ability to characterize, prior to a therapeutic treatment, the type of tissue at a target site by sensing at least bioelectric properties of tissue, wherein such characterization includes identifying specific types of tissue present at the target site. For example, different tissue types include different physiological and histological characteristics. As a result of the different characteristics, different tissue types have different associated bioelectrical properties and thus exhibit different behavior in response to application of energy applied thereto. By knowing such properties of a given tissue, the systems and methods are configured to determine a specific treatment pattern for controlling the delivery of energy. In particular, a given treatment pattern may include, for example, a predetermined treatment time, a precise level of energy to be delivered, and a predetermined impedance threshold for that particular tissue.

The console unit is further configured to receive and process real-time feedback data associated with the targeted tissue undergoing treatment and further provide, via the interactive interface, information to the user, specifically related to the ongoing operation of the treatment device as well as a status of the therapy during the procedure, including indications as to whether treatment via respective electrodes is successful (i.e., complete) or unsuccessful (i.e., incomplete). The console unit is further configured to process the feedback data to further ensure that energy delivered is maintained within the scope of the treatment pattern. More specifically, the console unit is configured to automatically control delivery of energy to the targeted tissue based on the processing of the real-time feedback data, wherein such data includes at least impedance measurement data associated with the targeted tissue collected during delivery of energy to the targeted tissue. The controller is configured to process impedance measurement data to detect a slope change event (e.g., an asymptotic rise) within an impedance profile associated with the treatment, wherein, with reference to the predetermined impedance threshold, the slope change event is indicative of whether the ablation/modulation of the targeted tissue is successful. In turn, the controller is configured to automatically control the delivery of energy to the targeted tissue based on real-time monitoring of feedback data, most notably impedance data, to ensure the desired ablation/modulation is achieved. As a result, the console unit is able to ensure that optimal energy is delivered in order to delay the onset of impedance roll-off, until the target ablation/modulation depth is achieved, while maintaining clinically relevant treatment time. Accordingly, the invention solves the problem of causing unnecessary collateral damage to non-targeted tissue during a procedure involving the application of electrotherapeutic stimulation at a target site composed of a variety of tissue types.

Following the delivery of energy from one or more electrodes, resulting in either successful or unsuccessful treatment of respective targeted tissue, the console unit performs post-treatment analysis. The post-treatment analysis includes a determination of any prior treatments performed, including prior use of the electrodes on prior targeted tissue for a given nasal cavity, a status of such prior use, including whether such treatment was successful or unsuccessful, and a determination of any and all further treatments to be performed. In turn, the console unit provides, via the interactive interface, one or more post-procedure inputs from which the user may select for controlling subsequent use of the treatment device to ensure that the overall procedure (i.e., treatment of rhinosinusitis) is completed by ensuring that all portions of targeted tissue undergo treatment.

Accordingly, the systems and methods of the present invention provide an intuitive, user-friendly, and semi-automated means of treating rhinosinusitis conditions, including precise and focused application of energy to the intended targeted tissue without causing collateral and unintended damage or disruption to other tissue and/or structures. Thus, the efficacy of a vidian neurectomy procedure can be achieved with the systems and methods of the present invention without the drawbacks discussed above. Most notably, the console unit provides a user (i.e., surgeon or other medical professional) with relatively simple operational instructions, in the form of step-by-step guidance via an interactive interface, for performing the procedure, such as directing the user to select a specific nasal cavity to treat, providing indications (both visual and audible) as to when the treatment device is ready to perform a given treatment, providing automated control over the delivery of energy to the targeted tissue upon user-selected input to initiate treatment, and further providing a status of therapy during the procedure and after the procedure, including indications (e.g., visual and/or audible) as to whether the treatment is successful or unsuccessful. Accordingly, such treatment is effective at treating rhinosinusitis conditions while greatly reducing the risk of causing lateral damage or disruption to other tissue or structures (i.e., non-targeted tissue, such as blood vessels, bone, and non-targeted neural tissue), thereby reducing the likelihood of unintended complications and side effects.

One aspect of the present invention provides a system for treating a condition within a sino-nasal cavity of a patient. The system includes a console unit configured to be operably associated with a treatment device and control operation thereof. The console unit is configured to analyze identifying data associated with a treatment device upon connection of the treatment device to the console unit, determine authenticity of the treatment device based on the analysis of the identifying data, and output, via an interactive interface associated with the console unit, an alert to a user indicating at least the authenticity determination. The alert may include, for example, at least one of audible alert and visual alert indicating the incompatibility of the treatment device. For example, the alert may include at least one of text and a color coding displayed on a graphical user interface (GUI) indicating the incompatibility of the treatment device and further provide one or more suggested actions. The one or more suggested actions may include a suggestion that the user couple an authentic and compatible or valid treatment device to the console unit.

In some embodiments, the analysis of the identifying data comprises correlating the identifying data with authentication data. The authentication data may include a unique identifier including an authentication key or identity number associated with authentic treatment devices permitted to be used with the console unit. The treatment device is determined to be authentic upon a positive correlation and determined to be inauthentic upon a negative correlation. The console unit permits transmission of energy from an energy source to an energy delivery element of the treatment device in response to a positive correlation and prevents transmission of energy from an energy source to an energy delivery element of the treatment device in response to a negative correlation. In some embodiments, the energy includes radiofrequency (RF) energy from an RF generator and the energy delivery element of the treatment device comprises one or more electrodes. The one or more electrodes are provided on one or more respective portions of an end effector of the treatment device.

Upon a determination that the treatment device is inauthentic, the console unit is configured to output at least one of audible alert and visual alert indicating to the user that the treatment device in inauthentic and incompatible or invalid with the console unit and further prevent transmission of energy from an energy source to an energy delivery element of the treatment device in response to a negative correlation.

Upon a positive correlation and determination that the treatment device is authentic, the console unit is further configured to determine any prior use of the treatment device, including whether such prior use was associated with the console unit or a different console unit, based on the analysis of the identifying data. Upon a determination that the treatment device is unused, the console unit is configured to set a use count of the treatment device to default count and further output, via the interactive interface, an alert to the user indicating that the treatment device is set for use and further permit transmission of energy from an energy source to an energy delivery element of the treatment device.

Upon a determination that the treatment device has prior use and such prior use was associated with a different console unit, the console unit is configured to output at least one of audible alert and visual alert indicating to the user that the treatment device is incompatible with the console unit and further prevent transmission of energy from an energy source to an energy delivery element of the treatment device. The alert may include at least one of text and a color coding displayed on a graphical user interface (GUI) indicating the incompatibility of the treatment device and further providing one or more suggested actions. The one or more suggested actions may include a suggestion that the user couple an authentic and compatible treatment device to the console unit.

Upon a determination that the treatment device has prior use and such prior use was associated with the console unit, the console unit is configured to determine an amount and/or timeframe of the prior use, based on the analysis of the identifying data. Upon a determination that the prior use was within a predetermined grace period, the console unit is configured to output, via the interactive interface, an alert to the user indicating that the treatment device is set for use and further permit transmission of energy from an energy source to an energy delivery element of the treatment device. Upon a determination that the prior use with outside of a predetermined grace period, the console unit is configured to output, via the interactive interface, at least one of audible alert and visual alert indicating to the user that the treatment device is expired and further prevent transmission of energy from an energy source to an energy delivery element of the treatment device.

Another aspect of the present invention provides a system for treating a condition within a sino-nasal cavity of a patient. The system includes a treatment device including an end effector comprising one or more electrodes for delivering energy to one or more target sites within the sino-nasal cavity of the patient. The system further includes a console unit operably associated with the treatment device. The console unit is configured to receive, via user input with an interactive interface associated with the console unit, a request for a determination of availability of the one or more electrodes for applying treatment to one or more target sites within a selected one of a left side and a right side of the sino-nasal cavity of the patient and initiate, in response to the request, an impedance assessment of the one or more electrodes within the selected one of the left and right sides of the sino-nasal cavity. The console unit is further configured to output, via the interactive interface, an alert to a user indicating a determined availability of the one or electrodes based on the impedance assessment.

Upon initiating the impedance assessment, the console unit is configured to receive, from the one or more electrodes, impedance measurement data associated with tissue at the one or more target sites within the selected one of the left and right sides of the sino-nasal cavity, and process the impedance measurement data to calculate a baseline impedance value for each of the one or more electrodes.

The processing of the impedance measurement data may include calculating aggregate impedance values for each of the one or more electrodes or across a set of multiple pairs of the electrodes within a selected one of the left and right sides of the sino-nasal cavity. In some embodiments, the console unit is configured to process impedance measurement data of all pairs of electrodes of the set within the selected one of the left and right sides of the sino-nasal cavity. In some embodiments, the determined availability of the one or more pairs of the electrodes is based on a comparison of the calculated baseline impedance value with a predetermined range of baseline impedance values. The predetermined range of baseline impedance values includes a low baseline impedance value of approximately 100 ohms and a high baseline impedance value of approximately 1 kohms. In some embodiments, the predetermined range of baseline impedance values includes a low baseline impedance value of approximately 400 ohms and a high baseline impedance value of approximately 700 ohms.

In some embodiments, the end effector is multi-segmented and comprises a plurality of support structures that each comprises one or more electrodes. In some embodiments, at least one of a single, a pair, and a multitude of the plurality of support structures is determined to be available for applying treatment, via one or more associated electrodes, to one or more target sites when the calculated baseline value falls within the predetermined range of baseline impedance values. In some embodiments, at least one of a single, a pair, and a multitude of the plurality of support structures is determined to be unavailable for applying treatment, via one or more associated electrodes, to one or more target sites when the calculated baseline value falls outside the predetermined range of baseline impedance values.

In some embodiments, the console unit is configured to permit repositioning of the at least one of the single, the pair, and the multitude of the plurality of support structures determined to be unavailable for applying treatment, via one or more associated electrodes, to one or more target sites when the calculated baseline value falls outside the predetermined range of baseline impedance values. In turn, the console unit is configured to output at least one of audible alert and visual alert, via the interactive interface, indicating to the user the availability treatment device to provide treatment once successfully repositioned based on a comparison of the calculated baseline impedance value with a predetermined range of baseline impedance values. The visual alert comprises at least one of text and a first color coding displayed on a graphical user interface (GUI).

In some embodiments, the console unit is configured to permit transmission of energy from an energy source to one or more electrodes associated with the at least one of the single, the pair, and the multitude of the plurality of support structures determined to be available. In some embodiments, the console unit is configured to prevent transmission of energy from an energy source to one or more electrodes associated with the at least one of the single, the pair, and the multitude of the plurality of support structures determined to be unavailable. The energy may include radiofrequency (RF) energy from an RF generator.

Upon a determination that at least a minimum required number of pairs of electrodes associated with the at least one of the single, the pair, and the multitude of the plurality of support structures are available, the console unit is configured to output at least one of audible alert and visual alert, via the interactive interface, indicating to the user that the treatment device is ready to provide treatment and further permit transmission of energy from an energy source to one or more electrodes for subsequent delivery of energy to one or more target sites within the selected one of the left and right sides of the sino-nasal cavity. The visual alert may include at least one of text and a first color coding displayed on a graphical user interface (GUI) indicating the availability of one or more pairs of electrodes associated with the at least one of the single, the pair, and the multitude of the plurality of support structures.

Upon a determination that one or more pairs of electrodes associated with the at least one of the single, the pair, and the multitude of the plurality of support structures is unavailable, the console unit is configured to output at least one of audible alert and visual alert, via the interactive interface, indicating to the user that the treatment device not ready to provide treatment and further prevent transmission of energy from an energy source to one or more electrodes to thereby prevent subsequent delivery of energy to one or more target sites within the selected one of the left and right sides of the sino-nasal cavity. Again, the visual alert may include at least one of text and a second color coding displayed on a graphical user interface (GUI) indicating the unavailability of one or more of the plurality of support structures.

The multi-segmented end effector may include a proximal segment that is spaced apart from a distal segment, wherein each of the proximal and distal segments comprises a plurality of support structures that each comprises one or more electrodes. At least one of the plurality of support structures comprises a first support structure from the proximal segment and a second support structure from the distal segment. The electrodes associated with the at least one of the plurality of support structures may be configured to deliver energy to the one or more target sites within the selected one of the left and right sides of the sino-nasal cavity of the patient to disrupt multiple neural signals to, and/or result in local hypoxia of, mucus producing and/or mucosal engorgement elements, thereby reducing production of mucus and/or mucosal engorgement within a nose of the patient and reducing or eliminate one or more symptoms associated with at least one of rhinitis, congestion, and rhinorrhea.

Accordingly, the targeted tissue may be associated with one or more target sites proximate or inferior to a sphenopalatine foramen, wherein energy is delivered at a level sufficient to therapeutically modulate postganglionic parasympathetic nerves innervating nasal mucosa at foramina and/or microforamina of a palatine bone of the patient and causes multiple points of interruption of neural branches extending through foramina and/or microforamina of palatine bone. Additionally, or alternatively, the targeted tissue may be associated with one or more target sites proximate or inferior to a sphenopalatine foramen, wherein energy is delivered at a level sufficient to ablate targeted tissue to thereby cause thrombus formation within one or more blood vessels associated with mucus producing and/or mucosal engorgement elements within the nose, wherein the resulting local hypoxia of the mucus producing and/or mucosal engorgement elements results in decreased mucosal engorgement to thereby increase volumetric flow through a nasal passage of the patient.

Another aspect of the present invention provides a system for treating a condition within a sino-nasal cavity of a patient. The system includes a treatment device including a multi-segment end effector comprising a plurality of sets of support structures, wherein each set comprises one or more support structures and each support structure comprises one or more electrodes for delivering energy to one or more target sites within the sino-nasal cavity of the patient. The system further includes a console unit operably associated with the treatment device. The console unit is configured to receive, via user input with an interactive interface associated with the console unit, a request to initiate treatment of a selected one of a left side and a right side of the sino-nasal cavity of the patient and identify, in response to the request, one or more sets of support structures to be activated for treating the selected one of the left and right side of the sino-nasal cavity. The console unit is further configured to calculate a treatment pattern for controlling delivery of energy from electrodes associated with at least one of a single, a pair, and a multitude of the plurality of support structures of a given identified set, receive feedback data associated with each of the plurality of support structures upon supplying treatment energy to respective electrodes, and process the feedback data to determine a status of each of the plurality of support structures with respect to the treatment pattern. The status may generally include an incomplete state, a successful state, and an unsuccessful state.

The treatment pattern may include at least one of a predetermined treatment time, a level of energy to be delivered from the electrodes, and a predetermined impedance threshold. The feedback data may include impedance measurement data associated with tissue at the one or more target sites within the selected one of the left and right sides of the sino-nasal cavity. The console unit is configured to process the impedance measurement data to calculate at least one of a baseline impedance value prior to delivery of energy from electrodes to the tissue for the determination of whether at least one of a single, a pair, and a multitude of the plurality of support structures is available, and an active impedance value during delivery of energy from electrodes of an available one of the at least one of the single, pair, and multitude of the plurality of support structures to the tissue. In turn, the console unit is further configured to determine availability of each of the at least one of the single, pair, and multitude of the plurality of support structures for a given set based on a comparison of the calculated baseline impedance value with a predetermined range of baseline impedance values. At least one support structure is determined to be available for applying treatment when the calculated baseline value falls within the predetermined range of baseline impedance values and unavailable for applying treatment when the calculated baseline value falls outside the predetermined range of baseline impedance values.

The feedback data may further include an elapsed time of delivery of energy from electrodes of an available one of the at least one of the single, pair, and multitude of the plurality of support structures to the tissue. The console unit is configured to compare the elapsed time with the predetermined treatment time to determine a status of the at least one of the single, pair, and multitude of the plurality of support structures. The console unit determines one or more support structures to be in a successful state when the elapsed time of delivery of energy exceeds the predetermined treatment time, all available support structures of a given set have delivered treatment, and no incomplete support structures of that given set are present. The console unit determines one or more support structures to be in an unsuccessful state, and disables energy delivery from electrodes associated with the one or more support structures, when the elapsed time of delivery of energy exceeds the predetermined treatment time, all available support structures of a given set have delivered treatment, and the one or more support structures remain currently active and incomplete upon the elapsed time exceeding the predetermined treatment time by greater than or equal to three seconds.

If the elapsed time is less than the predetermined treatment time, the console unit is configured to process the active impedance value to determine a status of one or more support structures. The processing of the active impedance value comprises using an algorithm to determine whether the one or more support structures is in at least one of a successful state or an unsuccessful state based on a comparison of the active impedance value with at least one of a predetermined minimum impedance value, a predetermined low terminal impedance value, and a predetermined high terminal impedance value. If the active impedance value is less than the predetermined minimum impedance value, the console unit determines the one or more support structures to be in an unsuccessful state and disables energy delivery from electrodes associated with the one or more support structures.

If the active impedance value is greater than the predetermined minimum impedance value and greater than the predetermined low terminal impedance value, the console unit is configured to calculate a slope change for the detection of a slope event. Upon detecting a slope event, the console unit determines that the at least one of the single, pair, and multitude of the plurality of support structures to be in a successful state if a negative slope event is detected and disables energy delivery from electrodes associated with the support structures and further determines the at least one of the single, pair, and multitude of the plurality of support structures to be in an unsuccessful state if a negative slope event is not detected and disables energy delivery from electrodes associated with the support structures.

In the absence of detecting a slope event, the console unit determines the at least one of the single, pair, and multitude of the plurality of support structures to be in an in an unsuccessful state if the active impedance value is greater than the predetermined high terminal impedance value and disables energy delivery from electrodes associated with the at least one of the single, pair, multitude of the plurality of support structures.

The console unit is further configured to output, via the interactive interface, an alert to a user indicating a status of each of the at least one of the single, pair, and multitude of the plurality of support structures. For example, the console unit is configured to output at least a visual alert indicating a status of each of the at least one of the single, pair, and multitude of the plurality of support structures of a given set. The visual alert may include at least one of a color and text displayed on a graphical user interface (GUI) and indicating each of the incomplete state, successful state, and unsuccessful state.

Another aspect of the present invention provides a system for treating a condition within a sino-nasal cavity of a patient. The system includes a treatment device including a multi-segment end effector comprising a plurality of sets of support structures, wherein each set comprises at least one of a single, pair, and multitude of a plurality of support structures and each support structure comprises one or more electrodes for delivering energy to one or more target sites within the sino-nasal cavity of the patient for treatment of a condition thereof. The system further includes a console unit operably associated with the treatment device and including a database for storing treatment data associated with prior use of the end effector in delivering energy to at least one of a left side and a right side of the sino-nasal cavity of the patient. The console unit is configured to provide, via an interactive interface associated with the console unit, one or more post-procedure inputs for controlling subsequent use of the end effector based on an analysis of the treatment data, receive, via user input with the interactive interface, a selected one of the post-procedure inputs, and initiate, in response to the selected post-procedure input, one or more actions controlling delivery of energy to one or more target sites within at least one of the left and right sides of the sino-nasal cavity.

The one or more post-procedure inputs may include initiating one or more additional applications of treatment to a selected one of the left and right sides of the sino-nasal cavity having already undergone treatment, initiating application of treatment to an untreated one of the left and right sides of the sino-nasal cavity, or confirming completion of entire procedure.

The treatment data may include data associated with prior use of one or more electrodes in delivering energy to one or more associated target sites within either of the left and rights sides of the sino-nasal cavity and an indication of whether treatment applied, via the delivery of energy, is complete for either of the left and right sides of the sino-nasal cavity. In the event that treatment of only one of left and right sides of the sino-nasal cavity is complete, the console unit is configured to provide, via the interactive interface, the post-procedure inputs.

Upon receipt of a user selected request for one or more additional applications of treatment to be applied to the left or right side of the sino-nasal cavity having already undergone treatment, the console unit is configured to initiate an impedance assessment of the at least one of the single, pair, and multitude of the plurality of support structures of a given set associated with the already treated left or right side of the sino-nasal cavity and determine availability of each of the at least one of the single, pair, and multitude of the plurality of support structures for applying treatment, via delivery of energy from one or more associated electrodes, to one or more target sites within the already treated left or right side of the sino-nasal cavity.

The console unit is configured to calculate a treatment pattern for controlling delivery of energy from electrodes associated with each of the at least one of the single, pair, and multitude of the plurality of support structures of the given set determined to be available, receive feedback data associated with each of the at least one of the single, pair, and multitude of the plurality of support structures upon supplying treatment energy to respective electrodes, and process the feedback data to determine a status of each of the at least one of the single, pair, and multitude of the plurality of support structures with respect to the treatment pattern, wherein the status comprises an incomplete state, a successful state, and an unsuccessful state. The treatment pattern may include at least one of a predetermined treatment time, a level of energy to be delivered from the electrodes, and a predetermined impedance threshold. Accordingly, the feedback data may include impedance measurement data associated with tissue at the one or more target sites within the already treated left or right side of the sino-nasal cavity and an elapsed time of delivery of energy from electrodes of an available one of the at least one of the single, pair, and multitude of the plurality of support structures to the tissue.

The console unit is configured to process the impedance measurement data to calculate at least an active impedance value during delivery of energy from electrodes of an available one of the at least one of the single, pair, and multitude of the plurality of support structures to the tissue. The console unit is configured to compare the elapsed time with the predetermined treatment time to determine a status of the at least one of the single, pair, and multitude of the plurality of support structures.

The console unit determines at least one of the single, pair, and multitude of the plurality of support structures to be in a successful state when the elapsed time of delivery of energy exceeds the predetermined treatment time, all available support structures of a given set have delivered treatment, and no incomplete pairs of support structures of that given set are present. The console unit determines at least one of the single, pair, and multitude of the plurality of support structures to be in an unsuccessful state, and disables energy delivery from electrodes associated with the at least one of the single, pair, and multitude of the plurality of support structures, when the elapsed time of delivery of energy exceeds the predetermined treatment time, all available support structures of a given set have delivered treatment, and the at least one of the single, pair, and multitude of the plurality of support structures remains currently active and incomplete upon the elapsed time exceeding the predetermined treatment time by greater than or equal to three seconds. If the elapsed time is less than the predetermined treatment time, the console unit is configured to process the active impedance value to determine a status of the at least one of the single, pair, and multitude of the plurality of support structures.

The processing of the active impedance value comprises using an algorithm to determine whether the at least one of the single, pair, and multitude of the plurality of support structures is in at least one of a successful state or an unsuccessful state based on a comparison of the active impedance value with at least one of a predetermined minimum impedance value, a predetermined low terminal impedance value, and a predetermined high terminal impedance value. The console unit determines the at least one of the single, pair, and multitude of the plurality of support structures to be in an unsuccessful state if the active impedance value is less than the predetermined minimum impedance value and disables energy delivery from electrodes associated with the at least one of the single, pair, and multitude of the plurality of support structures. If the active impedance value is greater than the predetermined minimum impedance value and greater than the predetermined low terminal impedance value, the console unit is configured to calculate a slope change for the detection of a slope event. Upon detecting a slope event, the console unit determines the at least one of the single, pair, and multitude of the plurality of support structures to be in a successful state if a negative slope event is detected and disables energy delivery from electrodes associated with the at least one of the single, pair, and multitude of the plurality of support structures, and further determines the at least one of the single, pair, and multitude of the plurality of support structures to be in an unsuccessful state if a negative slope event is not detected and disables energy delivery from electrodes associated with the pair of support structures. In the absence of detecting a slope event, the console unit determines the at least one of the single, pair, and multitude of the plurality of support structures to be in an in an unsuccessful state if the active impedance value is greater than the predetermined high terminal impedance value and disables energy delivery from electrodes associated with the at least one of the single, pair, and multitude of the plurality of support structures.

The console unit is further configured to output, via the interactive interface, at least a visual alert indicating a status of each of the at least one of the single, pair, and multitude of the plurality of support structures of the given set. The visual alert includes at least one of a color and text displayed on a graphical user interface (GUI) and indicating each of the incomplete state, successful state, and unsuccessful state.

Upon receipt of a user selected request for initiating application of treatment to an untreated one of the left and right sides of the sino-nasal cavity, the console unit is configured to initiate an impedance assessment of at least one of the single, pair, and multitude of the plurality of support structures of a given set associated with the untreated one of the left and right sides of the sino-nasal cavity, and determine availability of each of the at least one of the single, pair, and multitude of the plurality of support structures for applying treatment, via delivery of energy from one or more associated electrodes, to one or more target sites within the treated left or right side of the sino-nasal cavity. The console unit is configured to calculate a treatment pattern for controlling delivery of energy from electrodes associated with each of the at least one of the single, pair, and multitude of the plurality of support structures of the given set determined to be available, receive feedback data associated with each of the at least one of the single, pair, and multitude of the plurality of support structures upon supplying treatment energy to respective electrodes, and process the feedback data to determine a status of each of the at least one of the single, pair, and multitude of the plurality of support structures with respect to the treatment pattern, wherein the status comprises an incomplete state, a successful state, and an unsuccessful state. The console unit is further configured to output, via the interactive interface, at least a visual alert indicating a status of each pair of support structures of the given set. The visual alert includes at least one of a color and text displayed on a graphical user interface (GUI) and indicating each of the incomplete state, successful state, and unsuccessful state.

The treatment pattern includes at least one of a predetermined treatment time, a level of energy to be delivered from the electrodes, and a predetermined impedance threshold. The feedback data includes impedance measurement data associated with tissue at the one or more target sites within the already treated left or right side of the sino-nasal cavity and an elapsed time of delivery of energy from electrodes of an available one of the at least one of the single, pair, and multitude of the plurality of support structures to the tissue. The console unit is configured to process the impedance measurement data to calculate at least an active impedance value during delivery of energy from electrodes of an available one of the at least one of the single, pair, and multitude of the plurality of support structures to the tissue. The console unit is configured to compare the elapsed time with the predetermined treatment time to determine a status of the at least one of the single, pair, and multitude of the plurality of support structures. The console unit determines a pair of support structures to be in a successful state when the elapsed time of delivery of energy exceeds the predetermined treatment time, all available support structures of a given set have delivered treatment, and no incomplete support structures of that given set are present. The console unit determines at least one of the single, pair, and multitude of the plurality of support structures to be in an unsuccessful state, and disables energy delivery from electrodes associated with the at least one of the single, pair, and multitude of the plurality of support structures, when the elapsed time of delivery of energy exceeds the predetermined treatment time, all available support structures of a given set have delivered treatment, and the pair of support structures remains currently active and incomplete upon the elapsed time exceeding the predetermined treatment time by greater than or equal to three seconds.

If the elapsed time is less than the predetermined treatment time, the console unit is configured to process the active impedance value to determine a status of the at least one of the single, pair, and multitude of the plurality of support structures. The processing of the active impedance value comprises using an algorithm to determine whether the at least one of the single, pair, and multitude of the plurality of support structures is in at least one of a successful state or an unsuccessful state based on a comparison of the active impedance value with at least one of a predetermined minimum impedance value, a predetermined low terminal impedance value, and a predetermined high terminal impedance value. The console unit determines the at least one of the single, pair, and multitude of the plurality of support structures to be in an unsuccessful state if the active impedance value is less than the predetermined minimum impedance value and disables energy delivery from electrodes associated with the at least one of the single, pair, and multitude of the plurality of support structures. If the active impedance value is greater than the predetermined minimum impedance value and greater than the predetermined low terminal impedance value, the console unit is configured to calculate a slope change for the detection of a slope event. Upon detecting a slope event, the console unit determines the at least one of the single, pair, and multitude of the plurality of support structures to be in a successful state if a negative slope event is detected and disables energy delivery from electrodes associated with the at least one of the single, pair, and multitude of the plurality of support structures and determines the at least one of the single, pair, and multitude of the plurality of support structures to be in an unsuccessful state if a negative slope event is not detected and disables energy delivery from electrodes associated with the at least one of the single, pair, and multitude of the plurality of support structures. In the absence of detecting a slope event, the console unit determines the at least one of the single, pair, and multitude of the plurality of support structures to be in an in an unsuccessful state if the active impedance value is greater than the predetermined high terminal impedance value and disables energy delivery from electrodes associated with the at least one of the single, pair, and multitude of the plurality of support structures.

### BRIEF DESCRIPTION OF THE DRAWINGS

FIGS. 1A and 1B are diagrammatic illustrations of a system for treating a condition of a patient using a handheld device according to some embodiments of the present disclosure.

FIG. 2 is a diagrammatic illustration of the console coupled to the handheld device consistent with the present disclosure, further illustrating one embodiment of an end effector of the handheld device for delivering energy to tissue at one or more target sites.

FIG. 3 is a side view of one embodiment of a handheld device for providing therapeutic treatment consistent with the present disclosure.

FIG. 4 is an enlarged, perspective view of one embodiment of an end effector consistent with the present disclosure.

FIGS. 5A-5F are various views of the multi-segment end effector consistent with the present disclosure.

FIG. 5A is an enlarged, perspective view of the multi-segment end effector illustrating the first (proximal) segment and second (distal) segment. FIG. 5B is an exploded, perspective view of the multi-segment end effector. FIG. 5C is an enlarged, top view of the multi-segment end effector. FIG. 5D is an enlarged, side view of the multi-segment end effector. FIG. 5E is an enlarged, front (proximal facing) view of the first (proximal) segment of the multi-segment end effector. FIG. 5F is an enlarged, front (proximal facing) view of the second (distal) segment of the multi-segment end effector.

FIG. 6 is a perspective view, partly in section, of a portion of a support element illustrating an exposed conductive wire serving as an energy delivery element or electrode element.

FIG. 7 is a cross-sectional view of a portion of the shaft of the handheld device taken along lines 7-7 of FIG. 3.

FIG. 8A is a side view of the handle of the handheld device.

FIG. 8B is a side view of the handle illustrating internal components enclosed within.

FIG. 9 is a block diagram illustrating the console unit of the present disclosure and authentication of a handheld treatment device to be used with the console unit.

FIG. 10 is a block diagram illustrating authentication of the treatment device in greater detail.

FIG. 11 is a block diagram illustrating an availability assessment of one or more electrodes of an end effector of a handheld treatment device of the present disclosure.

FIG. 12 is a block diagram illustrating the availability assessment in greater detail.

FIG. 13 is a block diagram illustrating controlled and targeted energy delivery from one or more electrodes of an end effector of the treatment device via the console unit based on a calculated treatment pattern.

FIG. 14A is a block diagram illustrating delivery of non-therapeutic energy from electrodes of the end effector at a frequency/waveform for sensing one or more properties associated with one or more tissues at a target site in response to the non-therapeutic energy.

FIG. 14B is a block diagram illustrating communication of sensor data from the handheld device to the controller and subsequent determination, via the controller, of a treatment pattern for controlling energy delivery based on the sensor data for precision targeting of tissue of interest and to be treated.

FIG. 14C is a block diagram illustrating delivery of energy to the target site based on the treatment pattern output from the controller, monitoring of real-time feedback data associated with the targeted tissue undergoing treatment, and subsequent control over the delivery of energy based on the processing of the feedback data.

FIGS. 15A and 15B are graphs illustrating impedance profiles of two different sets of electrodes delivering energy to respective portions of targeted tissue, wherein the graphs illustrate a slope change event (e.g., asymptotic rise) which is indicative of whether the ablation/modulation of the targeted tissue is successful.

FIGS. 16A and 16B are block diagrams illustrating post-treatment analysis, including post-procedure inputs provided by the console from which a user may select for controlling subsequent use of the treatment device to ensure that the overall procedure is completed.

FIG. 17 is a flow diagram illustrating one embodiment of a method for authenticating a handheld treatment device to be used with the console unit of the present disclosure.

FIGS. 18A-18C show a continuous flow diagram illustrating a method for providing an availability assessment of one or more electrodes of an end effector of a handheld device and subsequently providing an indication (i.e., visual and/or audible alert(s)) as to whether the device is primed and ready to perform treatment in the selected location.

FIGS. 19A-19E show a continuous flow diagram illustrating a method for targeted energy delivery to a targeted tissue based, at least in part, on a treatment pattern output from the controller, monitoring of real-time feedback data associated with the targeted tissue undergoing treatment, and subsequent control over the delivery of energy based on the processing of the feedback data.

FIGS. 20A-20D show a continuous flow diagram illustrating a method for post-treatment analysis.

### DETAILED DESCRIPTION

The invention recognizes that a problem with current surgical procedures is that such procedures are not accurate and cause significant collateral damage. In particular, the invention recognizes that knowing certain properties of tissue, both active and passive, at a given target site prior to, and during electrotherapeutic treatment (i.e., neuromodulation, ablation, etc.), provides an ability to more precisely target a specific tissue of interest (i.e., targeted tissue) and minimize and/or prevent collateral damage to adjacent or surrounding non-targeted tissue.

Neuromodulation, for example, is technology that acts directly upon nerves. It is the alteration, or modulation, of nerve activity by delivering electrical or pharmaceutical agents directly to a target area. Neuromodulation devices and treatments have been shown to be highly effective at treating a variety of conditions and disorders. The most common indication for neuromodulation is treatment of chronic pain. However, the number of neuromodulation applications over the years has increased to include more than just the treatment of chronic pain, such as deep brain stimulation (DBS) treatment for Parkinson's disease, sacral nerve stimulation for pelvic disorders and incontinence, and spinal cord stimulation for ischemic disorders (angina, peripheral vascular disease).

Neuromodulation is particularly useful in the treatment of peripheral neurological disorders. There are currently over 100 kinds of peripheral nerve disorders, which can affect one nerve or many nerves. Some are the result of other diseases, like diabetic nerve problems. Others, like Guillain-Barre syndrome, happen after a virus infection. Still others are from nerve compression, like carpal tunnel syndrome or thoracic outlet syndrome. In some cases, like complex regional pain syndrome and brachial plexus injuries, the problem begins after an injury. However, some people are born with peripheral neurological disorders.

Peripheral nerve stimulation has become established for very specific clinical indications, including certain complex regional pain syndromes, pain due to peripheral nerve injuries, and the like. Some of the common applications of peripheral nerve stimulation include treatment of back pain, occipital nerve stimulation for treatment of migraine headaches, and pudendal nerve stimulation that is being investigated for use in urinary bladder incontinence.

Certain target sites intended to undergo treatment may consist of more than one type of tissue (i.e., nerves, muscles, bone, blood vessels, etc.). In particular, a tissue of interest (i.e., the specific tissue to undergo treatment) may be adjacent to one or more tissues that are not of interest (i.e., tissue that is not intended to undergo treatment). In one scenario, a surgeon may wish to provide electrotherapeutic stimulation to a nerve tissue, while avoiding providing any such stimulation to an adjacent blood vessel, for example, as unintended collateral damage may result in damage to the blood vessel and cause further complications. In such a scenario, the specific type of targeted tissue may generally dictate the level of electrical stimulation required to elicit a desired effect. Furthermore, physical properties of the targeted tissue, including the specific location and depth of the targeted tissue, in relation to the non-targeted tissue, further impacts the level of electrical stimulation necessary to result in effective therapeutic treatment.

The invention solves these problems by providing a treatment device and a console unit for providing intuitive and automated control and targeting of energy output from the treatment device sufficient to ensure successful treatment of a condition, such as a nasal condition, including rhinosinusitis. In particular, the console unit provides a user, via an interactive interface, with comprehensive operational instructions for performing a given procedure and, in response to user input, further provides automatic and precise control over the ablation/modulation of the targeted tissue while minimizing and/or preventing collateral damage to surrounding or adjacent non-targeted tissue at the target site. More specifically, the console unit provides the user with step-by-step guidance, in the form of selectable inputs, for treating, via the treatment device, rhinosinusitis. It should be noted, however, that the systems and methods of the present invention can be used to treat various conditions, and is not limited to the treatment of a nasal condition.

Such step-by-step guidance provided via the interactive interface of the console unit may include, for example, directing the user through the initial set up of the device with the console unit, including authenticating the device (to ensure that the device is in fact suitable and/or authorized to operate with the console unit), and, upon authenticating the device, further directing the user to select a location in which to provide treatment (i.e., left or right nasal cavity). Based on the user's selection of a given nasal cavity, the console unit further provides the user with an indication as to when the device is primed and ready to perform treatment in the selected location. In particular, the console unit is configured to perform an assessment of one or more electrodes associated with an end effector of the treatment device, wherein such assessment includes a determination of whether electrodes are available for use (i.e., via an impedance assessment of each electrode).

Depending on the availability of one or more electrodes for energy delivery, the user may be presented with operational inputs, including the option of initiating treatment. Upon receiving user selection of treatment initiation, the console unit is configured to determine a specific treatment pattern for controlling delivery of energy at a specific level for a specific period of time to the tissue of interest (i.e., the targeted tissue) sufficient to ensure successful ablation/modulation of the targeted tissue while minimizing and/or preventing collateral damage to surrounding or adjacent non-targeted tissue at the target site. More specifically, the console unit has the ability to characterize, prior to a therapeutic treatment, the type of tissue at a target site by sensing at least bioelectric properties of tissue, wherein such characterization includes identifying specific types of tissue present at the target site. For example, different tissue types include different physiological and histological characteristics. As a result of the different characteristics, different tissue types have different associated bioelectrical properties and thus exhibit different behavior in response to application of energy applied thereto. By knowing such properties of a given tissue, the systems and methods are configured to determine a specific treatment pattern for controlling the delivery of energy. In particular, a given treatment pattern may include, for example, a predetermined treatment time, a precise level of energy to be delivered, and a predetermined impedance threshold for that particular tissue.

The console unit is further configured to receive and process real-time feedback data associated with the targeted tissue undergoing treatment and further provide, via the interactive interface, information to the user, specifically related to the ongoing operation of the treatment device as well as a status of the therapy during the procedure, including indications as to whether treatment via respective electrodes is successful (i.e., complete) or unsuccessful (i.e., incomplete). The console unit is further configured to process the feedback data to further ensure that energy delivered is maintained within the scope of the treatment pattern. More specifically, the console unit is configured to automatically control delivery of energy to the targeted tissue based on the processing of the real-time feedback data, wherein such data includes at least impedance measurement data associated with the targeted tissue collected during delivery of energy to the targeted tissue. The controller is configured to process impedance measurement data to detect a slope change event (e.g., an asymptotic rise) within an impedance profile associated with the treatment, wherein, with reference to the predetermined impedance threshold, the slope change event is indicative of whether the ablation/modulation of the targeted tissue is successful. In turn, the controller is configured to automatically control the delivery of energy to the targeted tissue based on real-time monitoring of feedback data, most notably impedance data, to ensure the desired ablation/modulation is achieved. As a result, the console unit is able to ensure that optimal energy is delivered in order to delay the onset of impedance roll-off, until the target ablation/modulation depth is achieved, while maintaining clinically relevant treatment time. Accordingly, the invention solves the problem of causing unnecessary collateral damage to non-targeted tissue during a procedure involving the application of electrotherapeutic stimulation at a target site composed of a variety of tissue types.

Following the delivery of energy from one or more electrodes, resulting in either successful or unsuccessful treatment of respective targeted tissue, the console unit performs post-treatment analysis. The post-treatment analysis includes a determination of any prior treatments performed, including prior use of the electrodes on prior targeted tissue for a given nasal cavity, a status of such prior use, including whether such treatment was successful or unsuccessful, and a determination of any and all further treatments to be performed. In turn, the console unit provides, via the interactive interface, one or more post-procedure inputs from which the user may select for controlling subsequent use of the treatment device to ensure that the overall procedure (i.e., treatment of rhinosinusitis) is completed by ensuring that all portions of targeted tissue undergo treatment.

Accordingly, the systems and methods of the present invention provide an intuitive, user-friendly, and semi-automated means of treating rhinosinusitis conditions, including precise and focused application of energy to the intended targeted tissue without causing collateral and unintended damage or disruption to other tissue and/or structures. Thus, the efficacy of a vidian neurectomy procedure can be achieved with the systems and methods of the present invention without the drawbacks discussed above. Most notably, the console unit provides a user (i.e., surgeon or other medical professional) with relatively simple operational instructions, in the form of step-by-step guidance via an interactive interface, for performing the procedure, such as directing the user to select a specific nasal cavity to treat, providing indications (both visual and audible) as to when the treatment device is ready to perform a given treatment, providing automated control over the delivery of energy to the targeted tissue upon user-selected input to initiate treatment, and further providing a status of therapy during the procedure and after the procedure, including indications (e.g., visual and/or audible) as to whether the treatment is successful or unsuccessful. Accordingly, such treatment is effective at treating rhinosinusitis conditions while greatly reducing the risk of causing lateral damage or disruption to other tissue or structures (i.e., non-targeted tissue, such as blood vessels, bone, and non-targeted neural tissue), thereby reducing the likelihood of unintended complications and side effects.

It should be noted that, although many of the embodiments are described with respect to devices, systems, and methods for therapeutically modulating nerves associated with the peripheral nervous system (PNS) and thus the treatment of peripheral neurological conditions or disorders, other applications and other embodiments in addition to those described herein are within the scope of the present disclosure. For example, at least some embodiments of the present disclosure may be useful for the treatment of other disorders, such as the treatment of disorders associated with the central nervous system.

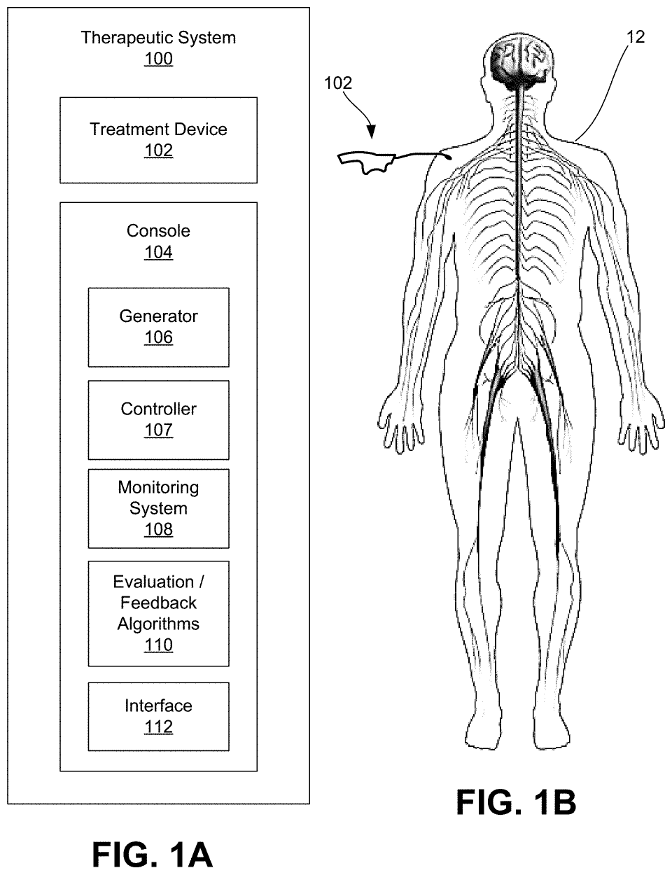

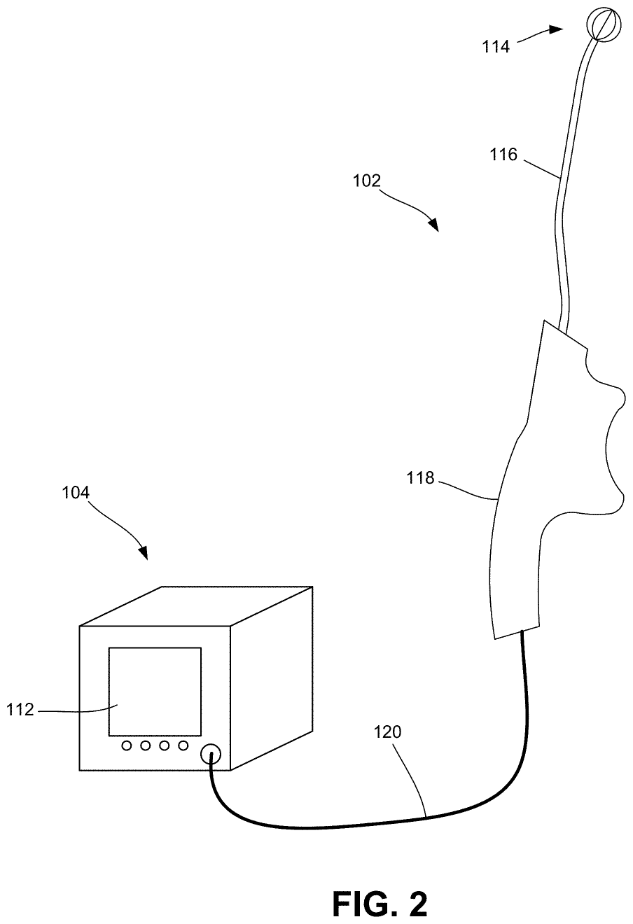

FIGS. 1A and 1B are diagrammatic illustrations of a therapeutic system 100 for treating a condition of a patient using a handheld device 102 according to some embodiments of the present disclosure. The system 100 generally includes a device 102 and a console 104 to which the device 102 is to be connected. FIG. 2 is a diagrammatic illustrations of the console 104 coupled to the handheld device 102 illustrating an exemplary embodiment of an end effector 114 for delivering energy to tissue at the one or more target sites of a patient for the treatment of a neurological disorder. As illustrated, the device 102 is a handheld device, which includes end effector 114, a shaft 116 operably associated with the end effector 114, and a handle 118 operably associated with the shaft 116. The end effector 114 may be collapsible/retractable and expandable, thereby allowing for the end effector 114 to be minimally invasive (i.e., in a collapsed or retracted state) upon delivery to one or more target sites within a patient and then expanded once positioned at the target site. It should be noted that the terms “end effector” and “therapeutic assembly” may be used interchangeably throughout this disclosure.

For example, a surgeon or other medical professional performing a procedure can utilize the handle 118 to manipulate and advance the shaft 116 to a desired target site, wherein the shaft 116 is configured to locate at least a distal portion thereof intraluminally at a treatment or target site within a portion of the patient associated with tissue to undergo electrotherapeutic stimulation for subsequent treatment of an associated condition or disorder. In the event that the tissue to be treated is a nerve, such that electrotherapeutic stimulation thereof results in treatment of an associated neurological condition, the target site may generally be associated with peripheral nerve fibers. The target site may be a region, volume, or area in which the target nerves are located and may differ in size and shape depending upon the anatomy of the patient. Once positioned, the end effector 114 may be deployed and subsequently deliver energy to the one or more target sites. The energy delivered may be non-therapeutic stimulating energy at a frequency for locating neural tissue and further sensing one or more properties of the neural tissue. For example, the end effector 114 may include an electrode array, which includes at least a subset of electrodes configured to sense the presence of neural tissue at a respective position of each of the electrodes, as well as morphology of the neural tissue, wherein such data may be used for determining, via the console 104, the type of neural tissue, depth of neural tissue, and location of neural tissue.

Based on the identification of the neural tissue type, the console 104 is configured to determine a specific treatment pattern for controlling delivery of energy from the end effector 114 upon the target site at a specific level for a specific period of time to the tissue of interest (i.e., the targeted tissue) sufficient to ensure successful ablation/modulation of the targeted tissue while minimizing and/or preventing collateral damage to surrounding or adjacent non-targeted tissue at the target site. Accordingly, the end effector 114 is able to therapeutically modulating nerves of interest, particularly nerves associated with a peripheral neurological conditional or disorder so as to treat such condition or disorder, while minimizing and/or preventing collateral damage.

For example, the end effector 114 may include at least one energy delivery element, such as an electrode, configured to delivery energy to the target tissue which may be used for sensing presence and/or specific properties of tissue (such tissue including, but not limited to, muscle, nerves, blood vessels, bones, etc.) for therapeutically modulating tissues of interest, such as neural tissue. For example, one or more electrodes may be provided by one or more portions of the end effector 114, wherein the electrodes may be configured to apply electromagnetic neuromodulation energy (e.g., radiofrequency (RF) energy) to target sites. In other embodiments, the end effector 114 may include other energy delivery elements configured to provide therapeutic neuromodulation using various other modalities, such as cryotherapeutic cooling, ultrasound energy (e.g., high intensity focused ultrasound (“HIFU”) energy), microwave energy (e.g., via a microwave antenna), direct heating, high and/or low power laser energy, mechanical vibration, and/or optical power.

In some embodiments, the end effector 114 may include one or more sensors (not shown), such as one or more temperature sensors (e.g., thermocouples, thermistors, etc.), impedance sensors, and/or other sensors. The sensors and/or the electrodes may be connected to one or more wires extending through the shaft 116 and configured to transmit signals to and from the sensors and/or convey energy to the electrodes.

As shown, the device 102 is operatively coupled to the console 104 via a wired connection, such as cable 120. It should be noted, however, that the device 102 and console 104 may be operatively coupled to one another via a wireless connection. The console 104 is configured to provide various functions for the device 102, which may include, but is not limited to, controlling, monitoring, supplying, and/or otherwise supporting operation of the device 102. For example, when the device 102 is configured for electrode-based, heat-element-based, and/or transducer-based treatment, the console 104 may include an energy generator 106 configured to generate RF energy (e.g., monopolar, bipolar, or multi-polar RF energy), pulsed electrical energy, microwave energy, optical energy, ultrasound energy (e.g., intraluminally-delivered ultrasound and/or HIFU), direct heat energy, radiation (e.g., infrared, visible, and/or gamma radiation), and/or another suitable type of energy.

In some embodiments, the console 104 may include a controller 107 communicatively coupled to the device 102. However, in the embodiments described herein, the controller 107 may generally be carried by and provided within the handle 118 of the device 102. The controller 107 is configured to initiate, terminate, and/or adjust operation of one or more electrodes provided by the end effector 114 directly and/or via the console 104. For example, the controller 107 can be configured to execute an automated control algorithm and/or to receive control instructions from an operator (e.g., surgeon or other medical professional or clinician). For example, the controller 107 and/or other components of the console 104 (e.g., processors, memory, etc.) can include a computer-readable medium carrying instructions, which when executed by the controller 107, causes the device 102 to perform certain functions (e.g., apply energy in a specific manner, detect impedance, detect temperature, detect nerve locations or anatomical structures, etc.). A memory includes one or more of various hardware devices for volatile and non-volatile storage, and can include both read-only and writable memory. For example, a memory can comprise random access memory (RAM), CPU registers, read-only memory (ROM), and writable non-volatile memory, such as flash memory, hard drives, floppy disks, CDs, DVDs, magnetic storage devices, tape drives, device buffers, and so forth. A memory is not a propagating signal divorced from underlying hardware; a memory is thus non-transitory.

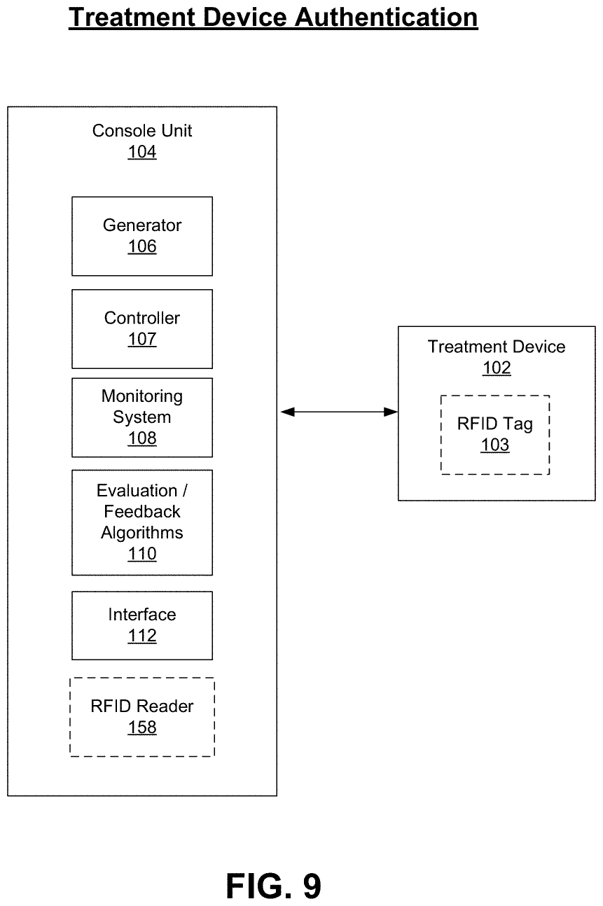

The console 104 may further be configured to provide feedback to an operator before, during, and/or after a treatment procedure via evaluation/feedback algorithms 110. For example, the evaluation/feedback algorithms 110 can be configured to provide information associated with the location of nerves at the treatment site, the temperature of the tissue at the treatment site, and/or the effect of the therapeutic neuromodulation on the nerves at the treatment site. In certain embodiments, the evaluation/feedback algorithm 110 can include features to confirm efficacy of the treatment and/or enhance the desired performance of the system 100. For example, the evaluation/feedback algorithm 110, in conjunction with the controller 107, can be configured to monitor temperature at the treatment site during therapy and automatically shut off the energy delivery when the temperature reaches a predetermined maximum (e.g., when applying RF energy) or predetermined minimum (e.g., when applying cryotherapy). In other embodiments, the evaluation/feedback algorithm 110, in conjunction with the controller 107, can be configured to automatically terminate treatment after a predetermined maximum time, a predetermined maximum impedance rise of the targeted tissue (i.e., in comparison to a baseline impedance measurement), a predetermined maximum impedance of the targeted tissue), and/or other threshold values for biomarkers associated with autonomic function. This and other information associated with the operation of the system 100 can be communicated to the operator via a graphical user interface (GUI) 112 provided via a display on the console 104 and/or a separate display (not shown) communicatively coupled to the console 104, such as a tablet or monitor, to thereby provide visual and/or audible alerts to the operator. The GUI 112 may generally provide operational instructions for the procedure, such as indicating when the device 102 is primed and ready to perform the treatment, and further providing status of therapy during the procedure, including indicating when the treatment is complete, as will be described in greater detail herein, particularly with respect to FIGS. 9 through 14.

For example, as previously described, the end effector 114 and/or other portions of the system 100 can be configured to detect various parameters of a tissue of interest at the target site to determine the anatomy at the target site (e.g., tissue types, tissue locations, vasculature, bone structures, foramen, sinuses, etc.), locate nerves and/or other structures, and allow for neural mapping. For example, the end effector 114 may be configured to detect impedance, dielectric properties, temperature, and/or other properties that indicate the presence of neural tissue or fibers in the target region, as described in greater detail herein.

As shown in FIG. 1A, the console 104 further includes a monitoring system 108 configured to receive data from the end effector 114 (i.e., detected electrical and/or thermal measurements of tissue at the target site), specifically sensed by appropriate sensors (e.g., temperature sensors and/or impedance sensors, or the like), and process this information to identify the presence of nerves, the location of nerves, neural activity at the target site, and/or other properties of the neural tissue, such a physiological properties (e.g., depth), bioelectric properties, and thermal properties. The nerve monitoring system 108 can be operably coupled to the electrodes and/or other features of the end effector 114 via signal wires (e.g., copper wires) that extend through the cable 120 and through the length of the shaft 116. In other embodiments, the end effector 114 can be communicatively coupled to the nerve monitoring system 108 using other suitable communication means.

The nerve monitoring system 108 can determine neural locations and activity before therapeutic neuromodulation to determine precise treatment regions corresponding to the positions of the desired nerves. The nerve monitoring system 108 can further be used during treatment to determine the effect of the therapeutic neuromodulation, and/or after treatment to evaluate whether the therapeutic neuromodulation treated the target nerves to a desired degree. This information can be used to make various determinations related to the nerves proximate to the target site, such as whether the target site is suitable for neuromodulation. In addition, the nerve monitoring system 108 can also compare the detected neural locations and/or activity before and after therapeutic neuromodulation, and compare the change in neural activity to a predetermined threshold to assess whether the application of therapeutic neuromodulation was effective across the treatment site. For example, the nerve monitoring system 108 can further determine electroneurogram (ENG) signals based on recordings of electrical activity of neurons taken by the end effector 114 before and after therapeutic neuromodulation. Statistically meaningful (e.g., measurable or noticeable) decreases in the ENG signal(s) taken after neuromodulation can serve as an indicator that the nerves were sufficiently ablated. Additional features and functions of the nerve monitoring system 108, as well as other functions of the various components of the console 104, including the evaluation/feedback algorithms 110 for providing real-time feedback capabilities for ensuring optimal therapy for a given treatment is administered, are described in at least U.S. Publication No. 2016/0331459 and U.S. Publication No. 2018/0133460, the contents of each of which are incorporated by reference herein in their entireties.

The device 102 provides access to target sites associated with peripheral nerves for the subsequent neuromodulation of such nerves and treatment of a corresponding peripheral neurological condition or disorder. The peripheral nervous system is one of two components that make up the nervous system of bilateral animals, with the other part being the central nervous system (CNS). The PNS consists of the nerves and ganglia outside the brain and spinal cord. The main function of the PNS is to connect the CNS to the limbs and organs, essentially serving as a relay between the brain and spinal cord and the rest of the body. The peripheral nervous system is divided into the somatic nervous system and the autonomic nervous system. In the somatic nervous system, the cranial nerves are part of the PNS with the exception of the optic nerve (cranial nerve II), along with the retina. The second cranial nerve is not a true peripheral nerve but a tract of the diencephalon. Cranial nerve ganglia originated in the CNS. However, the remaining ten cranial nerve axons extend beyond the brain and are therefore considered part of the PNS. The autonomic nervous system exerts involuntary control over smooth muscle and glands. The connection between CNS and organs allows the system to be in two different functional states: sympathetic and parasympathetic. Accordingly, the devices, systems, and methods of the present invention are useful in detecting, identifying, and precision targeting nerves associated with the peripheral nervous system for treatment of corresponding peripheral neurological conditions or disorders.

The peripheral neurological conditions or disorders may include, but are not limited to, chronic pain, movement disorders, epilepsy, psychiatric disorders, cardiovascular disorders, gastrointestinal disorders, genitourinary disorders, to name a few. For example, chronic pain may include headaches, complex regional pain syndrome, neuropathy, peripheral neuralgia, ischemic pain, failed back surgery syndrome, and trigeminal neuralgia. The movement disorders may include spasticity, Parkinson's disease, tremor, dystonia, Tourette syndrome, camptocormia, hemifacial spasm, and Meige syndrome. The psychiatric disorders may include depression, obsessive compulsive disorder, drug addiction, and anorexia/eating disorders. The functional restoration may include restoration of certain functions post traumatic brain injury, hearing impairment, and blindness. The cardiovascular disorders may include angina, heart failure, hypertension, peripheral vascular disorders, and stroke. The gastrointestinal disorders may include dysmotility and obesity. The genitourinary disorders may include painful bladder syndrome, interstitial cystitis, and voiding dysfunction.

For example, the system 100 may be used for the treatment of a cardiovascular disorder, such as arrhythmias or heart rhythm disorders, including, but not limited to, atrial fibrillation (AF or A-fib). Atrial fibrillation is an irregular and often rapid heart rate that can increase one's risk of stroke, heart failure, and other heart-related complications. Atrial fibrillation occurs when regions of cardiac tissue abnormally conduct electric signals to adjacent tissue, thereby disrupting the normal cardiac cycle and causing asynchronous rhythm. Atrial fibrillation symptoms often include heart palpitations, shortness of breath, and weakness. While episodes of atrial fibrillation can come and go, a person may develop atrial fibrillation that doesn't go away and thus will require treatment. Although atrial fibrillation itself usually isn't life-threatening, it is a serious medical condition that sometimes requires emergency treatment, as it may lead to complications. For example, atrial fibrillation is associated with an increased risk of heart failure, dementia, and stroke.

The normal electrical conduction system of the heart allows the impulse that is generated by the sinoatrial node (SA node) of the heart to be propagated to and stimulate the myocardium (muscular layer of the heart). When the myocardium is stimulated, it contracts. It is the ordered stimulation of the myocardium that allows efficient contraction of the heart, thereby allowing blood to be pumped to the body. In AF, the normal regular electrical impulses generated by the sinoatrial node in the right atrium of the heart are overwhelmed by disorganized electrical impulses usually originating in the roots of the pulmonary veins. This leads to irregular conduction of ventricular impulses that generate the heartbeat. In particular, during AF, the heart's two upper chambers (the atria) beat chaotically and irregularly, out of coordination with the two lower chambers (the ventricles) of the heart.

During atrial fibrillation, the regular impulses produced by the sinus node for a normal heartbeat are overwhelmed by rapid electrical discharges produced in the atria and adjacent parts of the pulmonary veins. Sources of these disturbances are either automatic foci, often localized at one of the pulmonary veins, or a small number of localized sources in the form of either a re-entrant leading circle, or electrical spiral waves (rotors). These localized sources may be found in the left atrium near the pulmonary veins or in a variety of other locations through both the left or right atrium. There are three fundamental components that favor the establishment of a leading circle or a rotor: 1) slow conduction velocity of cardiac action potential; 2) short refractory period; and 3) small wavelength. Wavelength is the product of velocity and refractory period. If the action potential has fast conduction, with a long refractory period and/or conduction pathway shorter than the wavelength, an AF focus would not be established. In multiple wavelet theory, a wavefront will break into smaller daughter wavelets when encountering an obstacle, through a process called vortex shedding; but under proper conditions, such wavelets can reform and spin around a center, forming an AF focus.

The system 100 provides for the treatment of AF, in which the device 102 may provide access to and provide treatment of one or more target sites associated with nerves that correspond to, or are otherwise associated with, treating AF. For example, the device 102, in conjunction with the console 104, may detect, identify, and precision target cardiac tissue and subsequently deliver energy at a level or frequency sufficient to therapeutically modulate nerves associated with such cardiac tissue. The therapeutic modulation of such nerves is sufficient to disrupt the origin of the signals causing the AF and/or disrupt the conducting pathway for such signals.

Similar to the conduction system of the heart, a neural network exists which surrounds the heart and plays an important role in formation of the substrate of AF and when a trigger is originated, usually from pulmonary vein sleeves, AF occurs. This neural network includes ganglionated plexi (GP) located adjacent to pulmonary vein ostia which are under control of higher centers in normal people. For example, the heart is richly innervated by the autonomic nerves. The ganglion cells of the autonomic nerves are located either outside the heart (extrinsic) or inside the heart (intrinsic). Both extrinsic and intrinsic nervous systems are important for cardiac function and arrhythmogenesis. The vagal nerves include axons that come from various nuclei in the medulla. The extrinsic sympathetic nerves come from the paravertebral ganglia, including the superior cervical ganglion, middle cervical ganglion, the cervicothoracic (stellate) ganglion and the thoracic ganglia. The intrinsic cardiac nerves are found mostly in the atria, and are intimately involved in atrial arrhythmogenesis cardiovascular disorder, such as arrhythmias or heart rhythm disorders, including, but not limited to, atrial fibrillation. When GP become hyperactive owing to loss of inhibition from higher centers (e.g., in elderly), AF can occur.

The system 100 can be used to control hyperactive GP either by stimulating higher centers and their connections, such as vagus nerve stimulation, or simply by ablating GP. Accordingly, the device 102, in conjunction with the console 104, may detect and identify ganglionated plexus (GP) and further determine an energy level sufficient to therapeutically modulate or treat (i.e., ablate) the GP for the treatment of AF (i.e., surgically disrupting the origin of the signals causing the AF and disrupting the conducting pathway for such signals) while minimizing and/or preventing collateral damage to surrounding or adjacent non-neural tissue including bloods vessels and bone and non-targeted neural tissue. It should be noted that other nerves and/or cardiac tissue, or other structures, known to have an impact on or cause AF, may be targeted by the system 100, including, but not limited to, pulmonary veins (e.g., pulmonary vein isolation upon creation of lesions around PV ostia to prevent triggers from reaching atrial substrate).

In addition to treating arrhythmias, the system 100 may also be used for the treatment of other cardiovascular-related conditions, particularly those involving the kidney. The kidneys play a significant role in the progression of CHF, as well as in Chronic Renal Failure (CRF), End-Stage Renal Disease (ESRD), hypertension (pathologically high blood pressure), and other cardio-renal diseases.

The functions of the kidney can be summarized under three broad categories: filtering blood and excreting waste products generated by the body's metabolism; regulating salt, water, electrolyte and acid-base balance; and secreting hormones to maintain vital organ blood flow. Without properly functioning kidneys, a patient will suffer water retention, reduced urine flow and an accumulation of waste toxins in the blood and body. These conditions resulting from reduced renal function or renal failure (kidney failure) are believed to increase the workload of the heart.

For example, in a CHF patient, renal failure will cause the heart to further deteriorate as the water build-up and blood toxins accumulate due to the poorly functioning kidneys and, in turn, cause the heart further harm. CHF is a condition that occurs when the heart becomes damaged and reduces blood flow to the organs of the body. If blood flow decreases sufficiently, kidney function becomes impaired and results in fluid retention, abnormal hormone secretions and increased constriction of blood vessels. These results increase the workload of the heart and further decrease the capacity of the heart to pump blood through the kidney and circulatory system. This reduced capacity further reduces blood flow to the kidney. It is believed that progressively decreasing perfusion of the kidney is a principal non-cardiac cause perpetuating the downward spiral of CHF. Moreover, the fluid overload and associated clinical symptoms resulting from these physiologic changes are predominant causes for excessive hospital admissions, reduced quality of life, and overwhelming costs to the health care system due to CHF.

End-stage renal disease is another condition at least partially controlled by renal neural activity. There has been a dramatic increase in patients with ESRD due to diabetic nephropathy, chronic glomerulonephritis and uncontrolled hypertension. Chronic renal failure (CRF) slowly progresses to ESRD. CRF represents a critical period in the evolution of ESRD. The signs and symptoms of CRF are initially minor, but over the course of 2-5 years, become progressive and irreversible. While some progress has been made in combating the progression to, and complications of, ESRD, the clinical benefits of existing interventions remain limited.

Arterial hypertension is a major health problem worldwide. Treatment-resistant hypertension is defined as the failure to achieve target blood pressure despite the concomitant use of maximally tolerated doses of three different antihypertensive medications, including a diuretic. Treatment-resistant hypertension is associated with considerable morbidity and mortality. Patients with treatment-resistant hypertension have markedly increased cardiovascular morbidity and mortality, facing an increase in the risk of myocardial infarction (MI), stroke, and death compared to patients whose hypertension is adequately controlled.

The autonomic nervous system is recognized as an important pathway for control signals that are responsible for the regulation of body functions critical for maintaining vascular fluid balance and blood pressure. The autonomic nervous system conducts information in the form of signals from the body's biologic sensors such as baroreceptors (responding to pressure and volume of blood) and chemoreceptors (responding to chemical composition of blood) to the central nervous system via its sensory fibers. It also conducts command signals from the central nervous system that control the various innervated components of the vascular system via its motor fibers.

It is known from clinical experience and research that an increase in renal sympathetic nerve activity leads to vasoconstriction of blood vessels supplying the kidney, decreased renal blood flow, decreased removal of water and sodium from the body, and increased renin secretion. It is also known that reduction of sympathetic renal nerve activity, e.g., via denervation, may reverse these processes.

The renal sympathetic nervous system plays a critical influence in the pathophysiology of hypertension. The adventitia of the renal arteries has efferent and afferent sympathetic nerves. Renal sympathetic activation via the efferent nerves initiates a cascade resulting in elevated blood pressure. Efferent sympathetic outflow leads to vasoconstriction with a subsequent reduction in glomerular blood flow, a lowering of the glomerular filtration rate, release of renin by the juxtaglomerular cells, and the subsequent activation of the renin-angiotensin-aldosterone axis leading to increased tubular reabsorption of sodium and water. Decreased glomerular filtration rate also prompts additional systemic sympathetic release of catecholamines. As a consequence, blood pressure increases by a rise in total blood volume and increased peripheral vascular resistance.

The system 100 can be used for the treatment of cardio-renal diseases, including hypertension, by providing renal neuromodulation and/or denervation. For example, the device 102 may be placed at one or more target sites associated with renal nerves other neural fibers that contribute to renal neural function, or other neural features. For example, the device 102, in conjunction with the console 104, may detect, identify, and precision target renal nerve tissue and subsequently deliver energy at a level or frequency sufficient to therapeutically modulate nerves associated with such renal tissue. The therapeutic modulation of such renal nerves and/or renal tissue is sufficient to completely block or denervate the target neural structures and/or disrupt renal nervous activity, while minimizing and/or preventing collateral damage to surrounding or adjacent non-neural tissue including bloods vessels and bone and non-targeted neural tissue.

It should further be noted that the system 100 can be used to determine disease progression. In particular, the present system 100 can obtain measurements at one or more target sites associated with a given disease, disorder, or the like. Such measurements may be based on the active neural parameters (i.e., neuronal firing and active voltage monitoring) and may be used to identify neurons. The active neural parameters (and thus behavior) change with disease progression, thereby allowing the present system to identify such changes and determine a progression of the underlying disease or disorder. Such capabilities are possible based, at least in part, on the fact that the present system 100 is configured to monitor passive electric phenomena (i.e., the present system 100 determines the ohmic conductivity frequency, which remains consistent, while conductivity will be different based on disease or disorder progression).

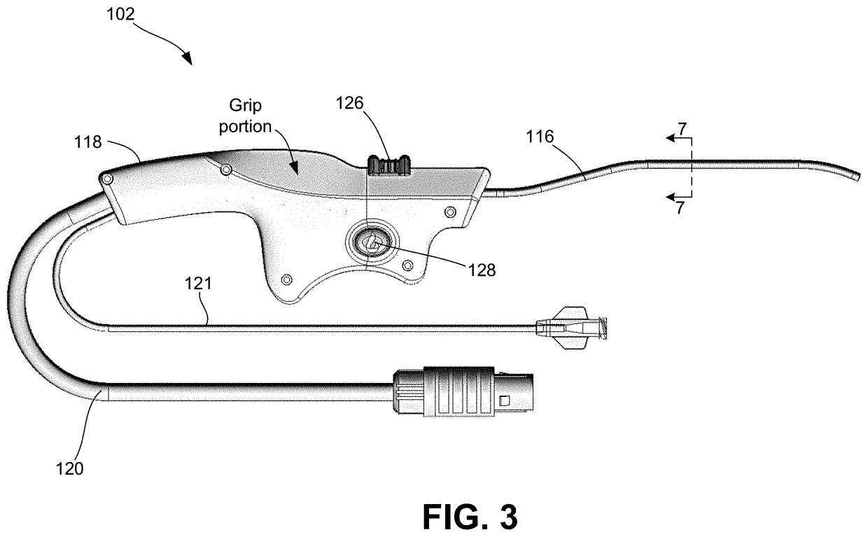

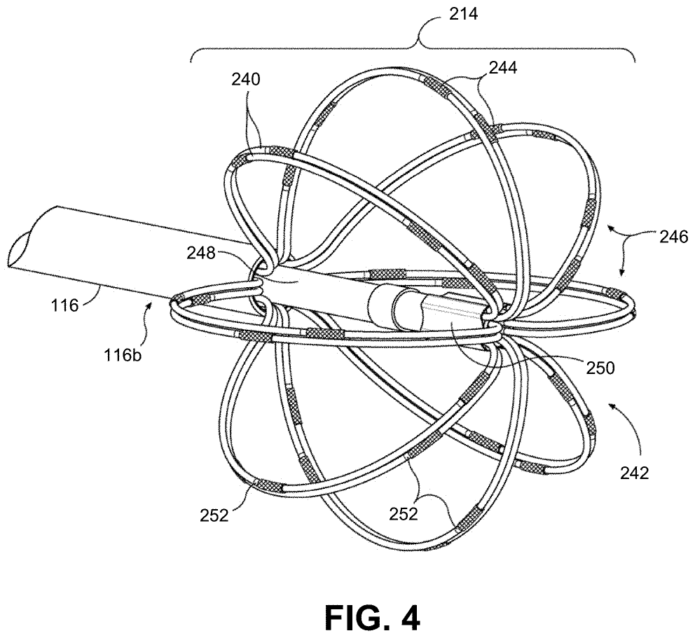

FIG. 3 is a side view of one embodiment of a handheld device for providing therapeutic neuromodulation consistent with the present disclosure. As previously described, the device 102 includes an end effector (not shown) transformable between a collapsed/retracted configuration and an expanded deployed configuration, a shaft 116 operably associated with the end effector, and a handle 118 operably associated with the shaft 116. The handle 118 includes at least a first mechanism 126 for deployment of the end effector from collapsed/retracted configuration to the expanded, deployed configuration, and a second mechanism 128, separate from the first mechanism 124, for control of energy output by the end effector, specifically electrodes or other energy elements provided by the end effector. The handheld device 102 may further include an auxiliary line 121, which may provide a fluid connection between a fluid source, for example, and the shaft 116 such that fluid may be provided to a target site via the distal end of the shaft 116. In some embodiments, the auxiliary line 121 may provide a connection between a vacuum source and the shaft 116, such that the device 102 may include suction capabilities (via the distal end of the shaft 116).

FIG. 4 is an enlarged, perspective view of one embodiment of an end effector 214 consistent with the present disclosure. As shown, the end effector 214 is generally positioned at a distal portion 116b of the shaft 116. The end effector 214 is transformable between a low-profile delivery state to facilitate intraluminal delivery of the end effector 214 to a treatment site and an expanded state, as shown. The end effector 214 includes a plurality of struts 240 that are spaced apart from each other to form a frame or basket 242 when the end effector 214 is in the expanded state. The struts 240 can carry one or more energy delivery elements, such as a plurality of electrodes 244. In the expanded state, the struts 240 can position at least two of the electrodes 244 against tissue at a target site within a particular region. The electrodes 244 can apply bipolar or multi-polar RF energy to the target site to therapeutically modulate nerves associated with a peripheral neurological condition or disorder. In various embodiments, the electrodes 244 can be configured to apply pulsed RF energy with a desired duty cycle (e.g., 1 second on/0.5 seconds off) to regulate the temperature increase in the target tissue.

In the embodiment illustrated in FIG. 4, the basket 242 includes eight branches 246 spaced radially apart from each other to form at least a generally spherical structure, and each of the branches 246 includes two struts 240 positioned adjacent to each other. In other embodiments, however, the basket 242 can include fewer than eight branches 246 (e.g., two, three, four, five, six, or seven branches) or more than eight branches 246. In further embodiments, each branch 246 of the basket 242 can include a single strut 240, more than two struts 240, and/or the number of struts 240 per branch can vary. In still further embodiments, the branches 246 and struts 240 can form baskets or frames having other suitable shapes for placing the electrodes 244 in contact with tissue at the target site. For example, when in the expanded state, the struts 240 can form an ovoid shape, a hemispherical shape, a cylindrical structure, a pyramid structure, and/or other suitable shapes.

The end effector 214 can further include an internal or interior support member 248 that extends distally from the distal portion 116b of the shaft 116. A distal end portion 250 of the support member 248 can support the distal end portions of the struts 240 to form the desired basket shape. For example, the struts 240 can extend distally from the distal portion 116b of the shaft 116 and the distal end portions of the struts 240 can attach to the distal end portion 250 of the support member 248. In certain embodiments, the support member 248 can include an internal channel (not shown) through which electrical connectors (e.g., wires) coupled to the electrodes 244 and/or other electrical features of the end effector 214 can run. In various embodiments, the internal support member 248 can also carry an electrode (not shown) at the distal end portion 250 and/or along the length of the support member 248.

The basket 242 can transform from the low-profile delivery state to the expanded state (shown in FIG. 4) by either manually manipulating a handle of the device 102, interacting with the first mechanism 126 for deployment of the end effector 214 from collapsed/retracted configuration to the expanded, deployed configuration, and/or other feature at the proximal portion of the shaft 116 and operably coupled to the basket 242. For example, to move the basket 242 from the expanded state to the delivery state, an operator can push the support member 248 distally to bring the struts 240 inward toward the support member 248. An introducer or guide sheath (not shown) can be positioned over the low-profile end effector 214 to facilitate intraluminal delivery or removal of the end effector 214 from or to the target site. In other embodiments, the end effector 214 is transformed between the delivery state and the expanded state using other suitable means, such as the first mechanism 126, as will be described in greater detail herein.

The individual struts 240 can be made from a resilient material, such as a shape-memory material (e.g., Nitinol) that allows the struts 240 to self-expand into the desired shape of the basket 242 when in the expanded state. In other embodiments, the struts 240 can be made from other suitable materials and/or the end effector 214 can be mechanically expanded via a balloon or by proximal movement of the support member 248. The basket 242 and the associated struts 240 can have sufficient rigidity to support the electrodes 244 and position or press the electrodes 244 against tissue at the target site. In addition, the expanded basket 242 can press against surrounding anatomical structures proximate to the target site and the individual struts 240 can at least partially conform to the shape of the adjacent anatomical structures to anchor the end effector 214 at the treatment site during energy delivery. In addition, the expansion and conformability of the struts 240 can facilitate placing the electrodes 244 in contact with the surrounding tissue at the target site.

At least one electrode 244 is disposed on individual struts 240. In the illustrated embodiment, two electrodes 244 are positioned along the length of each strut 240. In other embodiments, the number of electrodes 244 on individual struts 240 be only one, more than two, zero, and/or the number of electrodes 244 on the different struts 240 can vary. The electrodes 244 can be made from platinum, iridium, gold, silver, stainless steel, platinum-iridium, cobalt chromium, iridium oxide, polyethylenedioxythiophene (“PEDOT”), titanium, titanium nitride, carbon, carbon nanotubes, platinum grey, Drawn Filled Tubing (“DFT”) with a silver core made by Fort Wayne Metals of Fort Wayne, Ind., and/or other suitable materials for delivery RF energy to target tissue.

In certain embodiments, each electrode 444 can be operated independently of the other electrodes 244. For example, each electrode can be individually activated and the waveform, polarity and amplitude of each electrode can be selected by an operator or a control algorithm (e.g., executed by the controller 107 of FIG. 1A). Various embodiments of such independently controlled electrodes 244 are described in greater detail herein. The selective independent control of the electrodes 244 allows the end effector 214 to deliver RF energy to highly customized regions and to further create multiple micro-lesions to selectively modulate a target neural structure by effectively causing multi-point interruption of a neural signal due to the multiple micro-lesions. For example, a select portion of the electrodes 244 can be activated to target neural fibers in a specific region while the other electrodes 244 remain inactive. In certain embodiments, for example, electrodes 244 may be activated across the portion of the basket 242 that is adjacent to tissue at the target site, and the electrodes 244 that are not proximate to the target tissue can remain inactive to avoid applying energy to non-target tissue. Such configurations facilitate selective therapeutic modulation of nerves along a portion of a target site without applying energy to structures in other portions of the target site.

The electrodes 244 can be electrically coupled to an RF generator (e.g., the generator 106 of FIG. 1A) via wires (not shown) that extend from the electrodes 244, through the shaft 116, and to the RF generator. When each of the electrodes 244 is independently controlled, each electrode 244 couples to a corresponding wire that extends through the shaft 116. In other embodiments, multiple electrodes 244 can be controlled together and, therefore, multiple electrodes 244 can be electrically coupled to the same wire extending through the shaft 116. The RF generator and/or components operably coupled (e.g., a control module) thereto can include custom algorithms to control the activation of the electrodes 244. For example, the RF generator can deliver RF power at about 200-300 W to the electrodes 244, and do so while activating the electrodes 244 in a predetermined pattern selected based on the position of the end effector 214 relative to the treatment site and/or the identified locations of the target nerves. In other embodiments, the RF generator delivers power at lower levels (e.g., less than 1 W, 2-5W, 5-15 W, 15-50 W, 50-150 W, etc.) and/or higher power levels.

The end effector 214 can further include one or more sensors 252 (e.g., temperature sensors, impedance sensors, etc.) disposed on the struts 240 and/or other portions of the end effector 214 and configured to sense/detect one or more properties associated with tissue at a target site. For example, temperature sensors are configured to detect the temperature adjacent thereto. The sensors 252 can be electrically coupled to a console (e.g., the console 104 of FIG. 1A) via wires (not shown) that extend through the shaft 116. In various embodiments, the sensors 252 can be positioned proximate to the electrodes 244 to detect various properties of targeted tissue and/or the treatment associated therewith. As will be described in greater detail herein, the sensed data can be provided to the console 104, wherein such data is generally related to at least bioelectric properties of tissue at the target site. In turn, the console 104 (via the controller 107, monitoring system 108, and evaluation/feedback algorithms 110) is configured to process such data and determine to identify a type of each of the one or more tissues at the target site. The console (via the controller 107, monitoring system 108, and evaluation/feedback algorithms 110) is further configured to determine a treatment pattern (also referred to herein as “ablation pattern”) to be delivered by one or more of the plurality of electrodes of the end effector based on the tissue type, as well as tissue location and/or depth. The ablation energy associated with the ablation pattern is at a level sufficient to ablate a targeted tissue and minimize and/or prevent collateral damage to surrounding or adjacent non-targeted tissue at the target site. In particular, a given treatment pattern may include, for example, a predetermined treatment time, a precise level of energy to be delivered, and a predetermined impedance threshold for that particular tissue.

The device 102 is further be configured to provide the console 104 with sensed data in the form of feedback data, in real-, or near-real, time. The real-time feedback data is associated with the effect of the therapeutic stimulation on the targeted tissue. For example, feedback data may be associated with efficacy of ablation upon targeted tissue (e.g., neural tissue) during and/or after delivery of initial energy from one or more of the plurality of electrodes. Accordingly, the console 104 (via the controller 107, monitoring system 108, and evaluation/feedback algorithms 110) is configured to process such real-time feedback data to determine if certain properties of the targeted tissue undergoing treatment (e.g., tissue temperature, tissue impedance, etc.) reach predetermined thresholds for irreversible tissue damage.

More specifically, the console 104 (via the controller 107, monitoring system 108, and evaluation/feedback algorithms 110) is configured to automatically control delivery of energy to the targeted tissue based on the processing of the real-time feedback data, wherein such data includes at least impedance measurement data associated with the targeted tissue collected during delivery of energy to the targeted tissue. The console 104 (via the controller 107, monitoring system 108, and evaluation/feedback algorithms 110) is configured to process impedance measurement data to detect a slope change event (e.g., an asymptotic rise) within an impedance profile associated with the treatment, wherein, with reference to the predetermined impedance threshold, the slope change event is indicative of whether the ablation/modulation of the targeted tissue is successful. In turn, the controller 107 can automatically tune energy output individually for the one or more electrodes after an initial level of energy has been delivered based, at least in part, on monitoring and processing of the real-time feedback data, most notably impedance data, to ensure the desired ablation/modulation is achieved. For example, once a slope change event (e.g., an asymptotic rise) within an impedance profile is detected, with reference to the predetermined impedance threshold of the targeted tissue (which is known via the treatment pattern), the application of therapeutic neuromodulation energy can be terminated to allow the tissue to remain intact and to further prevent and/or minimize collateral damage to surrounding or adjacent non-targeted tissue. For example, in certain embodiments, the energy delivery can automatically be tuned based on an evaluation/feedback algorithm (e.g., the evaluation/feedback algorithm 110 of FIG. 1A) stored on a console (e.g., the console 104 of FIG. 1A) operably coupled to the end effector 214.

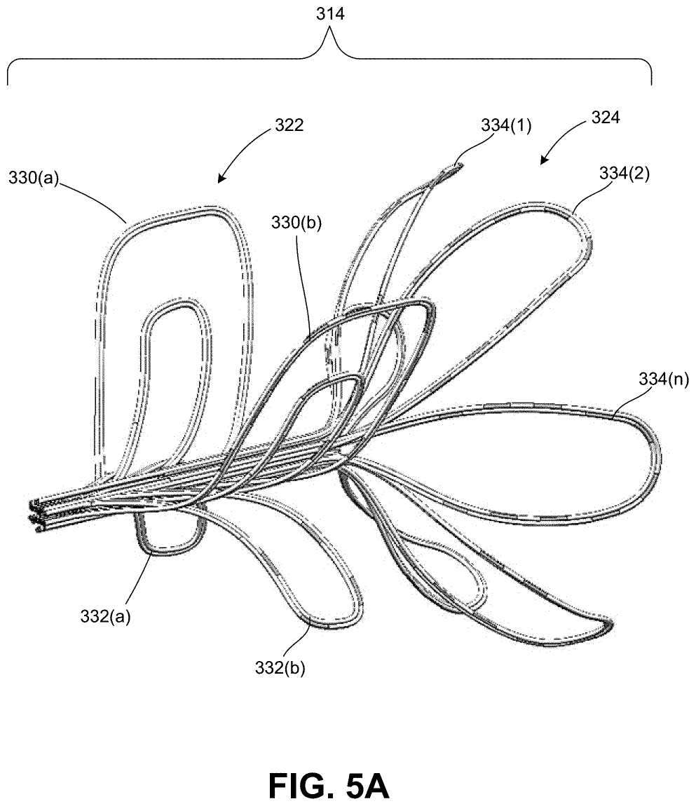

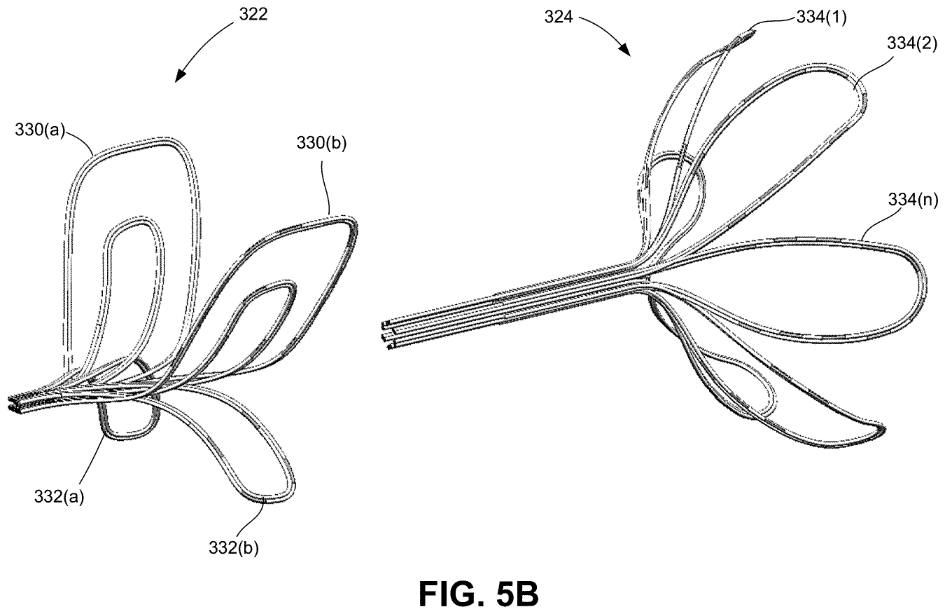

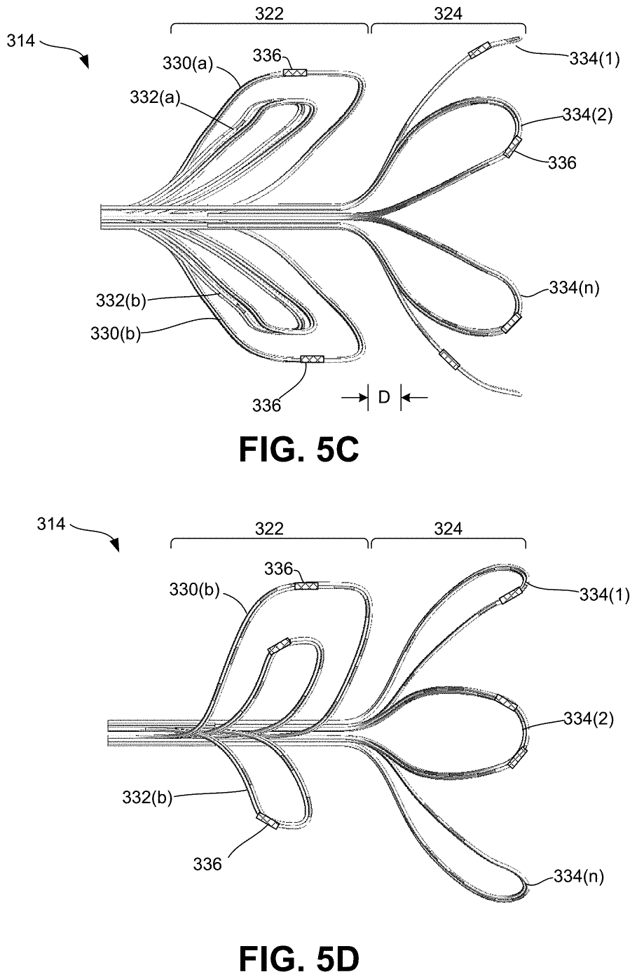

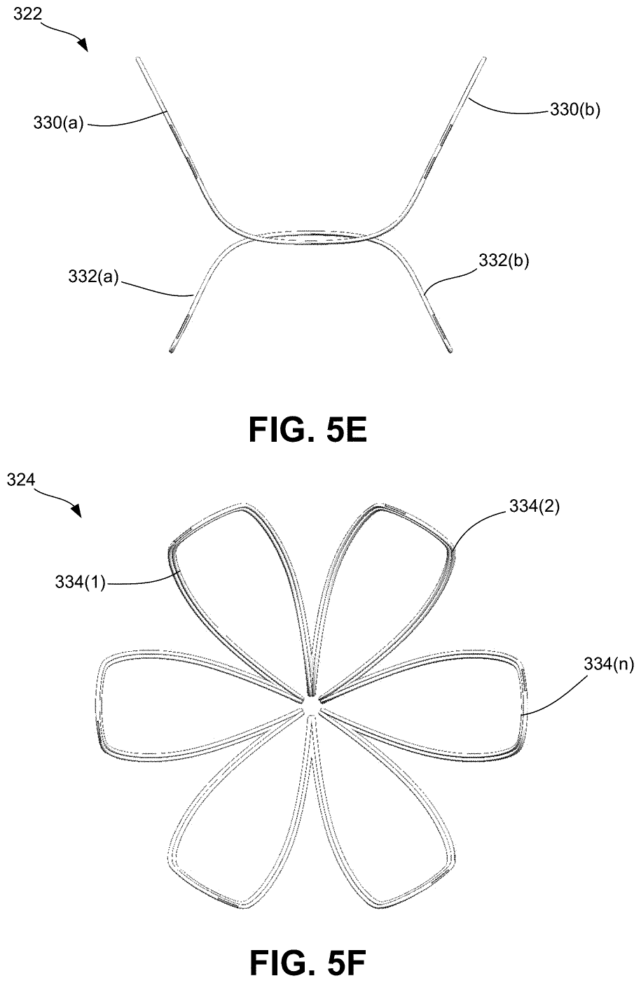

FIGS. 5A-5F are various views of another embodiment of an end effector 314 consistent with the present disclosure. As generally illustrated, the end effector 314 is a multi-segmented end effector, which includes at least a first segment 322 and a second segment 324 spaced apart from one another. The first segment 322 is generally positioned closer to a distal portion of the shaft 116, and is thus sometimes referred to herein as the proximal segment 322, while the second segment 324 is generally positioned further from the distal portion of the shaft 116 and is thus sometimes referred to herein as the distal segment 324. Each of the first and second segments 322 and 324 is transformable between a retracted configuration, which includes a low-profile delivery state and a deployed configuration, which includes an expanded state, as shown in the figures. The end effector 314 is generally designed to be positioned within a nasal region of the patient for the treatment of a rhinosinusitis condition while minimizing or avoiding collateral damage to surrounding tissue, such as blood vessels or bone. In particular, the end effector 314 is configured to be advanced within the nasal cavity and be positioned at one or more target sites generally associated with postganglionic parasympathetic fibers that innervate the nasal mucosa. In turn, the end effector 314 is configured to therapeutically modulate the postganglionic parasympathetic nerves.

It should be noted, however, that an end effector consistent with the present disclosure may be multi-segmented in a similar fashion as end effector 314 and may be used to provide treatment in other regions of the patient outside of the nasal cavity and thus is not limited to the particular design/configuration as the end effector 314 nor the intended treatment site (e.g., nasal cavity). Rather, other multi-segmented designs are contemplated for use in particular regions of a patient, particularly regions in which the use of multiple and distinct segments would be advantageous, as is the case with the end effector 314 design due to the anatomy of the nasal cavity.

FIG. 5A is an enlarged, perspective view of the multi-segment end effector illustrating the first (proximal) segment 322 and second (distal) segment 324. FIG. 5B is an exploded, perspective view of the multi-segment end effector 314. FIG. 5C is an enlarged, top view of the multi-segment end effector 314. FIG. 5D is an enlarged, side view of the multi-segment end effector 314. FIG. 5E is an enlarged, front (proximal facing) view of the first (proximal) segment 322 of the multi-segment end effector 314 and FIG. 5F is an enlarged, front (proximal facing) view of the second (distal) segment 324 of the multi-segment end effector 314.

As illustrated, the first segment 322 includes at least a first set of flexible support elements, generally in the form of wires, arranged in a first configuration, and the second segment 324 includes a second set of flexible support elements, also in the form of wires, arranged in a second configuration. The first and second sets of flexible support elements include composite wires having conductive and elastic properties. For example, in some embodiments, the composite wires include a shape memory material, such as Nitinol. The flexible support elements may further include a highly lubricious coating, which may allow for desirable electrical insulation properties as well as desirable low friction surface finish. Each of the first and second segments 322, 324 is transformable between a retracted configuration and an expanded deployed configuration such that the first and second sets of flexible support elements are configured to position one or more electrodes provided on the respective segments (see electrodes 336 in FIGS. 5E and 5F) into contact with one or more target sites when in the deployed configuration.

As shown, when in the expanded deployed configuration, the first set of support elements of the first segment 322 includes at least a first pair of struts 330a,330b, each comprising a loop (or leaflet) shape and extending in an upward direction and a second pair of struts 332a, 332b, each comprising a loop (or leaflet) shape and extending in a downward direction, generally in an opposite direction relative to at least the first pair of struts 330a, 330b. It should be noted that the terms upward and downward are used to describe the orientation of the first and second segments 322, 324 relative to one another. More specifically, the first pair of struts 330a, 330b generally extend in an outward inclination in a first direction relative to a longitudinal axis of the multi-segment end effector 314 and are spaced apart from one another. Similarly, the second pair of struts 332a, 332b extend in an outward inclination in a second direction substantially opposite the first direction relative to the longitudinal axis of the multi-segment end effector and spaced apart from one another.

The second set of support elements of the second segment 324, when in the expanded deployed configuration, includes a second set of struts 334(1), 334(2), 334(n) (approximately six struts), each comprising a loop shape extending outward to form an open-ended circumferential shape. As shown, the open-ended circumferential shape generally resembles a blooming flower, wherein each looped strut 334 may generally resemble a flower petal. It should be noted that the second set of struts 334 may include any number of individual struts and is not limited to six, as illustrated. For example, in some embodiments, the second segment 124 may include two, three, four, five, six, seven, eight, nine, ten, or more struts 334.

The first and second segments 322, 324, specifically struts 330, 332, and 334 include one or more energy delivery elements, such as a plurality of electrodes 336. It should be noted that any individual strut may include any number of electrodes 336 and is not limited to one electrode, as shown. In the expanded state, the struts 330, 332, and 334 can position any number of electrodes 336 against tissue at a target site within the nasal region (e.g., proximate to the palatine bone inferior to the SPF). The electrodes 336 can apply bipolar or multi-polar radiofrequency (RF) energy to the target site to therapeutically modulate postganglionic parasympathetic nerves that innervate the nasal mucosa proximate to the target site. In various embodiments, the electrodes 336 can be configured to apply pulsed RF energy with a desired duty cycle (e.g., 1 second on/0.5 seconds off) to regulate the temperature increase in the target tissue.

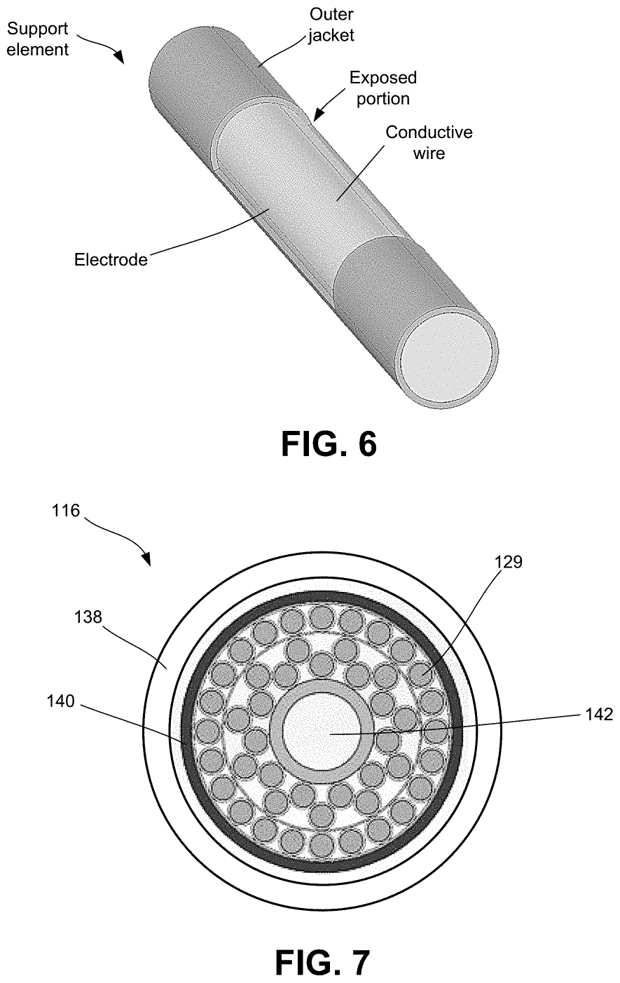

The first and second segments 322, 324 and the associated struts 330, 332, and 334 can have sufficient rigidity to support the electrodes 336 and position or press the electrodes 336 against tissue at the target site. In addition, each of the expanded first and second segments 322, 324 can press against surrounding anatomical structures proximate to the target site (e.g., the turbinates, the palatine bone, etc.) and the individual struts 330, 332, 334 can at least partially conform to the shape of the adjacent anatomical structures to anchor the end effector 314. In addition, the expansion and conformability of the struts 330, 332, 334 can facilitate placing the electrodes 336 in contact with the surrounding tissue at the target site. The electrodes 336 can be made from platinum, iridium, gold, silver, stainless steel, platinum-iridium, cobalt chromium, iridium oxide, polyethylenedioxythiophene (PEDOT), titanium, titanium nitride, carbon, carbon nanotubes, platinum grey, Drawn Filled Tubing (DFT) with a silver core, and/or other suitable materials for delivery RF energy to target tissue. In some embodiments, such as illustrated in FIG. 6, a strut may include an outer jacket surrounding a conductive wire, wherein portions of the outer jacket are selectively absent along a length of the strut, thereby exposing the underlying conductive wire so as to act as an energy delivering element (i.e., an electrode) and/or sensing element, as described in greater detail herein.

In certain embodiments, each electrode 336 can be operated independently of the other electrodes 336. For example, each electrode can be individually activated and the polarity and amplitude of each electrode can be selected by an operator or a control algorithm (e.g., executed by the controller 107 previously described herein). The selective independent control of the electrodes 336 allows the end effector 314 to deliver RF energy to highly customized regions. For example, a select portion of the electrodes 336 can be activated to target neural fibers in a specific region while the other electrodes 336 remain inactive. In certain embodiments, for example, electrodes 136 may be activated across the portion of the second segment 324 that is adjacent to tissue at the target site, and the electrodes 336 that are not proximate to the target tissue can remain inactive to avoid applying energy to non-target tissue. Such configurations facilitate selective therapeutic modulation of nerves on the lateral nasal wall within one nostril without applying energy to structures in other portions of the nasal cavity.

The electrodes 336 are electrically coupled to an RF generator (e.g., the generator 106 of FIG. 1A) via wires (not shown) that extend from the electrodes 336, through the shaft 116, and to the RF generator. When each of the electrodes 336 is independently controlled, each electrode 336 couples to a corresponding wire that extends through the shaft 116. In other embodiments, multiple electrodes 336 can be controlled together and, therefore, multiple electrodes 336 can be electrically coupled to the same wire extending through the shaft 116. As previously described, the RF generator and/or components operably coupled (e.g., a control module) thereto can include custom algorithms to control the activation of the electrodes 336. For example, the RF generator can deliver RF power at about 460-480 kHz (+ or −5 kHz) to the electrodes 336, and do so while activating the electrodes 336 in a predetermined pattern selected based on the position of the end effector 314 relative to the treatment site and/or the identified locations of the target tissues. It should further be noted that the electrodes 336 may be individually activated and controlled (i.e., controlled level of energy output and delivery) based, at least in part, on feedback data. The RF generator is able to provide bipolar low power (10 watts with maximum setting of 50 watts) RF energy delivery, and further provide multiplexing capabilities (across a maximum of 30 channels).

Once deployed, the first and second segments 322, 324 contact and conform to a shape of the respective locations, including conforming to and complementing shapes of one or more anatomical structures at the respective locations. In turn, the first and second segments 322, 324 become accurately positioned within the nasal cavity to subsequently deliver, via one or more electrodes 336, precise and focused application of RF thermal energy or non-thermal energy to the one or more target sites to thereby therapeutically modulate associated neural tissue. More specifically, the first and second segments 322, 324 have shapes and sizes when in the expanded configuration that are specifically designed to place portions of the first and second segments 322, 324, and thus one or more electrodes associated therewith 336, into contact with target sites within nasal cavity associated with postganglionic parasympathetic fibers that innervate the nasal mucosa.

For example, the first set of flexible support elements of the first segment 322 conforms to and complements a shape of a first anatomical structure at the first location when the first segment 322 is in the deployed configuration and the second set of flexible support elements of the second segment 124 conforms to and complements a shape of a second anatomical structure at the second location when the second segment is in the deployed configuration. The first and second anatomical structures may include, but are not limited to, inferior turbinate, middle turbinate, superior turbinate, inferior meatus, middle meatus, superior meatus, pterygopalatine region, pterygopalatine fossa, sphenopalatine foramen, accessory sphenopalatine foramen(ae), and sphenopalatine micro-foramen(ae).

In some embodiments, the first segment 322 of the multi-segment end effector 314 is configured in a deployed configuration to fit around at least a portion of a middle turbinate at an anterior position relative to the middle turbinate and the second segment 324 of the multi-segment end effector is configured in a deployed configuration to contact a plurality of tissue locations in a cavity at a posterior position relative to the middle turbinate.

For example, the first set of flexible support elements of the first segment (i.e., struts 330 and 332) conforms to and complements a shape of a lateral attachment and posterior-inferior edge of the middle turbinate when the first segment 322 is in the deployed configuration and the second set of flexible support elements (i.e., struts 334) of the second segment 324 contact a plurality of tissue locations in a cavity at a posterior position relative to the lateral attachment and posterior-inferior edge of middle turbinate when the second segment 324 is in the deployed configuration. Accordingly, when in the deployed configuration, the first and second segments 322, 324 are configured to position one or more associated electrodes 336 at one or more target sites relative to either of the middle turbinate and the plurality of tissue locations in the cavity behind the middle turbinate. In turn, electrodes 336 are configured to deliver RF energy at a level sufficient to therapeutically modulate postganglionic parasympathetic nerves innervating nasal mucosa at an innervation pathway within the nasal cavity of the patient.

As illustrated in FIG. 5E, the first segment 322 comprises a bilateral geometry. In particular, the first segment 322 includes two identical sides, including a first side formed of struts 330a, 332a and a second side formed of struts 330b, 332b. This bilateral geometry allows at least one of the two sides to conform to and accommodate an anatomical structure within the nasal cavity when the first segment 322 is in an expanded state. For example, when in the expanded state, the plurality of struts 330a, 332a contact multiple locations along multiple portions of the anatomical structure and electrodes provided by the struts are configured to emit energy at a level sufficient to create multiple micro-lesions in tissue of the anatomical structure that interrupt neural signals to mucus producing and/or mucosal engorgement elements. In particular, struts 330a, 332a conform to and complement a shape of a lateral attachment and posterior-inferior edge of the middle turbinate when the first segment 322 is in the deployed configuration, thereby allowing for both sides of the anatomical structure to receive energy from the electrodes. By having this independence between first and second side (i.e., right and left side) configurations, the first segment 322 is a true bilateral device. By providing a bilateral geometry, the multi-segment end effector 314 does not require a repeat use configuration to treat the other side of the anatomical structure, as both sides of the structure are accounted at the same time due to the bilateral geometry. The resultant micro-lesion pattern can be repeatable and is predictable in both macro element (depth, volume, shape parameter, surface area) and can be controlled to establish low to high effects of each, as well as micro elements (the thresholding of effects within the range of the macro envelope can be controlled), as well be described in greater detail herein. The systems of the present invention are further able to establish gradients within allowing for control over neural effects without having widespread effect to other cellular bodies, as will be described in greater detail herein.

FIG. 7 is a cross-sectional view of a portion of the shaft 116 of the handheld device taken along lines 7-7 of FIG. 3. As illustrated, the shaft 116 may be constructed from multiple components so as to have the ability to constrain the end effector in the retracted configuration (i.e., the low-profile delivery state) when the end effector is retracted within the shaft 116, and to further provide an atraumatic, low profile and durable means to deliver the end effector to the target site. The shaft 116 includes coaxial tubes which travel from the handle 118 to a distal end of the shaft 116. The shaft 116 assembly is low profile to ensure adequate delivery of therapy in areas requiring low-profile access. The shaft 116 includes an outer sheath 138, surrounding a hypotube 140, which is further assembled over electrode wires 129 which surround an inner lumen 142. The outer sheath 138 serves as the interface between the anatomy and the device 102. The outer sheath 138 may generally include a low friction PTFE liner to minimize friction between the outer sheath 138 and the hypotube 140 during deployment and retraction. In particular the outer sheath 138 may generally include an encapsulated braid along a length of the shaft 116 to provide flexibility while retaining kink resistance and further retaining column and/or tensile strength. For example, the outer sheath 138 may include a soft Pebax material, which is atraumatic and enables smooth delivery through a passageway.

The hypotube 140 is assembled over the electrode wires starting within the handle 118 and travelling to the proximal end of the end effector. The hypotube 140 generally acts to protect the wires during delivery and is malleable to enable flexibility without kinking to thereby improve trackability. The hypotube 140 provides stiffness and enables torqueability of the device 102 to ensure accurate placement of the end effector 314. The hypotube 140 also provides a low friction exterior surface which enables low forces when the outer sheath 138 moves relative to the hypotube 140 during deployment and retraction or constraint. The shaft 116 may be pre-shaped in such a manner so as to complement a given anatomy (e.g., nasal cavity). For example, the hypotube 140 may be annealed to create a bent shaft 116 with a pre-set curve. The hypotube 140 may include a stainless-steel tubing, for example, which interfaces with a liner in the outer sheath 138 for low friction movement.

The inner lumen 142 may generally provide a channel for fluid extraction during a treatment procedure. For example, the inner lumen 142 extends from the distal end of the shaft 116 through the hypotube 140 and to atmosphere via a fluid line (line 121 of FIG. 3). The inner lumen 142 materials are chosen to resist forces of external components acting thereon during a procedure.

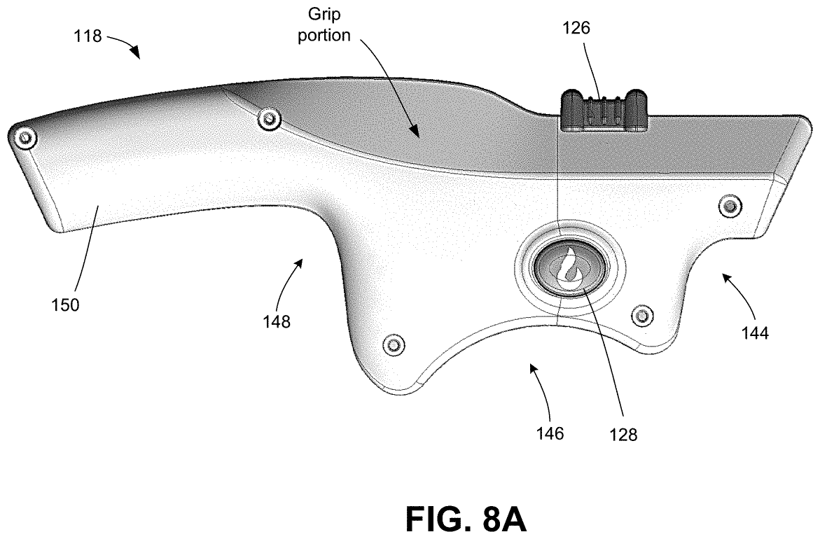

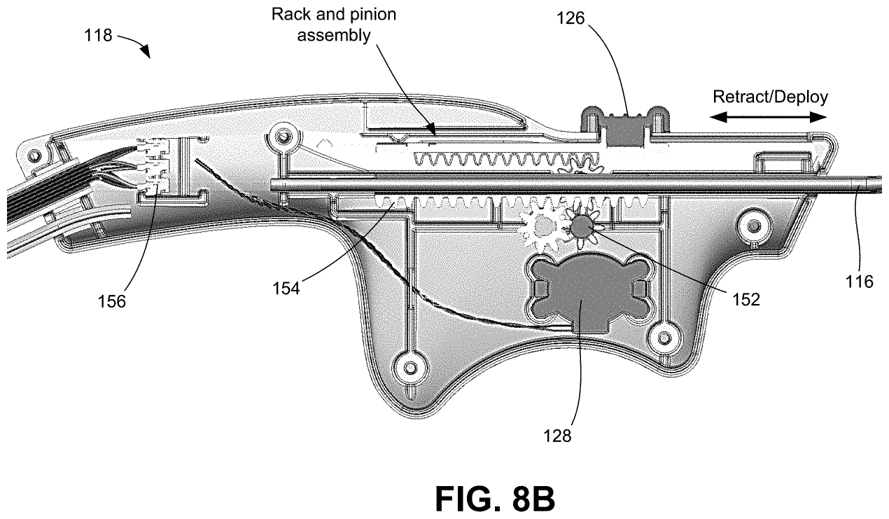

FIG. 8A is a side view of the handle of the handheld 118 and FIG. 8B is a side view of the handle 118 illustrating internal components enclosed within. The handle 118 generally includes an ergonomically-designed grip portion which provides ambidextrous use for both left and right handed use and conforms to hand anthropometrics to allow for at least one of an overhand grip style and an underhand grip style during use in a procedure. For example, the handle 118 may include specific contours, including recesses 144, 146, and 148 which are designed to naturally receive one or more of an operator's fingers in either of an overhand grip or underhand grip style and provide a comfortable feel for the operator. For example, in an underhand grip, recess 144 may naturally receive an operator's index finger, recess 146 may naturally receive an operator's middle finger, and recess 148 may naturally receive an operator's ring and little (pinkie or pinky) fingers which wrap around the proximal protrusion 150 and the operator's thumb naturally rests on a top portion of the handle 118 in a location adjacent to the first mechanism 126. In an overhand grip, the operator's index finger may naturally rest on the top portion of the handle 118, adjacent to the first mechanism 126, while recess 144 may naturally receive the operator's middle finger, recess 146 may naturally receive a portion of the operator's middle and/or ring fingers, and recess 148 may naturally receive and rest within the space (sometimes referred to as the purlicue) between the operator's thumb and index finger.

As previously described, the handle includes multiple user-operated mechanisms, including at least a first mechanism 126 for deployment of the end effector from the collapsed/retracted configuration to the expanded deployed configuration and a second mechanism 128 for controlling of energy output by the end effector, notably energy delivery from one or more electrodes. As shown, the user inputs for the first and second mechanisms 126, 128 are positioned a sufficient distance to one another to allow for simultaneous one-handed operation of both user inputs during a procedure. For example, user input for the first mechanism 126 is positioned on a top portion of the handle 118 adjacent the grip portion and user input for the second mechanism 128 is positioned on side portions of the handle 118 adjacent the grip portion. As such, in an underhand grip style, the operator's thumb rests on the top portion of the handle adjacent to the first mechanism 126 and at least their middle finger is positioned adjacent to the second mechanism 128, each of the first and second mechanisms 126, 128 accessible and able to be actuated. In an overhand grip system, the operator's index finger rests on the top portion of the handle adjacent to the first mechanism 126 and at least their thumb is positioned adjacent to the second mechanism 128, each of the first and second mechanisms 126, 128 accessible and able to be actuated. Accordingly, the handle accommodates various styles of grip and provides a degree of comfort for the surgeon, thereby further improving execution of the procedure and overall outcome.

Referring to FIG. 8B, the various components provided within the handle 118 are illustrated. As shown, the first mechanism 126 may generally include a rack and pinion assembly providing movement of end effector between the retracted and deployed configurations in response to input from a user-operated controller. The rack and pinion assembly generally includes a set of gears 152 for receiving input from the user-operated controller and converting the input to linear motion of a rack member 154 operably associated with at least one of the shaft 116 and the end effector. The rack and pinion assembly comprises a gearing ratio sufficient to balance a stroke length and retraction and deployment forces, thereby improving control over the deployment of the end effector. As shown, the rack member 154 may be coupled to a portion of the shaft 116, for example, such that movement of the rack member 154 in a direction towards a proximal end of the handle 118 results in corresponding movement of the shaft 116 while the end effector remains stationary, thereby exposing the end effector and allowing the end effector to transition from the constrained, retracted configuration to the expanded, deployed configuration. Similarly, movement of the rack member 154 in a direction towards a distal end of the handle 118 results in corresponding movement of the shaft 116 while the end effector remains stationary, and thereby encloses the end effector within the shaft 116. It should be noted that, in other embodiments, the rack member 154 may be directly coupled to a portion of the end effector such that movement of the rack member 154 results in corresponding movement of the end effector while the shaft 116 remains stationary, thereby transitioning the end effector between the retracted and deployed configurations.

The user-operated controller associated with the first mechanism 126 may include a slider mechanism operably associated with the rack and pinion rail assembly. Movement of the slider mechanism in a rearward direction towards a proximal end of the handle results in transitioning of the end effector to the deployed configuration and movement of the slider mechanism in a forward direction towards a distal end of the handle results in transitioning of the end effector to the retracted configuration. In other embodiment, the user-operated controller associated with the first mechanism 126 may include a scroll wheel mechanism operably associated with the rack and pinion rail assembly. Rotation of the wheel in a rearward direction towards a proximal end of the handle results in transitioning of the end effector to the deployed configuration and rotation of the wheel in a forward direction towards a distal end of the handle results in transitioning of the end effector to the retracted configuration.

As previously noted, the console unit 104 is configured to provide an intuitive and automated control and targeting of energy output from the treatment device 102 sufficient to ensure successful treatment of a condition, such as a nasal condition, including rhinosinusitis. In particular, the console unit 104 provides a user, via an interactive interface 112, with comprehensive operational instructions for performing a given procedure and, in response to user input, further provides automatic and precise control over the ablation/modulation of the targeted tissue while minimizing and/or preventing collateral damage to surrounding or adjacent non-targeted tissue at the target site. More specifically, the console unit 104 provides the user with step-by-step guidance, in the form of selectable inputs, for treating, via the treatment device 102, rhinosinusitis. It should be noted, however, that the systems and methods of the present invention can be used to treat various conditions, and is not limited to the treatment of a nasal condition.

Such step-by-step guidance provided via the interactive interface 112 of the console unit 104 may include, for example, directing the user through the initial set up of the device 102 with the console unit 104, including authenticating the device 102 to ensure that the device is in fact suitable and/or authorized to operate with the console unit 104.

In the medical industry, there are many surgical devices, instruments and systems comprised of individual components that must work together properly to ensure treatment is performed safely and as intended. For example, some procedures include the use of a central console or power supply and a working instrument (i.e., a handheld instrument providing direct treatment to the patient) operably associated with the central console or power supply. The instrument is generally a single use device, while the central console or power supply is reusable. Accordingly, prior to beginning a medical procedure, it is important that the proper components be connected to one another. Oftentimes, the manufacturer of a control unit, for example, may recommend usage of particular brands of a working instrument with the console unit. When one of the components being used is not a certified product, the full capabilities of the system may not be achieved and may further cause malfunctions, endangering patient safety. For example use of an instrument can result in damage to the equipment, delay in conducting a medical procedure until the proper instrument is obtained, and/or result in the potential for an ineffective, damaging, or potentially life-threatening medical procedure.

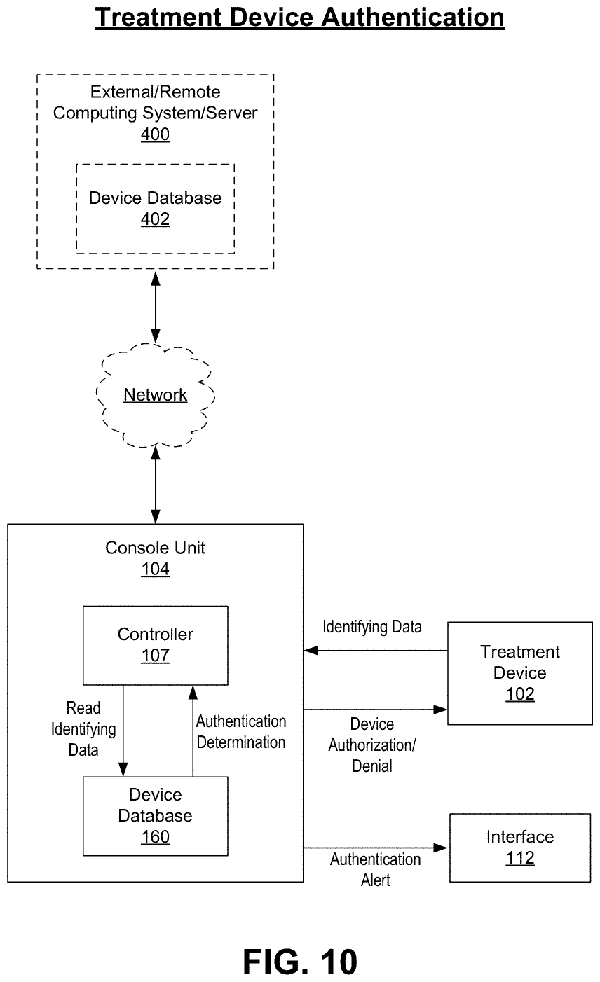

FIG. 9 is a block diagram illustrating the console unit 104 of the present disclosure and authentication of a handheld treatment device 102 to be used with the console unit 104. FIG. 10 is a block diagram illustrating authentication of the treatment device in greater detail.

As illustrated, the console unit 104 configured to be operably associated with a treatment device 102 and control operation thereof as previously described herein. The console unit 104 is configured to analyze identifying data associated with the treatment device 102 upon connection of the treatment device 102 to the console unit and determine authenticity of the treatment device 102 based on the analysis of the identifying data. Upon determining the authenticity of the treatment device 102, the console unit 104 is then configured to output, via the interactive interface 112, an alert to a user (i.e., surgeon, operating staff, or other medical professional) indicating the authenticity determination (i.e., an indication as to whether the device 102 is authentic or not). The alert may include, for example, text displayed on a graphical user interface (GUI) indicating either the compatibility of the treatment device, and further authorize its use for performing a procedure, or the incompatibility of the treatment device and further provide one or more suggested actions. The one or more suggested actions may include a suggestion that the user couple an authentic and compatible treatment device to the console unit.

Accordingly, the system of the present invention ensures that only authorized treatment devices are able to be used with the console unit 104. The authentication ensures that only those treatment devices recommended and authorized by a manufacturer are to be used, thereby ensuring that the treatment system functions as intended and patient safety is maintained. The authentication further protects against the use of counterfeit components. As counterfeit proprietary components become more prevalent, the need to authenticate original products becomes increasingly necessary. By embedding identifying data directly into the treatment device and utilizing reading technology for authentication, manufacturers can foil counterfeiters and secure recurring revenue streams, which may otherwise be lost due to counterfeit products.

Upon connecting the treatment device 102 to the console unit 104, the controller 104 is configured to read identifying data associated with the device 102. For example, the device 102 may include an RFID tag 103 containing identifying data and the console unit may include an RFID reader 158 configured to read identifying data embedded in the RFID tag 103, wherein such RFID tag data is analyzed to determine authenticity of the device 102. The data from the RFID tag is read by the RFID reader, and then analyzed by the controller 107.

A determination is made as to whether the device 102 is authentic (i.e., suitable for use with the console unit 104) based on the authentication analysis. In the event that the device 102 is determined to be authentic, the controller 107 allows for the use of the device 102 in a given procedure (i.e., transmission of energy from the generator 106 to the device 102 and thus a procedure can be performed using the device 102). In the event that the device 102 is determined to not be authentic, the controller 107 prevents transmission of energy to the device 102. Furthermore, upon determining the authenticity of the device 102, the console unit 104 is configured to provide an alert, via the interface 112, indicating the authenticity determination. In particular, the console unit 104 is configured to output, via the interface 112, at least one of audible alert and visual alert indicating to the user whether the treatment device 102 is authentic or inauthentic.

The analysis of the identifying data comprises correlating the identifying data with authentication data. The authentication data may include a unique identifier including an authentication key or identity number associated with authentic treatment devices permitted to be used with the console unit. The treatment device is determined to be authentic upon a positive correlation and determined to be inauthentic upon a negative correlation.

In the event that the device 102 is determined to be inauthentic, and thus incompatible with the console unit 104, the console unit is configured to prevent use of the device 102 (i.e., prevent transmission of energy from the generator 106 to the device 102) and output an audible or visual alert, via the interface 112, to the user indicating the inauthenticity of the device 102. The alert may include a particular audible tone and/or text displayed on the interface 112 indicating the inauthenticity of the treatment device 102 (i.e., a first tone associated with inauthenticity and/or text in the form of a message and/or a first color indicative of inauthenticity, such as red) and further provide one or more suggested actions. The one or more suggested actions may include, for example, a suggestion that the user couple an authentic treatment device to the console unit.

In the event that the device 102 is determined to be authentic, the console unit 104 may then determine whether there has been any prior use of the treatment device 102, including whether such prior use was associated with the console unit 104 or a different console unit, based on the analysis of the identifying data. Upon a determination that the treatment device 102 is unused, the console unit 104 outputs, via the interactive interface 112, an alert to the user indicating that the treatment device 102 is set for use (i.e., permits the user to advance to the next operational options provided via the step-by-step guidance, including initiation of energy delivery). The alert may include a particular audible tone and/or text displayed on the interface 112 indicating the authenticity of the treatment device 102 (i.e., a second tone associated with authenticity and/or text in the form of a message and/or a second color indicative of inauthenticity, such as green) and allow the user to advance to the next operational options provided via the step-by-step guidance to begin a given procedure.

Upon a determination that the treatment device 102 has prior use and such prior use was associated with the console unit 104, the console unit 104 is configured to determine an amount and/or timeframe of the prior use, based on the analysis of the identifying data. Upon a determination that the prior use was within a predetermined grace period, the console unit 104 is configured to output, via the interactive interface 112, an alert to the user indicating that the treatment device 102 is set for use and further permit use of the device 102. Again, the alert may include a particular audible tone and/or text displayed on the interface 112 indicating the authenticity of the treatment device 102 (i.e., a second tone associated with authenticity and/or text in the form of a message and/or a second color indicative of inauthenticity, such as green) and allow the user to advance to the next operational options provided via the step-by-step guidance to begin a given procedure. Upon a determination that the prior use with outside of the predetermined grace period, the console unit 104 is configured to prevent use of the device 102 and output, via the interactive interface 112, at least one of audible alert and visual alert indicating to the user that the treatment device 102 is expired and further prevents use of the device 102. Again, the alert may include a particular audible tone and/or text displayed on the interface 112 indicating the incompatibility of the treatment device 102 (i.e., the first tone associated with inauthenticity/incompatibility and/or text in the form of a message and/or a first color indicative of inauthenticity/incompatibility, such as red) and further provide one or more suggested actions. The one or more suggested actions may include, for example, a suggestion that the user couple an authentic treatment device to the console unit.

Upon a determination that the treatment device 102 has been previously used and such prior use was associated with a different console unit, the console unit 104 is configured to output an alert indicating to the user that the treatment device 102 is incompatible with the console unit 104 and further prevents use of the device 102. Again, the alert may include a particular audible tone and/or text displayed on the interface 112 indicating the inauthenticity of the treatment device 102 (i.e., the first tone associated with inauthenticity/incompatibility and/or text in the form of a message and/or a first color indicative of inauthenticity/incompatibility, such as red) and further provide one or more suggested actions, including a suggestion that the user couple an authentic treatment device to the console unit.

The controller 107 may include software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices. “Circuitry”, as used in any embodiment herein, may comprise, for example, singly or in any combination, hardwired circuitry, programmable circuitry such as computer processors comprising one or more individual instruction processing cores, state machine circuitry, and/or firmware that stores instructions executed by programmable circuitry. For example, the controller 107 may include a hardware processor coupled to non-transitory, computer-readable memory containing instructions executable by the processor to cause the controller to carry out various functions of the system 100 as described herein, including controlled energy output.

The authentication analysis may be based on a correlation of the identifying data with known, predefined authentication data stored in a database, either a local database (i.e., device database 160) forming part of the console unit 104, or a remote database hosted via a remote server 400 (i.e., device database 402). For example, in some embodiments, the console unit 104 may communicate and exchange data with a remote server 400 over a network. The network may represent, for example, a private or non-private local area network (LAN), personal area network (PAN), storage area network (SAN), backbone network, global area network (GAN), wide area network (WAN), or collection of any such computer networks such as an intranet, extranet or the Internet (i.e., a global system of interconnected network upon which various applications or service run including, for example, the World Wide Web).

The known, predefined authentication data stored in the database (database 160 or database 402) may be controlled by the owner/manufacturer of the console unit 104, for example, such that the owner/manufacturer can determine what treatment devices are to be used with the console unit. For example, the owner/manufacturer may set a specific authentication key or provide for specific identity numbers that are proprietary to the owner/manufacturer. As such, the identifying data for any given treatment device must include a corresponding unique identifier (i.e., authentication key or identity number) in order to be deemed authentic. It should be further noted that the device database 160, 402 may include a profile for authorized devices (i.e., devices deemed to be authentic and compatible with the console unit 104), wherein the profile of a given device may include, in addition to authentication data, may include operational history of a given device, such as any prior use of the device, including length of use (i.e., elapsed time of use, number of uses, etc.) and specific console units to which the device has been previously connected and used, and the like).

One approach to uniquely identifying a treatment device is to authenticate the device by using a private key. In such an approach, both the console unit 104 and the tag 103 are taught an identical key. The RFID tag 103 and console unit 104 then operate in conjunction to authenticate the key. More specifically, the console unit 104 generates a random, unique challenge number. The RFID tag 103 uses this challenge, in combination with the key to generate a response of an authentication code. The method for generating this code (known as a hash function) masks the value of the key. Another approach to uniquely identifying a laser probe is to use unique and unchangeable identity numbers. This approach can be used if there is a region of memory (e.g., a serial or model number), that can only be written by the RFID manufacturer. The protection is realized by ensuring that the manufacturer only provides tags with legal identification numbers, which prevents simple duplication of legitimate tags.

The identifying data may include other information and/or characteristics associated with the device 102. For example, in some embodiments, the identifying data further includes operational history information of the device. As such, in some embodiments, it is further possible to utilize the controller 107 to deauthenticate a device 102 based on operational history, such as in the event that the device has already been used, if it has been used with the current console unit or a different console unit, and/or reached the suggested maximum number of uses, thereby preventing further use of the device with the console unit.

It should further be noted that other forms of authentication can be used. For example, in addition, or alternatively, to user programmable sets of authentication data (i.e., unique identifiers including an authentication key and/or identity number), systems and methods of the present invention may include challenge-response authentication protocols. For example, the interface may present an operator with a question (“challenge”), to which the operator must provide a valid answer (“response”) to be authenticated. The simplest example of a challenge-response protocol is password authentication, where the challenge is asking for the password and the valid response is the correct password. However, other, more complicated versions of challenge-response protocols may be used. Additional cryptographic techniques may be used, such as a message authentication code (MAC) protocols. Sometimes known as a tag, a MAC is a short piece of information used to authenticate a message, so as to confirm that the message came from the stated sender (its authenticity) and has not been changed.

Upon authenticating the device 102, the step-by-step guidance provided via the interactive interface 112 of the console unit 104 further directs the user to select a location in which to provide treatment. For example, if the given procedure involves treating a nasal condition, such as rhinosinusitis, the user may be directed to select one of the nasal cavities in which to apply treatment (i.e., left or right nasal cavity). Based on the user's selection of a given nasal cavity, the console unit further provides the user with an indication as to when the device is primed and ready to perform treatment in the selected location. In particular, the console unit 104 is configured to perform an assessment of one or more electrodes associated with an end effector of the treatment device, wherein such assessment includes a determination of whether electrodes are available for use (i.e., via an impedance assessment of each electrode).

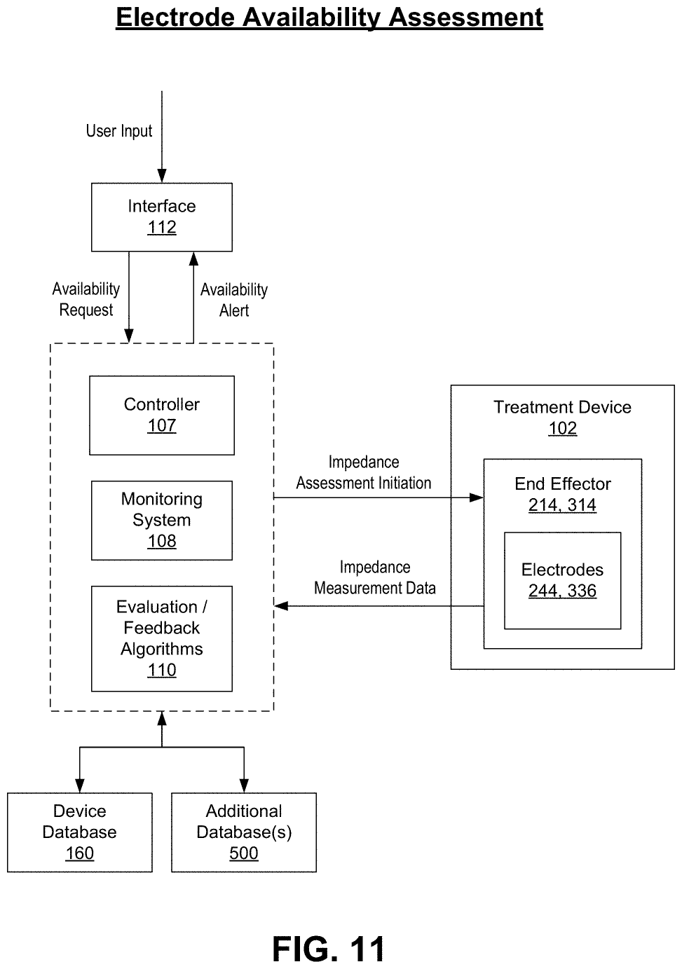

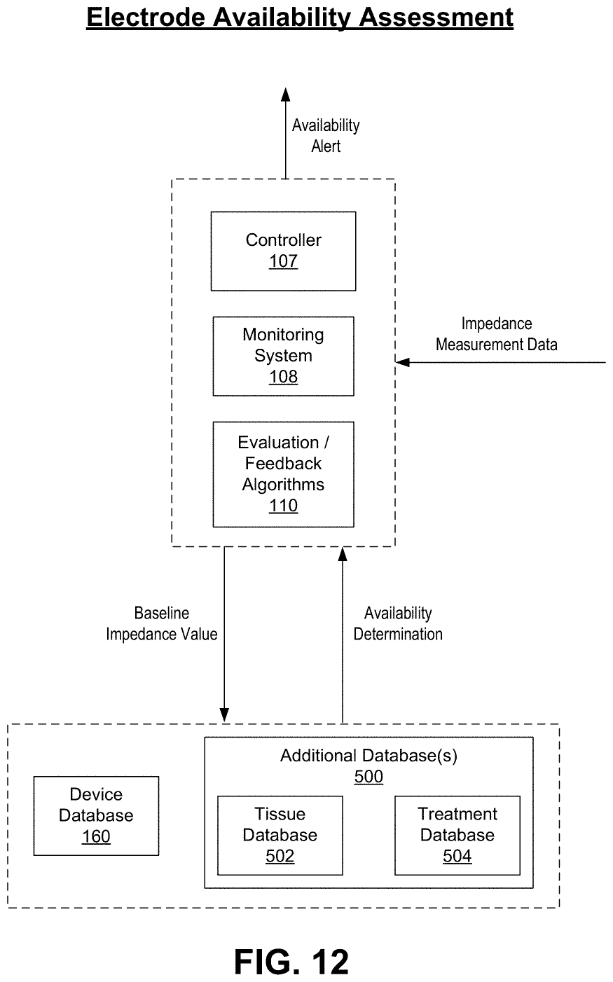

FIG. 11 is a block diagram illustrating an availability assessment of one or more electrodes of an end effector of a handheld treatment device consistent with the present disclosure. FIG. 12 is a block diagram illustrating the availability assessment in greater detail.

Upon a user selecting, via the interactive interface 112, a particular cavity in which to initiate treatment, the user is then directed to an availability assessment portion of the operational procedures for carrying out treatment. In particular, prior to delivery of any energy to targeted tissue, an impedance check must first be performed to determine which electrodes are available to deliver energy. The console unit 104 (via the controller 107, monitoring system 108, and evaluation/feedback algorithms 110) is configured to receive, via user input with the interface 112, a request for a determination of availability of the one or more electrodes for applying treatment to one or more target sites within a selected one of a left side and a right side of the sino-nasal cavity of the patient. Upon receiving such request, the console unit 104 (via the controller 107, monitoring system 108, and evaluation/feedback algorithms 110) is configured to initiate an impedance assessment of the one or more electrodes and further output, via the interactive interface 112, an alert to the user indicating a determined availability of the one or electrodes based on the impedance assessment.

In particular, initiating the impedance assessment includes receiving, from the one or more electrodes, impedance measurement data associated with tissue at the one or more target sites within the selected one of the left and right sides of the sino-nasal cavity. The impedance measurement data is collected via techniques previously described herein. The console unit 104 (via the controller 107, monitoring system 108, and evaluation/feedback algorithms 110) is configured to process the impedance measurement data to calculate a baseline impedance value for each of the one or more electrodes. The processing of the impedance measurement data generally includes calculating aggregate impedance values across a set of multiple pairs of the electrodes within a selected one of the left and right sides of the sino-nasal cavity. It should be noted that the console unit 104 (via the controller 107, monitoring system 108, and evaluation/feedback algorithms 110) is configured to process impedance measurement data of all pairs of electrodes of the set within the selected one of the left and right sides of the sino-nasal cavity.

The determined availability of the one or more pairs of the electrodes is based on a comparison of the calculated baseline impedance value with a predetermined range of baseline impedance values. In some embodiments, the predetermined range of baseline impedance values includes, for example, a low baseline impedance value of approximately 100 ohms and a high baseline impedance value of approximately 1 kohms. Yet still, in some embodiments, the predetermined range of baseline impedance values includes a low baseline impedance value of approximately 400 ohms and a high baseline impedance value of approximately 700 ohms. The predetermined range of baseline impedance values may be stored in one or more databases (additional databases 500) and be associated with the particular tissue type to undergo treatment (i.e., tissue database 502) and/or a particular treatment plan controlling delivery of energy to targeted tissue (i.e., treatment database 504). Accordingly, the console unit 104 (via the controller 107, monitoring system 108, and evaluation/feedback algorithms 110) to compare the calculated baseline impedance value with the predetermine range of baseline impedance values (stored in one or more databases 500).

As previously described herein, the device 102 may include a multi-segmented end effector (i.e., end effector 314), and thus may comprise a plurality of support structures (also referred to herein as a “leaflet pair”) that each comprise one or more electrodes.

A pair of the plurality of support structures is determined to be available for applying treatment, via one or more associated electrodes, to one or more target sites when the calculated baseline value falls within the predetermined range of baseline impedance values. Upon a determination that a given pair of support structures is available for applying treatment, the console unit 104 is configured to output at least one of audible alert and visual alert, via the interactive interface, indicating the availability. For example, the interface may display each leaflet pair and provide a visual indication of the availability by way of color coding. For example, the interface may display a given leaflet pair, determined to be available for use in treatment, in a first color, such as blue. The console unit 104 is configured to cycle through all leaflet pairs and perform an availability assessment on each (i.e., a determination of which leaflet pairs are available and unavailable).

Once all leaflet pairs have undergone an availability assessment, the console unit 104 is further configured to determine whether at least a minimum required number of pairs of the plurality of support structures are available. In the event that a minimum required number of pairs of the support structures are available, the console unit 104 is configured to output at least one of audible alert and visual alert, via the interactive interface, indicating to the user that the treatment device 102 is ready to provide treatment and further permit transmission of energy to the one or more electrodes for subsequent targeted delivery of energy to one or more target sites within the selected one of the left and right sides of the sino-nasal cavity. The visual alert may include at least one of text and the first color (e.g., blue) displayed on the interface 112 indicating the availability of one or more pairs of the plurality of support structures. The text, for example, may be in the form of a message indicating that the device 102 is ready to perform treatment and provide suggested action to the user as to have to initiate activation of the available leaflet pairs. In the event that the minimum required number of leaflet pairs is unavailable, the console unit 104 is configured to continue cycling through leaflet pairs and performing the above described availability assessment on each.

A pair of the plurality of support structures is determined to be unavailable for applying treatment, via one or more associated electrodes, to one or more target sites when the calculated baseline value falls outside the predetermined range of baseline impedance values. In turn, the console unit 104 prevents transmission of energy from an energy source (i.e., generator 106) to one or more electrodes associated within a pair of the plurality of support structures determined to be unavailable. Upon a determination that a given pair of support structures is unavailable for applying treatment, the console unit 104 is configured to output at least one of audible alert and visual alert, via the interactive interface, indicating the unavailability. For example, the interface may display a given leaflet pair, determined to be unavailable for use in treatment, in a second color, such as gray.

It should be noted that, the console unit 104 is further configured to permit repositioning of a pair of the plurality of support structures determined to be unavailable when the calculated baseline value falls outside the predetermined range of baseline impedance values. In particular, a user may receive the visual alert (i.e., a gray colored leaflet pair) and, in turn, reposition the end effector 214, 314, at which point the availability assessment is performed again for that given leaflet pair.

It should be noted that the calculated baseline impedance value for a given leaflet pair may be stored within a respective profile of a treatment device 102 (stored within device database 160). Accordingly, such data for a given device may be readily available for processing, if needed, during the targeted energy delivery portion of the procedure. It should be further noted that the tissue type data (stored in tissue database 502) and the treatment data (stored in treatment database 504) may further be tied to a given device and thus correlated with device data (stored in device database 160).

Depending on the availability of one or more electrodes for energy delivery (including availability of specific leaflet pairs), the user may be presented with operational inputs, including the option of initiating treatment.

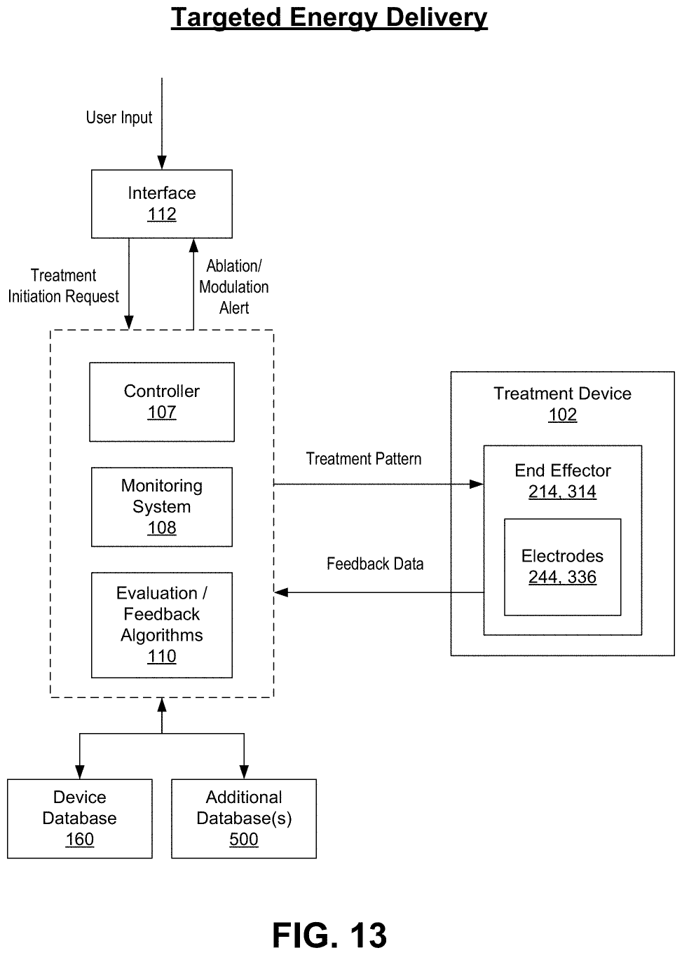

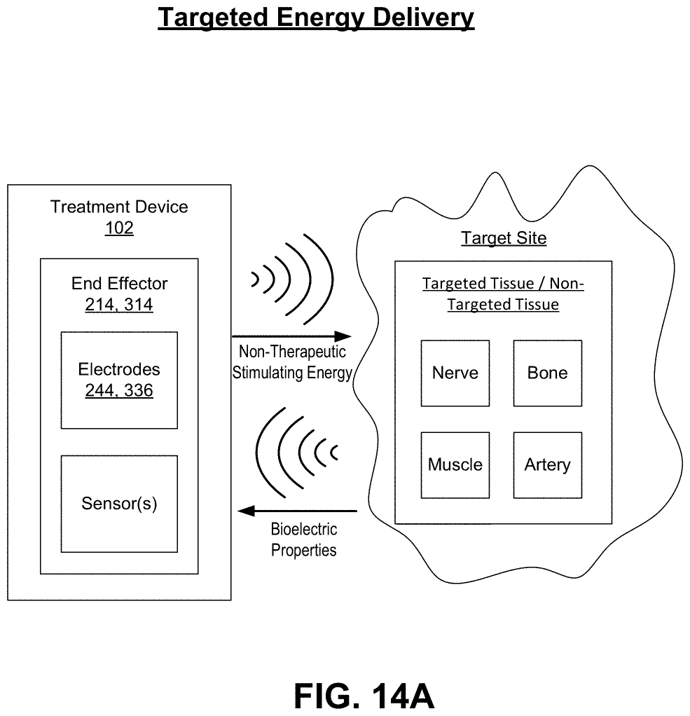

FIG. 13 is a block diagram illustrating controlled and targeted energy delivery from one or more electrodes of an end effector of the treatment device via the console unit based on a calculated treatment pattern.

Upon receiving user selection of treatment initiation, the console unit 104 (via the controller 107, monitoring system 108, and evaluation/feedback algorithms 110) is configured to determine a specific treatment pattern for controlling delivery of energy at a specific level for a specific period of time to the tissue of interest (i.e., the targeted tissue) sufficient to ensure successful ablation/modulation of the targeted tissue while minimizing and/or preventing collateral damage to surrounding or adjacent non-targeted tissue at the target site.

In particular, the console unit 104 receives, via user input with the interactive interface 112, a request to initiate treatment of a selected one of a left side and a right side of the sino-nasal cavity of the patient. In turn, the console unit 104 identifies one or more sets of support structures (leaflet pairs) to be activated for treating the selected one of the left and right side of the sino-nasal cavity. The console unit 104 further calculates a treatment pattern for controlling delivery of energy from electrodes associated with each leaflet pair of a given identified set and further receives feedback data associated with each leaflet pair upon supplying treatment energy to respective electrodes. The console unit 104 processes the feedback data to determine a status of each leaflet pair with respect to the treatment pattern. The status includes, for example, an incomplete state, a successful state, and an unsuccessful state. An incomplete state generally refers to a leaflet pair as still in-progress with respect to the treatment pattern (e.g., the leaflet pair is currently receiving, or awaiting receipt of, RF energy from generator 106 for delivery to targeted tissue). A successful state generally refers to a leaflet pair achieving the desired characteristic event and subsequent treatment of the targeted tissue (i.e., successful ablation/modulation of the targeted tissue). An unsuccessful state generally refers to a leaflet pair not achieving the desired characteristic event and thus remains available for further energy delivery. The console unit 104 is further configured to output, via the interactive interface 112, an alert to a user indicating a status of each leaflet pair. In particular, the console unit 104 is configured to output at least a visual alert indicating a status of each leaflet pair of a given set of leaflet pairs. In particular, the interface may display each leaflet pair of a given set and provide a specific color coding indicative of a status of each leaflet pair. For example, a leaflet pair having either an incomplete status or an unsuccessful status may be displayed in a first color (e.g., blue), while a leaflet pair having a successful status may be displayed in a second contrasting color (e.g., green). It should be noted that, as a leaflet pair is delivering energy, the color coding may gradually change from a first color to a second color as the status changes. For example, the status of a given leaflet pair may be provided and updated in real-time via the interface, such that a leaflet pair that is currently in an incomplete state (and displayed in a blue color) may gradually reach a successful state over an elapsed period of time (and turn from a blue color to a green color on the interface). The alert may further include text indicating the specific state of a given leaflet pair.

The treatment pattern (which essentially controls delivery of energy from the end effector to the targeted tissue) is based, at least in part, on determined types of tissue(s) at the target site.

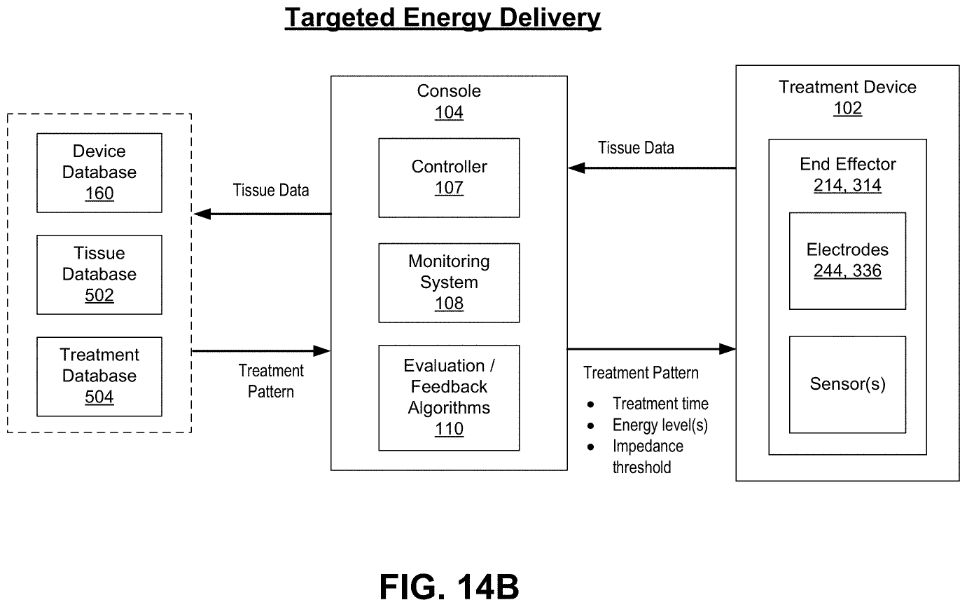

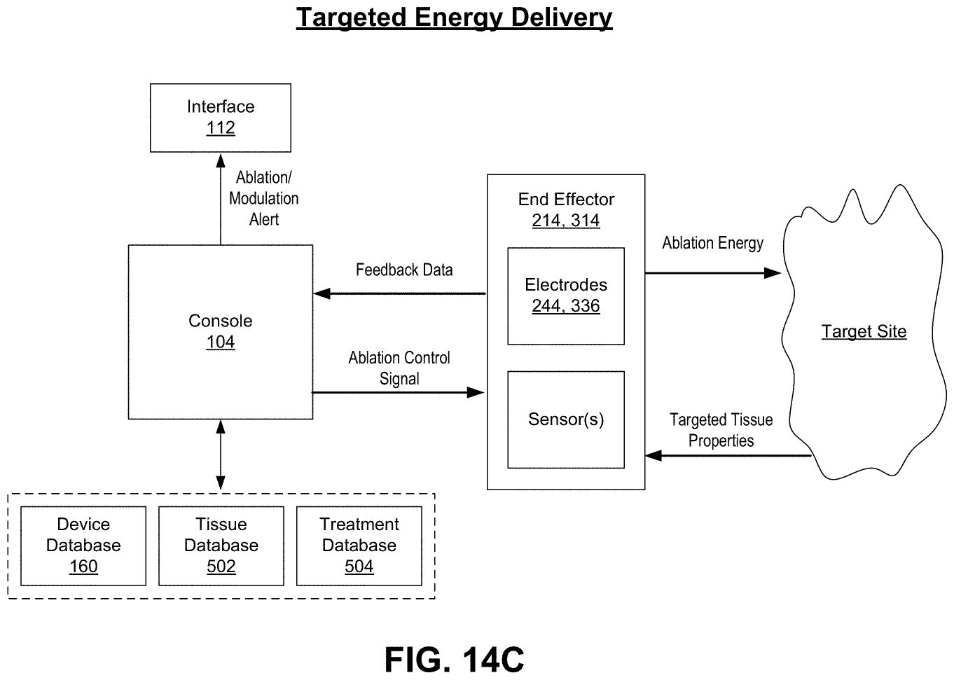

FIGS. 14A, 14B, and 14C are block diagrams illustrating the process of sensing, via an end effector, data associated with one or more tissues at a target site, notably bioelectric properties of one more tissues at the target site, and the subsequent processing of such data (via the controller 107, monitoring system 108, and evaluation/feedback algorithms 110) to determine the type of tissue(s) at the target site, determining a treatment pattern to be delivered by one or more of the plurality of electrodes of the end effector based on identified tissue types (as well as tissue location and/or depth), and subsequent receipt and processing of real-time feedback data associated with the targeted tissue undergoing treatment. The ablation energy associated with the ablation pattern is at a level sufficient to ablate a targeted tissue and minimize and/or prevent collateral damage to surrounding or adjacent non-targeted tissue at the target site.

Block diagrams of FIGS. 14A, 14B, and 14C include reference to both end effectors 214, 314. For ease of description, the following process describes the use of end effector 214. However, end effectors 214 and 314 are similarly configured with respect to sensing data associated with at least the presence of neural tissue and other properties of the neural tissue, including neural tissue depth. Accordingly, the following process is not limited to end effector 214.

FIG. 14A is a block diagram illustrating delivery of non-therapeutic energy from electrodes 244 of the end effector at a frequency for sensing one or more properties associated with tissue at a target site in response to the non-therapeutic energy.

As previously described, the handheld treatment device includes an end effector comprising a micro-electrode array arranged about a plurality of struts. For example, end effector 214 includes a plurality of struts 240 that are spaced apart from each other to form a frame or basket 242 when the end effector 214 is in the expanded state. The struts 240 include a plurality of energy delivery elements, such as a plurality of electrodes 244. In the expanded state, each of the plurality of struts is able to conform to and accommodate an anatomical structure at a target site. When positioned, the struts may contact multiple locations along multiple portions of a target site and thereby position one or more electrodes 244 against tissue at a target site. At least a subset of electrodes is configured to deliver non-therapeutic stimulating energy at a frequency/waveform to respective positions at the target site to thereby sense the bioelectric properties of the one or more tissues at the target site, and further convey such data to the console 104. In addition to bioelectric properties, the data may also include at least one of physiological properties and thermal properties of tissue at the target site.

For example, upon delivering non-therapeutic stimulating energy (via one or more electrodes 244) to respective positions, various properties of the tissue at the one or more target sites can be detected. This information can then be transmitted to the console 104, particularly the controller 107, monitoring system 108, and evaluation/feedback algorithms 110 to determine the anatomy at the target site (e.g., tissue types, tissue locations, vasculature, bone structures, foramen, sinuses, etc.), locate a tissue of interest (targeted tissue to receive electric therapeutic stimulation), such as neural tissue, differentiate between different types of neural tissue, and map the anatomical and/or neural structure at the target site. For example, the end effector 214 can be used to detect resistance, complex electrical impedance, dielectric properties, temperature, and/or other properties that indicate the presence of neural fibers and/or other anatomical structures in the target region. In certain embodiments, the end effector 214, together with the console 104 components, can be used to determine resistance (rather than impedance) of the tissue (i.e., the load) to more accurately identify the characteristics of the tissue. For example, the evaluation/feedback algorithms 110 can determine resistance of the tissue by detecting the actual power and current of the load (e.g., via the electrodes 244).

In some embodiments, the system 100 provides resistance measurements with a high degree of accuracy and a very high degree of precision, such as precision measurements to the hundredths of an Ohm (e.g., 0.01Ω) for the range of 1-50Ω. The high degree of resistance detection accuracy provided by the system 100 allows for the detection sub-microscale structures, including the firing of neural tissue, differences between neural tissue and other anatomical structures (e.g., blood vessels), and even different types of neural tissue. This information can be analyzed by the evaluation/feedback algorithms 110 and/or the controller 107 and communicated to the operator via a high resolution spatial grid (e.g., on the display 112) and/or other type of display to identify neural tissue and other anatomy at the treatment site and/or indicate predicted neuromodulation regions based on the ablation pattern with respect to the mapped anatomy.

As previously described, in certain embodiments, each electrode 244 can be operated independently of the other electrodes 244. For example, each electrode can be individually activated and the polarity and amplitude of each electrode can be selected by an operator or a control algorithm executed by the controller 107. The selective independent control of the electrodes 244 allows the end effector 214 to detect information (i.e., the presence of neural tissue, depth of neural tissue, and other physiological and bioelectrical properties) and subsequently deliver RF energy to highly customized regions. For example, a select portion of the electrodes 244 can be activated to target specific neural fibers in a specific region while the other electrodes 244 remain inactive. In addition, the electrodes 244 can be individually activated to stimulate or therapeutically modulate certain regions in a specific pattern at different times (e.g., via multiplexing), which facilitates detection of anatomical parameters across a zone of interest and/or regulated therapeutic neuromodulation.

As previously described, the system 100 can identify tissue type of one or more tissues at a target site prior to therapy such that the therapeutic stimulation can be applied to precise regions including targeted tissue, while avoiding negative effects on non-targeted tissue and structures (e.g., blood vessels). For example, the system 100 can detect various bioelectrical parameters in an interest zone to determine the location and morphology of various tissue types (e.g., different types of neural tissue, neuronal directionality, etc.) and/or other tissue (e.g., glandular structures, vessels, bony regions, etc.). The system 100 is further configured to measure bioelectric potential.

To do so, one or more of the electrodes 244 is placed in contact with an epithelial surface at a region of interest (e.g., a treatment site). Electrical stimuli (e.g., constant or pulsed currents at one or more frequencies, and/or alternating (sine, square, triangle, sawtooth, etc.) wave or direct constant current/power/voltage source at one or more frequencies) are applied to the tissue by one or more electrodes 244 at or near the treatment site, and the voltage and/or current differences based on the wave applied at various different frequencies between various pairs of electrodes 244 of the end effector 214 may be measured to produce a spectral profile or map of the detected bioelectric potential, which can be used to identify different types of tissues (e.g., vessels, neural tissue, and/or other types of tissue) in the region of interest. For example, a fixed current (i.e., direct or alternating current) can be applied to a pair of electrodes 244 adjacent to each other and the resultant voltages and/or currents between other pairs of adjacent electrodes 244 are measured. Conversely, a fixed voltage (i.e. mono or bi-phasic) can be applied to a pair of electrodes 244 adjacent to each other and the resultant current between other pairs of adjacent electrodes 244 are measured. It will be appreciated that the current injection electrodes 244 and measurement electrodes 244 need not be adjacent, and that modifying the spacing between the two current injection electrodes 244 can affect the depth of the recorded signals. For example, closely-spaced current injection electrodes 244 provided recorded signals associated with tissue deeper from the surface of the tissue than further spaced apart current injection electrodes 244 that provide recorded signals associated with tissue at shallower depths. Recordings from electrode pairs with different spacings may be merged to provide additional information on depth and localization of anatomical structures.

Further, complex impedance and/or resistance measurements of the tissue at the region of interest can be detected directly from current-voltage data provided by the bioelectric potential measurements while differing levels of frequency currents are applied to the tissue (e.g., via the end effector 114), and this information can be used to map the neural and anatomical structures by the use of frequency differentiation reconstruction. In particular, current-voltage data may be observed with the difference in dielectric and conductive properties of tissue type when different levels of current frequencies are applied. Applying the stimuli at different frequencies will target different stratified layers or cellular bodies or clusters. At high signal frequencies (e.g., electrical injection or stimulation), for example, cell membranes of the neural tissue do not impede current flow, and the current passes directly through the cell membranes. In this case, the resultant measurement (e.g., impedance, resistance, capacitance, and/or induction) is a function of the intracellular and extracellular tissue and liquids. At low signal frequencies, the membranes impede current flow to provide different defining characteristics of the tissues, such as the shapes of the cells or cell spacing. The stimulation frequencies can be in the megahertz range, in the kilohertz range (e.g., 400-500 kHz, 450-480 kHz, etc.), and/or other frequencies attuned to the tissue being stimulated and the characteristics of the device being used. The detected complex impedance or resistances levels from the zone of interest can be displayed to the user (e.g., via the display 112) to visualize certain structures based on the stimulus frequency.

Further, the inherent morphology and composition of the anatomical structures within a given region or zone of a patient's body react differently to different frequencies and, therefore, specific frequencies can be selected to identify very specific structures. For example, the morphology or composition of targeted structures for anatomical mapping may depend on whether the cells of tissue or other structure are membranonic, stratified, and/or annular. In various embodiments, the applied stimulation signals can have predetermined frequencies attuned to specific neural tissue, such as the level of myelination and/or morphology of the myelination. For example, second axonal parasympathetic structures are poorly myelinated than sympathetic nerves or other structures and, therefore, will have a distinguishable response (e.g., complex impedance, resistance, etc.) with respect to a selected frequency than sympathetic nerves. Accordingly, applying signals with different frequencies to the target site can distinguish the targeted parasympathetic nerves from the non-targeted sensory nerves, and therefore provide highly specific target sites for neural mapping before or after therapy and/or neural evaluation post-therapy.

In some embodiments, the neural and/or anatomical mapping includes measuring data at a region of interest with at least two different frequencies to identify certain anatomical structures such that the measurements are taken first based on a response to an injection signal having a first frequency and then again based on an injection signal having a second frequency different from the first. For example, there are two frequencies at which hypertrophied (i.e., disease-state characteristics) sub-mucosal targets have a different electrical conductivity or permittivity compared to “normal” (i.e., healthy) tissue. Complex conductivity may be determined based on one or more measured physiological parameters (e.g., complex impedance, resistance, dielectric measurements, dipole measurements, etc.) and/or observance of one or more confidently known attributes or signatures. Furthermore, the system 100 can also apply neuromodulation energy via the electrodes 244 at one or more predetermined frequencies attuned to a target neural structure to provide highly targeted ablation of the selected neural structure associated with the frequency(ies). This highly targeted neuromodulation also reduces the collateral effects of neuromodulation therapy to non-target sites/structures (e.g., blood vessels) because the targeted signal (having a frequency tuned to a target neural structure) will not have the same modulating effects on the non-target structures.

Accordingly, passive bioelectric properties, such as complex impedance and resistance, can be used by the system 100 before, during, and/or after neuromodulation therapy to guide one or more treatment parameters. For example, before, during, and/or after treatment, impedance or resistance measurements may be used to confirm and/or detect contact between one or more electrodes 244 and the adjacent tissue. The impedance or resistance measurements can also be used to detect whether the electrodes 244 are placed appropriately with respect to the targeted tissue type by determining whether the recorded spectra have a shape consistent with the expected tissue types and/or whether serially collected spectra were reproducible. In some embodiments, impedance or resistance measurements may be used to identify a boundary for the treatment zone (e.g., specific neural tissue that are to be disrupted), anatomical landmarks, anatomical structures to avoid (e.g., vascular structures or neural tissue that should not be disrupted), and other aspects of delivering energy to tissue.

The bioelectric information can be used to produce a spectral profile or map of the different anatomical features tissues at the target site, and the anatomical mapping can be visualized in a 3D or 2D image via the display 112 and/or other user interface to guide the selection of a suitable treatment site. This neural and anatomical mapping allows the system 100 to accurately detect and therapeutically modulate neural fibers associated with certain neurological conditions or disorders to be treated. In addition, anatomical mapping also allows the clinician to identify certain structures that the clinician may wish to avoid during therapeutic neural modulation (e.g., certain arteries). The neural and anatomical bioelectric properties detected by the system 100 can also be used during and after treatment to determine the real-time effect of the therapeutic neuromodulation on the treatment site. For example, the evaluation/feedback algorithms 110 can also compare the detected neural locations and/or activity before and after therapeutic neuromodulation, and compare the change in neural activity to a predetermined threshold to assess whether the application of therapeutic neuromodulation was effective across the treatment site.

FIG. 14B is a block diagram illustrating communication of sensor data from the handheld device 102 to the controller and subsequent determination, via the controller, of a treatment pattern for controlling delivery of energy at a specific level for a specific period of time to the tissue of interest (i.e., the targeted tissue) sufficient to ensure successful ablation/modulation of the targeted tissue while minimizing and/or preventing collateral damage to surrounding or adjacent non-targeted tissue at the target site. As shown, the end effector 214 communicates the tissue data (i.e., bioelectric properties of tissue at the target site) to the console 104. The bioelectric properties may include, but are not limited to, complex impedance, resistance, reactance, capacitance, inductance, permittivity, conductivity, dielectric properties, muscle or nerve firing voltage, muscle or nerve firing current, depolarization, hyperpolarization, magnetic field, induced electromotive force, and combinations thereof. The dielectric properties may include, for example, at least a complex relative dielectric permittivity.

In turn, console 104 (via the controller 107, monitoring system 108, and evaluation/feedback algorithms 110) is configured to process such data and determine a type of tissue at the target site. The console 104 (via the controller 107, monitoring system 108, and evaluation/feedback algorithms 110) is further configured to determine a treatment pattern to be delivered by one or more of the plurality of electrodes of the end effector based, at least in part, on identified tissues. The treatment pattern may be stored within the treatment database 504. The treatment pattern (also referred to herein as “ablation pattern”), may include various parameters associated with the delivery of energy, including, for example, a predetermined treatment time, a precise level of energy to be delivered, and a predetermined impedance threshold for that particular tissue. The console 104 (via the controller 107, monitoring system 108, and evaluation/feedback algorithms 110) is configured to tune energy output (i.e., delivery of electrical therapeutic stimulation) based on the treatment pattern of a tissue of interest such that the energy delivered via specific leaflet pairs at a specific frequency for a predetermined period of time and up to a predetermined impedance threshold, such that energy delivery is targeted the tissue of interest while avoiding the non-targeted tissue.

The tissue database may contain a plurality of profiles of identified and known tissue types, wherein each profile may include electric signature data for the associated tissue type. The electric signature data may generally include previously identified bioelectric properties of the tissue type, including impedance profiles with known impedance threshold values associated with successful and unsuccessful ablation and/or modulation treatment of that particular tissue. Accordingly, the console 104 (via the controller 107, monitoring system 108, and evaluation/feedback algorithms 110) is configured to process data received from the end effector 214 (i.e., bioelectric properties of one or more tissues at the target site) and determine a type of tissue at the target site, and a treatment pattern for each of the one or more identified tissue types based on a comparison of the data with the electric signature data stored in each of the profiles of the tissue database 502. Upon a positive correlation between data sets, the console 104 is configured to identify a matching profile and thus determine the one or more tissue types at the target site and the respective treatment patterns of each.

FIG. 14C is a block diagram illustrating delivery of energy to the target site based on the treatment pattern output from the controller, monitoring of real-time feedback data associated with the targeted tissue undergoing treatment, and subsequent control over the delivery of energy based on the processing of the feedback data. Upon delivery energy from the electrodes to the targeted tissue (based on the treatment pattern), the device 102, via the electrodes/sensors, is further configured to provide the console 104 with sensed data in the form of feedback data, in real-, or near-real, time. The real-time feedback data is associated with the effect of the therapeutic stimulation on the targeted tissue. For example, feedback data may be associated with efficacy of ablation upon targeted tissue (e.g., neural tissue) during and/or after delivery of initial energy from one or more of the plurality of electrodes. The console 104 (via the controller 107, monitoring system 108, and evaluation/feedback algorithms 110) is configured to process such real-time feedback data to determine if certain properties of the targeted tissue undergoing treatment (e.g., tissue temperature, tissue impedance, etc.) reach predetermined thresholds for irreversible tissue damage.

More specifically, the console 104 (via the controller 107, monitoring system 108, and evaluation/feedback algorithms 110) is configured to automatically control delivery of energy to the targeted tissue based on the processing of the real-time feedback data, wherein such data includes at least impedance measurement data associated with the targeted tissue collected during delivery of energy to the targeted tissue. The console 104 (via the controller 107, monitoring system 108, and evaluation/feedback algorithms 110) is configured to process impedance measurement data to detect a slope change event (e.g., an asymptotic rise) within an impedance profile associated with the treatment, wherein, with reference to the predetermined impedance threshold, the slope change event is indicative of whether the ablation/modulation of the targeted tissue is successful. In turn, the controller 107 can automatically tune energy output individually for the one or more electrodes after an initial level of energy has been delivered based, at least in part, on monitoring and processing of the real-time feedback data, most notably impedance data, to ensure the desired ablation/modulation is achieved. For example, once a slope change event (e.g., an asymptotic rise) within an impedance profile is detected, with reference to the predetermined impedance threshold of the targeted tissue (which is known via the treatment pattern), the application of therapeutic energy can be terminated to allow the tissue to remain intact and to further prevent and/or minimize collateral damage to surrounding or adjacent non-targeted tissue. For example, in certain embodiments, the energy delivery can automatically be tuned based on an evaluation/feedback algorithm (e.g., the evaluation/feedback algorithm 110 of FIG. 1A) stored on the console 104.

For example, in one embodiment, the console 104 (via the controller 107, monitoring system 108, and evaluation/feedback algorithms 110) is configured to process the impedance measurement data (received as part of the real-time feedback data) to calculate at least one of a baseline impedance value prior to delivery of energy from electrodes to the tissue for the determination of whether a given leaflet pair is available and an active impedance value during delivery of energy from electrodes of an available leaflet pair to the tissue.

As previously described herein with respect to the availability assessment, the console unit is configured to perform a secondary baseline impedance check on any active leaflet pairs during therapy. The console unit determines the availability of each of leaflet pair of a given set based on a comparison of the calculated baseline impedance value with a predetermined range of baseline impedance values. Again, a pair of the support structures is determined to be available for applying treatment when the calculated baseline value falls within the predetermined range of baseline impedance values and determined to be unavailable for applying treatment when the calculated baseline value falls outside the predetermined range of baseline impedance values. Once a secondary baseline impedance check has been performed, the console unit is further configured to process additional feedback data, wherein such additional data is in the form of an elapsed time of delivery of energy from electrodes of an available leaflet pair to the tissue. The console unit is configured to compare the elapsed time with the predetermined leaflet pair treatment time to determine a status of a given leaflet pair. The predetermined leaflet pair treatment time is generally calculated based on a predetermined therapy duration (governed by a particular treatment pattern for the given procedure), wherein the therapy duration is divided by the number of available leaflet pairs. In the event that the elapsed time of delivery of energy for a given leaflet pair exceeds the predetermined treatment time, then the console unit further makes a determination as to whether all available leaflet pairs of a given set have delivered treatment (since that last instance in which the treatment time has been calculated). In the event that not all of the available leaflet pairs of a given set have delivered treatment, the console unit further cycles through remaining available leaflet pairs of a given set and delivers energy therefrom, in a manner previously described. In the event that all available leaflet pairs of a given set have delivered treatment (since that last instance in which the treatment time has been calculated), the console unit further makes a determination as to whether there are any incomplete leaflet pairs of the given set left (i.e., any leaflet pairs still in-progress and receiving, or waiting to receive, energy to be delivered to the targeted tissue). In the event that there are no incomplete leaflet pairs of the given set present, then the console unit determines a specific treatment to be successful and thereby stops transmission of energy to the target site and outputs, via the interface 112, an alert (audible and/or visual) to the user indicating that that the specific treatment is complete and provides selectable post-procedure options from which the user may select (i.e., perform additional treatments, treat other side of a given nasal cavity, treat the other nasal cavity, etc.).

In the event that there are some incomplete leaflet pairs remaining, the console unit further makes a determination as to whether the total elapsed time is greater than or equal to the therapy duration by no greater than 10 seconds. If it is determined that the total elapsed time is not greater than or equal to the therapy duration by no greater than 10 seconds, then the console unit is configured to recalculate the treatment time (i.e., therapy duration is divided by the number of available leaflet pairs) and continue to cycle through the incomplete leaflet pairs and delivery energy thereto. If it is determined that the total elapsed time is greater than or equal to the therapy duration by no greater than 10 seconds, then the console unit further makes a determination as to whether the total elapsed time is greater than or equal to the therapy duration by no greater than 3 seconds. If it is determined that the total elapsed time is not greater than or equal to the therapy duration by no greater than 3 seconds, then the console unit is configured to set the treatment time as the time remaining for treatment and continue to cycle through available and incomplete leaflet pairs and proceed to process the active impedance value of such available and incomplete leaflet pairs, as will be described in greater detail herein. If it is determined that the total elapsed time is greater than or equal to the therapy duration by no greater than 3 seconds, then the console unit is configured to make a determination as to whether currently available leaflet pairs are incomplete. If it is determined that currently available leaflet pairs are in the incomplete status, then the console unit is configured to continue delivering energy to the available leaflet pairs and set the treatment time as the time remaining for treatment and continue to cycle through the available and incomplete leaflet pairs and proceed to process the active impedance value of such available and incomplete leaflet pairs, as will be described in greater detail herein. If it is determined that currently available leaflet pairs are not in the incomplete status, then the console unit determines such leaflet pairs to be in an unsuccessful state and thereby stops transmission of energy to the target site and outputs, via the interface 112, an alert (audible and/or visual, such as the blue color coding of the given leaflet pair(s) indicating the unsuccessful status) to the user indicating that that the specific treatment is finished and provides selectable post-procedure options from which the user may select (i.e., perform additional treatments, treat other side of a given nasal cavity, treat the other nasal cavity, etc.). In the event that the elapsed time of delivery of energy for a given leaflet pair does not exceed the predetermined treatment time, then the console unit is configured to process the active impedance value to determine efficacy of ablation/modulation of the targeted tissue (i.e., a determination as to whether a leaflet pair is in a successful state or an unsuccessful state). In particular, the console 104 (via the controller 107, monitoring system 108, and evaluation/feedback algorithms 110) may be configured to process the active impedance value using an algorithm to determine efficacy of ablation/modulation of the targeted tissue based on a comparison of the active impedance value with at least one of a predetermined minimum impedance value, a predetermined low terminal impedance value, and a predetermined high terminal impedance value. For example, the impedance values (i.e., predetermined minimum impedance value, predetermined low terminal impedance value, and predetermined high terminal impedance value) may range between approximately 40 ohms and 2 kohms. In particular, the predetermined minimum impedance value may be approximately 40 ohms, the predetermined low terminal impedance value may be approximately 800 ohms, and the predetermined high terminal impedance value may be approximately 2 kohms.

In the event that the active impedance value is less than the predetermined minimum impedance value, the console 104 is configured to determine that ablation/modulation is unsuccessful and then further disables energy delivery from the one or more electrodes of the leaflet pair, and further outputs, via the interface 112, an alert (audible and/or visual, such as the blue color coding of the given leaflet pair(s) indicating the unsuccessful status) to the user. The console unit then makes as determination as to whether other leaflet pairs in the given set is complete (i.e., whether such leaflet pair has a status of successful or unsuccessful). In the event that it is determined that other leaflet pairs are incomplete (i.e., have not yet reached either a successful or unsuccessful status), then the console unit further cycles through such remaining available leaflet pairs of the given set and delivers energy therefrom, in a manner previously described. In the event that is determined that other leaflet pairs in the given are in fact already complete, then the console unit further makes a determination as to whether all available leaflet pairs of a given set have delivered treatment (since that last instance in which the treatment time has been calculated), as previously described herein.

In the event that the active impedance value is not less than the predetermined minimum impedance value, then the console unit makes a determination as to whether the active impedance value is greater than the predetermined low terminal impedance value. If the active impedance value is less than the predetermined low terminal impedance value, then the console unit is configured to continue to cycle through available leaflet pairs of the given set and deliver energy therefrom, in a manner previously described. In the event that the active impedance value is greater than the predetermined low terminal impedance value, then the console unit is configured to make a determination as to whether a slope event is detected. The slope event is an assessment to determine whether there is an upward slope of impedance to exceed a specified threshold. In particular, the console unit 104 is configured to calculate a slope change for the detection of a slope event. In the absence of detecting a slope event, the console unit is further configured to make a determination as to whether the active impedance value is greater than the predetermined high terminal impedance value. If the active impedance value is not greater than the predetermined high terminal impedance value, then the console unit is configured to continue to cycle through available leaflet pairs of the given set and deliver energy therefrom, in a manner previously described. If the active impedance value is greater than the predetermined high terminal impedance value, then the console unit determines the leaflet pair to be in an in an unsuccessful state and further disables energy delivery from the one or more electrodes of the leaflet pair, and further outputs, via the interface 112, an alert (audible and/or visual, such as the blue color coding of the given leaflet pair(s) indicating the unsuccessful status) to the user.

In the event that a slope event is detected, the console unit is configured to make a determination as to whether a negative slope event is detected. If a negative slope event is not detected, then the console unit determines the leaflet pair to be in an in an unsuccessful state and further disables energy delivery from the one or more electrodes of the leaflet pair, and further outputs, via the interface 112, an alert (audible and/or visual, such as the blue color coding of the given leaflet pair(s) indicating the unsuccessful status) to the user.

If a negative slope event is detected, the console 104 is configured to determine that leaflet pair is in a successful state and further disables energy delivery from the one or more electrodes of the leaflet pair, and further outputs, via the interface 112, an alert (audible and/or visual, such as the green color coding of the given leaflet pair(s) indicating the successful status) to the user. The console unit then makes as determination as to whether other leaflet pairs in the given set is complete (i.e., whether such leaflet pair has a status of successful or unsuccessful), in a manner previously described herein.

As previously described, the electrodes are configured to be independently controlled and activated by the controller 107 (in conjunction with the evaluation/feedback algorithms 110) to thereby deliver energy independent of one another. Accordingly, the controller 107 can tune energy output individually for the one or more electrodes after an initial level of energy has been delivered based, at least in part, on feedback data. For example, once the threshold is reached, the application of therapeutic stimulation energy can be terminated to allow the tissue to remain intact. In other embodiments, if the threshold has not been reached, the controller can maintain, reduce, or increase energy output to a given electrode until such threshold is reached. Accordingly, the energy delivery of any given electrode can automatically be tuned based on an evaluation/feedback algorithm (e.g., the evaluation/feedback algorithm 110 of FIG. 1A) stored on a console (e.g., the console 104 of FIG. 1A) operably coupled to the end effector. For example, at least some of the electrodes may have different levels of energy to be delivered at respective positions sufficient to ablate neural tissue at the respective positions based on the feedback data received for the respective locations.

In some embodiments, the condition includes a peripheral neurological condition. The peripheral neurological condition may be associated with a nasal condition or a non-nasal condition of the patient. For example, the non-nasal condition may include atrial fibrillation (AF). In some embodiments, the nasal condition may include rhinosinusitis. Accordingly, in some embodiments, the target site is within a sino-nasal cavity of the patient. The delivery of the ablation energy may result in disruption of multiple neural signals to, and/or result in local hypoxia of, mucus producing and/or mucosal engorgement elements within the sino-nasal cavity of the patient. The targeted tissue is proximate or inferior to a sphenopalatine foramen. Yet still, delivery of the ablation energy may result in therapeutic modulation of postganglionic parasympathetic nerves innervating nasal mucosa at foramina and or microforamina of a palatine bone of the patient. In particular, delivery of the ablation energy causes multiple points of interruption of neural branches extending through foramina and microforamina of palatine bone. Yet still, in some embodiments, delivery of the ablation energy may cause thrombus formation within one or more blood vessels associated with mucus producing and/or mucosal engorgement elements within the nose. The resulting local hypoxia of the mucus producing and/or mucosal engorgement elements may result in decreased mucosal engorgement to thereby increase volumetric flow through a nasal passage of the patient. Additionally, or alternatively, the resulting local hypoxia may cause neuronal degeneration.

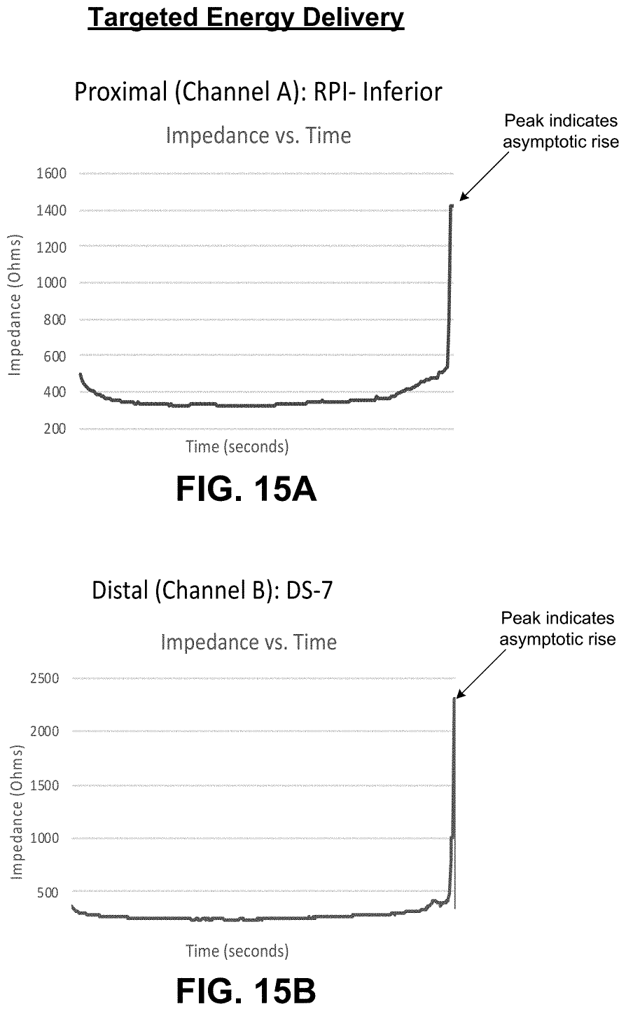

FIGS. 15A and 15B are graphs illustrating impedance profiles of two different sets of electrodes delivering energy to respective portions of targeted tissue, wherein the graphs illustrate a slope change event (e.g., asymptotic rise) which is indicative of whether the ablation/modulation of the targeted tissue is successful.

As previously described, systems and methods are further configured to receive and process real-time feedback data associated with the targeted tissue undergoing treatment to further ensure that energy delivered is maintained within the scope of the treatment pattern. More specifically, the systems and methods are configured to automatically control delivery of energy to the targeted tissue based on the processing of the real-time feedback data, wherein such data includes at least impedance measurement data associated with the targeted tissue collected during delivery of energy to the targeted tissue. The controller is configured to process impedance measurement data to detect a slope change event (e.g., an asymptotic rise) within an impedance profile associated with the treatment, wherein, with reference to the predetermined impedance threshold, the slope change event is indicative of whether the ablation/modulation of the targeted tissue is successful. In turn, the controller is configured to automatically control the delivery of energy to the targeted tissue based on real-time monitoring of feedback data, most notably impedance data, to ensure the desired ablation/modulation is achieved.

As a result, the systems and methods are able to ensure that optimal energy is delivered in order to delay the onset of impedance roll-off, until the target ablation/modulation depth is achieved, while maintaining clinically relevant treatment time. Accordingly, the invention solves the problem of causing unnecessary collateral damage to non-targeted tissue during a procedure involving the application of electrotherapeutic stimulation at a target site composed of a variety of tissue types.

Following the delivery of energy from one or more electrodes of leaflet pairs, resulting in either successful or unsuccessful treatment of respective targeted tissue, the console unit 104 performs post-treatment analysis. The post-treatment analysis includes a determination of any prior treatments performed, including prior use of the electrodes on prior targeted tissue for a given nasal cavity, a status of such prior use, including whether such treatment was successful or unsuccessful, and a determination of any and all further treatments to be performed. In turn, the console unit provides, via the interactive interface, one or more post-procedure inputs from which the user may select for controlling subsequent use of the treatment device to ensure that the overall procedure (i.e., treatment of rhinosinusitis) is completed by ensuring that all portions of targeted tissue undergo treatment.

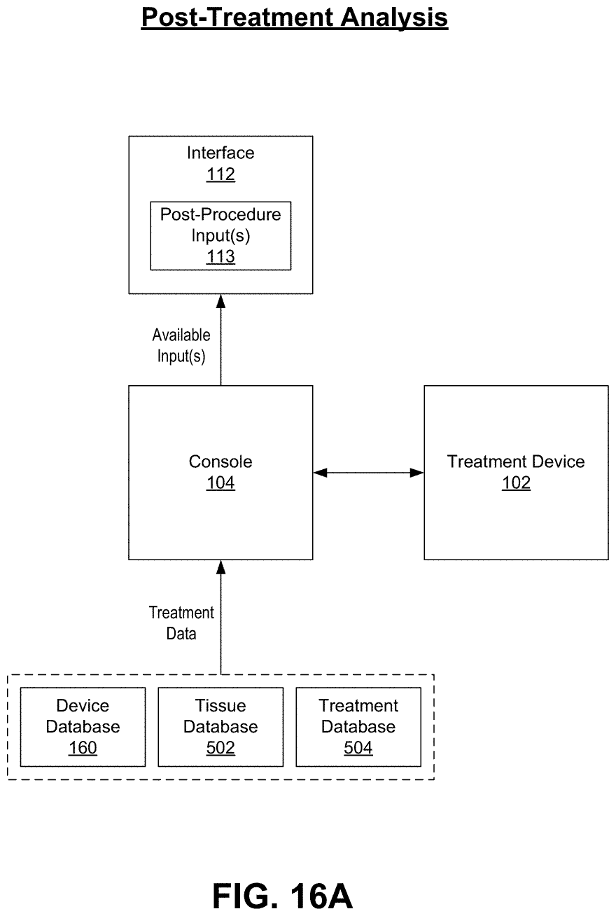

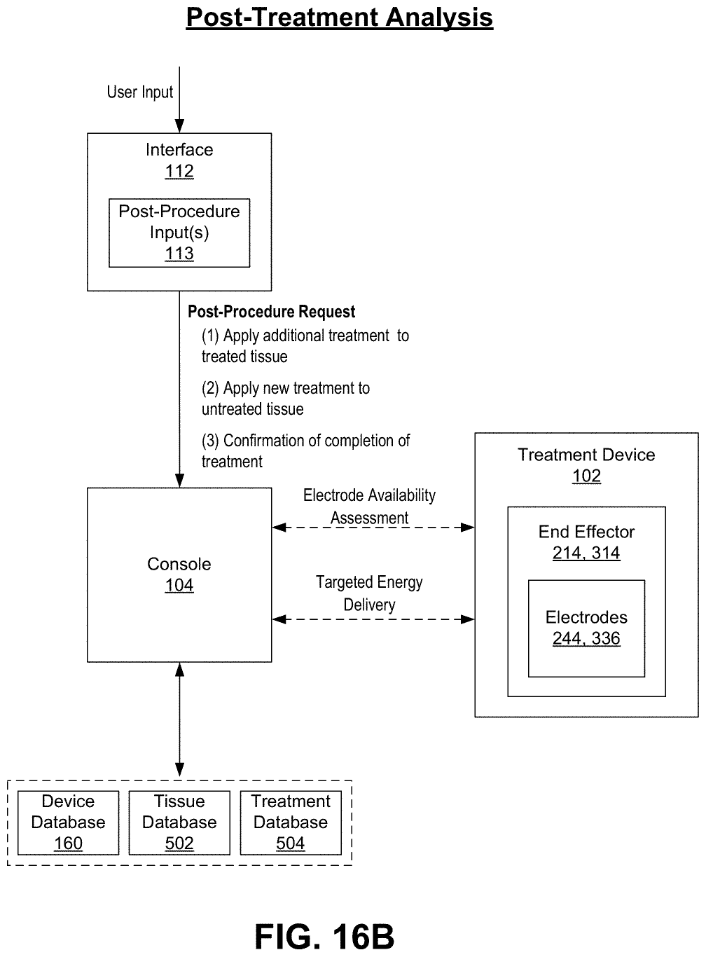

FIGS. 16A and 16B are block diagrams illustrating post-treatment analysis, including post-procedure inputs provided by the console unit 104 from which a user may select for controlling subsequent use of the treatment device 102 to ensure that the overall procedure is completed. As shown, the console unit is operably associated with the treatment device 102 and provides various post-procedure inputs 113 to a user, via the interface 112, from which a user may select depending on what treatments have been previously performed. In particular, the console unit 104 is configured to determine, in part, which particular post-procedure options are available for selection based, at least in part, on treatment data of the given device 102, which includes data from at least one of the device database 160, tissue database 502, and treatment database 504. In other words, a given device may have a profile stored within the device database 160, wherein the device profile may generally include a history of prior use, including, for example prior use of one or more electrodes, and the associated leaflet pairs, in delivering energy to one or more associated target sites within either one of the left and rights sides of the sino-nasal cavity and an indication of whether treatment applied is complete for either of the left and right sides of the sino-nasal cavity.

Accordingly, the one or more post-procedure inputs may include, for example, an option for initiating one or more additional applications of treatment to a selected one of the left and right sides of the sino-nasal cavity having already undergone treatment, an option for initiating application of treatment to an untreated one of the left and right sides of the sino-nasal cavity, or an option simply confirming completion of entire procedure.

Upon the discontinuing of RF therapy from the targeted energy delivery portion of the procedure, as previously described with reference to FIGS. 14A-14C, the console unit may be configured to make a determination as to whether the other nasal cavity has been treated, which will determine the specific post-procedure options that will be provided to the user. For example, in the event that the other nasal cavity has not yet been treated, the console unit is configured to provide the user with a set of options for the first cavity that has just undergone therapy. In this instance, a user may be presenting with at least three different options, including the option of initiating one or more additional applications of treatment to the first nasal cavity having already undergone treatment, the option of initiating application of treatment to the second nasal cavity that has yet to undergo any treatment, and the option of confirming completion of entire procedure.

In the event that the user selects for additional applications of treatment to be applied to the first cavity having already undergone treatment, the console unit is configured to return to and initiate an impedance assessment of certain leaflet pairs and the associated electrodes. In particular, the console unit is able to determine which leaflet pairs have already delivered treatment in a successful manner (i.e., have a successful status) based on treatment data for any given leaflet pair. Thus, the availability assessment is only performed on those leaflet pairs that were not deemed to be in a successful state. Depending on the availability of one or more electrodes of leaflet pairs, the console unit then presents the user with operational inputs, including the option of initiating treatment, including the targeted energy delivery process, as previously described herein.

In the event that the user selects for the other nasal cavity to be treated, the console unit is configured to return to and initiate an impedance assessment of all leaflet pairs and the associated electrodes. In particular, the console unit is able to determine that the other nasal cavity has not yet undergone treatment and thus all data associated with all leaflet pairs is cleared (as opposed to prior treatment data of leaflet pairs associated with treatment of the first nasal cavity). The console unit then presents the user with operational inputs, including the option of initiating treatment, including the targeted energy delivery process, as previously described herein.

In the event that the user selects and confirms that the procedure is entirely complete, then the console unit is configured to set the system back to the initial setup state and further output, via the interface, an audible and/or visual alert that the procedure is complete (i.e., text indicating the procedure is complete and further advising the user to disconnect the device). In the event that the other nasal cavity has already been treated (i.e., the first nasal cavity has been treated and the second nasal cavity just underwent treatment), the console unit is configured to provide the user with a smaller set of options. In this instance, a user may be presenting with at least two different options, including the option of initiating one or more additional applications of treatment to the second nasal cavity having just undergone treatment and the option of confirming completion of entire procedure.

Accordingly, the systems and methods of the present invention provide an intuitive, user-friendly, and semi-automated means of treating rhinosinusitis conditions, including precise and focused application of energy to the intended targeted tissue without causing collateral and unintended damage or disruption to other tissue and/or structures. Thus, the efficacy of a vidian neurectomy procedure can be achieved with the systems and methods of the present invention without the drawbacks discussed above. Most notably, the console unit provides a user (i.e., surgeon or other medical professional) with relatively simple operational instructions, in the form of step-by-step guidance via an interactive interface, for performing the procedure, such as directing the user to select a specific nasal cavity to treat, providing indications (both visual and audible) as to when the treatment device is ready to perform a given treatment, providing automated control over the delivery of energy to the targeted tissue upon user-selected input to initiate treatment, and further providing a status of therapy during the procedure and after the procedure, including indications (e.g., visual and/or audible) as to whether the treatment is successful or unsuccessful. Accordingly, such treatment is effective at treating rhinosinusitis conditions while greatly reducing the risk of causing lateral damage or disruption to other tissue or structures (i.e., non-targeted tissue, such as blood vessels, bone, and non-targeted neural tissue), thereby reducing the likelihood of unintended complications and side effects.

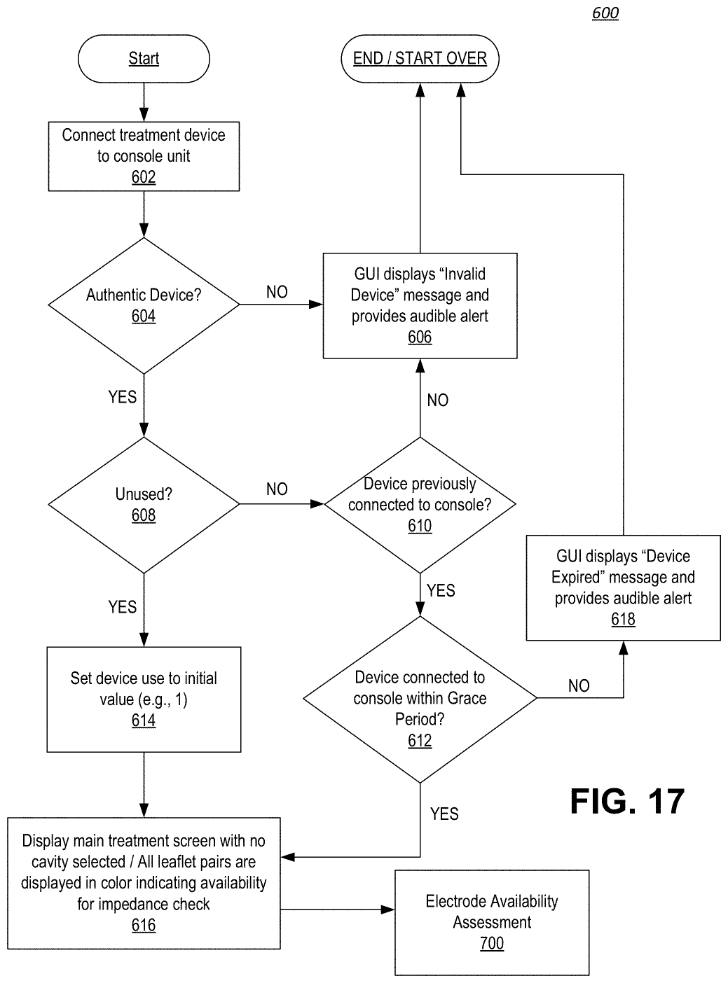

FIG. 17 is a flow diagram illustrating one embodiment of a method 600 for authenticating a handheld treatment device to be used with the console unit of the present disclosure. The method 600 includes connecting a treatment device to the console unit (operation 602). A determination is then made in operation 604 as to whether the device is authentic. At this point, the identifying data associated with the treatment device is analyzed upon connection of the treatment device to the console unit and authenticity is determined based on the analysis of the identifying data. If it is determined in operation 604 that the device is not authentic, an alert is provided (via a GUI) indicating to the user that device is inauthentic/invalid/incompatible, the alert including at least one of an audible (specific audible tone) and visual alert (specific text providing a message and further suggested action) (operation 606). In turn, the authentication process ends and will resume upon connection of another device to the console unit.

If it is determined in operation 604 that the device is authentic, then a determination is made in operation 608 as to whether the device is unused. If it is determined in operation 608 that the device has been previously used, then a determination is made in operation 610 as to whether the device was previously connected to the console unit. If it is determined in operation 610 that the device was not previously connected to the console unit, then an alert is provided (via a GUI) indicating to the user that device is inauthentic/invalid/incompatible, the alert including at least one of an audible (specific audible tone) and visual alert (specific text providing a message and further suggested action) (operation 606). In turn, the authentication process ends and will resume upon connection of another device to the console unit. If it is determined in operation 610 that the device was previously connected to the console unit, then a determination is made in operation 612 as to whether the device was connected to the console unit within a predetermined grace period (a period of elapsed time since first connection with console unit, such as 90 minutes). If it is determined in operation 612 that the device was not previously connected to the console unit within the predetermined grace period, then an alert is provided (via a GUI) indicating to the user that device is inauthentic/invalid/incompatible, the alert including at least one of an audible (specific audible tone) and visual alert (specific text providing a message that the device has expired and further suggesting additional actions) (operation 618). If it is determined in operation 612 that the device was previously connected to the console unit within the predetermined grace period, then a main treatment screen (i.e., a home screen or the like) is displayed to the user via the GUI, in which neither nasal cavity is selected and all leaflet pairs are displayed in a color (i.e., gray color) indicating availability to undergo and initial availability assessment (i.e., baseline impedance check) (operation 616). Similarly, if it is determined in operation 608 that the device has not been previously used, then the device use is set to an initial, baseline value (e.g., 1) (operation 614) and then the main treatment screen is displayed (operation 616). At this point, the electrode availability assessment (method 700) is available.

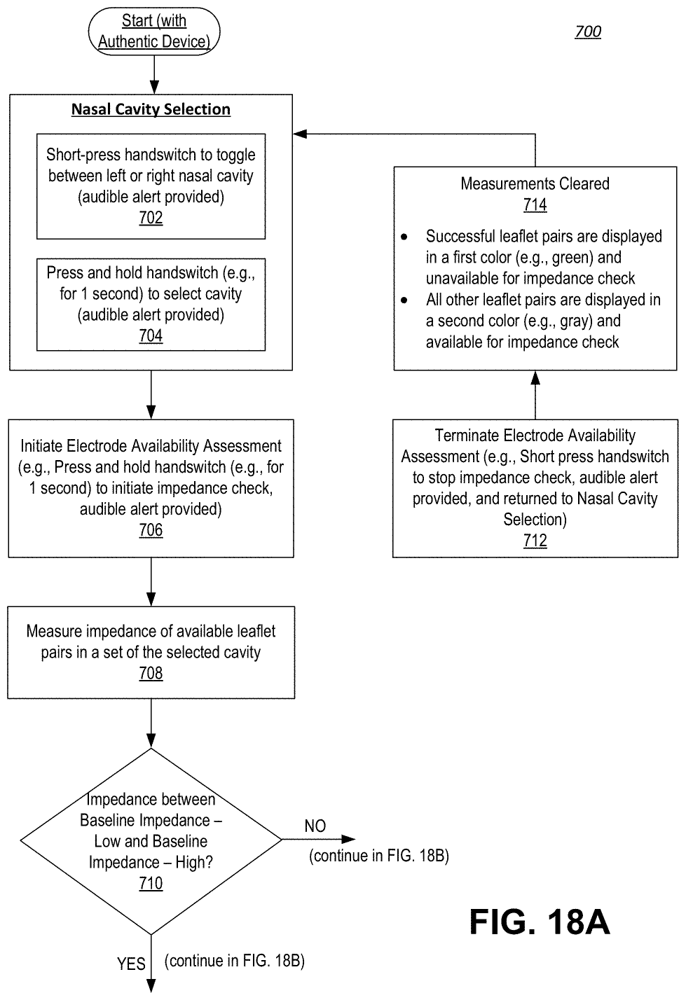

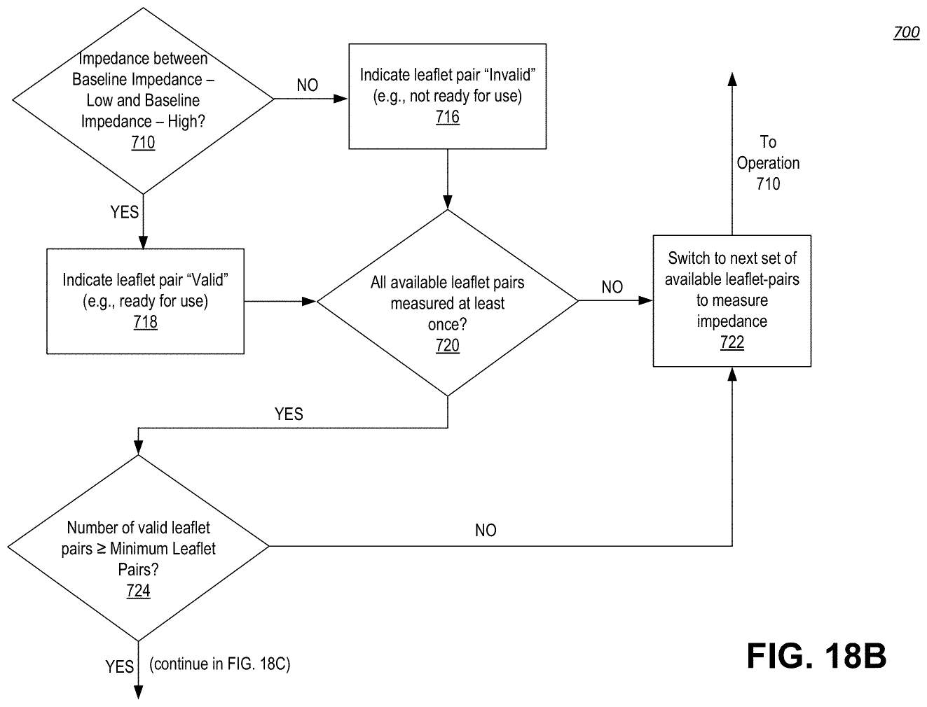

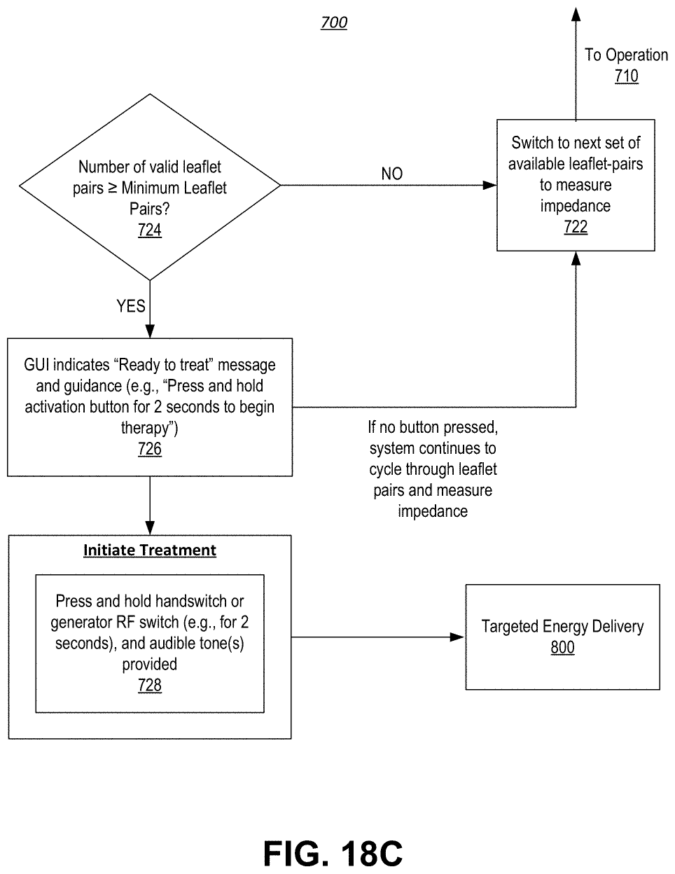

FIGS. 18A-18C show a continuous flow diagram illustrating a method 700 for providing an availability assessment of one or more electrodes of an end effector of a handheld device and subsequently providing an indication (i.e., visual and/or audible alert(s)) as to whether the device is primed and ready to perform treatment in the selected location.

Upon authenticating the device, a user is presented with a main screen, in which they may select a specific nasal cavity in which treatment should be applied (in the event that the procedure involves treatment of a nasal condition, such as rhinosinusitis). A user is presented with the option to select either the right nasal cavity or the left nasal cavity. A user need only short-press a handswitch button (provided on the handheld treatment device) to toggle between the left or right nasal cavity (operation 702), wherein an audible tone may further be provided indicating toggling between selections. The user then need only press and hold the handswitch for a period of time (e.g., 1 second) to select the right or left cavity option (operation 704), wherein an audible tone may further be provided confirming the selection.

Upon confirming a selection, the user may then be presented with an option of initiating an electrode availability assessment (i.e., baseline impedance check), and, upon pressing and holding the handswitch for a period of time (e.g., 1 second) the impedance check of one or more leaflet pairs in a given set of a selected nasal cavity may be initiated (operation 706). In particular, the impedance of electrodes associated with leaflet pairs in a given set of a selected nasal cavity begins (operation 708).

For example, with respect to the multi-segmented end effector 314, the right nasal cavity may be associated with three different sets of leaflet pairs and the left nasal cavity may be associated with another three different sets of leaflet pairs. In particular, a first set of leaflet pairs associated with the right nasal cavity may include one or more leaflet pairs of a first portion of the distal stage and one or more leaflet pairs associated with an outer right and superior portion of the proximal stage. A second set of leaflet pairs associated with the right nasal cavity may include one or more leaflet pairs of a second portion of the distal stage and one or more leaflet pairs of a left inner and inferior portion of the proximal stage. A third set of leaflet pairs associated with the right nasal cavity may include one or more leaflet pairs of a third portion of the distal stage and one or more leaflet pairs of a right inner and superior portion of the proximal stage.

Similarly, due to the bilateral geometry of the end effector, the sets of leaflet pairs associated with the left nasal cavity may generally mirror the sets of leaflet pairs associated with the right nasal cavity. In particular, the first set of leaflet pairs associated with the left nasal cavity include one or more leaflet pairs of a fourth portion of the distal stage and one or more leaflet pairs associated with an outer left right and superior portion of the proximal stage. A second set of leaflet pairs associated with the left nasal cavity may include one or more leaflet pairs of a fifth portion of the distal stage and one or more leaflet pairs of a right inner and inferior portion of the proximal stage. A third set of leaflet pairs associated with the left nasal cavity may include one or more leaflet pairs of a sixth portion of the distal stage and one or more leaflet pairs of a left inner and superior portion of the proximal stage.

A determination is made in operation 710 as to whether a calculated baseline impedance of a given leaflet pair is within a range of baseline impedance values, specifically between a baseline impedance—low value and a baseline impedance—high value. The baseline impedance low may have a value of approximately 100 ohms and the baseline impedance—high may have a value of approximately 1 kohms. Depending on the determination made in operation 710, the given leaflet pair may be determined to be available or unavailable, as will be described in greater detail herein.

It should be noted that, at any point, a user may simply terminate the electrode availability assessment by simply short pressing handswitch to stop the impedance check and return to the nasal cavity selection option (operation 712), wherein an audible tone may further be provided confirming the selection to stop the impedance check. Upon terminating the availability assessment, impedance measurements are cleared, wherein successful leaflet pairs are displayed in a first color (e.g., green) and are unable to undergo an impedance check (as they are unavailable) and all other leaflet pairs are displayed to the user in a second color (e.g., gray) and able to undergo an availability assessment (operation 714).

If it is determined in operation 710 that a calculated baseline impedance value of a given leaflet pair does not fall within the predetermined range of baseline impedance values (low and high), then an alert is provided (via a GUI) indicating to the user that the leaflet pair is invalid and thus not ready for use, the alert including at least one of an audible (specific audible tone) and visual alert (specific text providing a message that the leaflet pair is not ready and a color coding, such as gray, indicating the unavailability of the leaflet pair) (operation 716). If it is determined in operation 710 that a calculated baseline impedance value of a given leaflet pair falls within the predetermined range of baseline impedance values (low and high), then an alert is provided (via a GUI) indicating to the user that the leaflet pair is valid and thus ready for use, the alert including at least one of an audible (specific audible tone) and visual alert (specific text providing a message that the leaflet pair is ready and a color coding, such as blue, indicating the unavailability of the leaflet pair) (operation 718).

After operations 716 and 718, a determination is then made in operation 720 as to whether all available leaflet pairs have been measured (i.e., undergone impedance check) at least once. If it is determined in operation 720 that there some available leaflet pairs that have not been measured at least once, then the next set of available leaflet pairs undergo impedance measurements (operation 722) and then continue to operation 710. If it is determined in operation 720 that all available leaflet pairs have in fact been measured at least once, then a determination is made in operation 724 as to whether the number of valid leaflet pairs is greater than or equal to a predetermined minimum number of leaflet pairs (e.g., minimum of 1 leaflet pair). If it is determined in operation 724 that the number of valid leaflet pairs is not greater than or equal to then predetermined minimum number of leaflet pairs, then the next set of available leaflet pairs undergo impedance measurements (operation 722) and then continue to operation 710. If it is determined in operation 724 that the number of valid leaflet pairs is greater than or equal to then predetermined minimum number of leaflet pairs, then an alert is provided (via a GUI) indicating to the user that the device is ready to provide treatment, the alert including at least one of an audible (specific audible tone) and visual alert (specific text providing a message that the device is ready for providing treatment, as well as additional guidance to the user as how to interact with device inputs to initiate therapy (operation 726). In the event that the user presses and holds the handswitch or generator RF switch (e.g., for 2 seconds) (operation 728), then targeted energy delivery (method 800) can begin. In the event that no input is provided by the user (i.e., no activation button is pressed), the system continues to cycle through leaflet pairs and measure impedance (via operation 722).

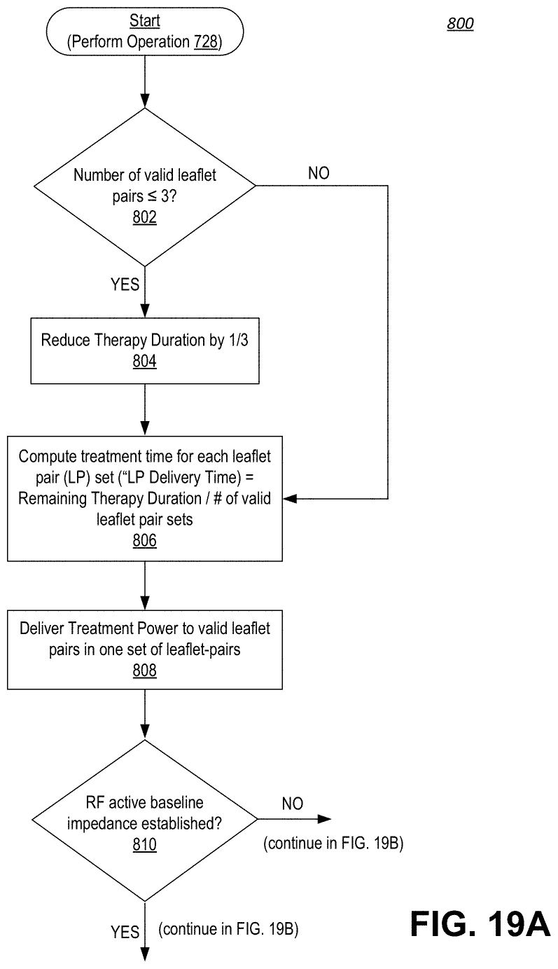

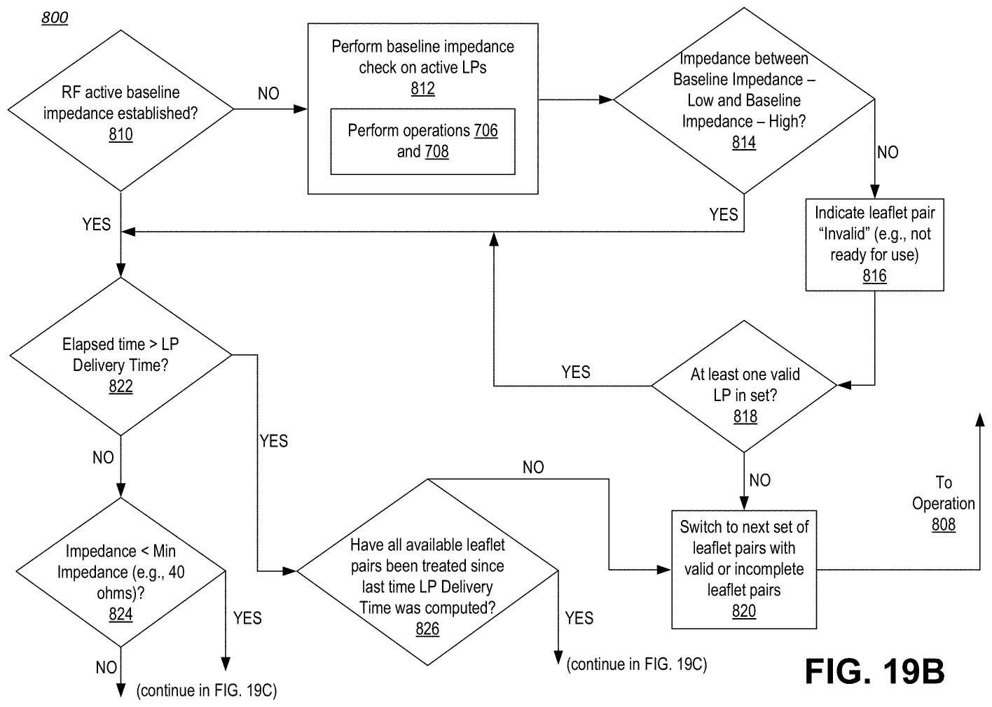

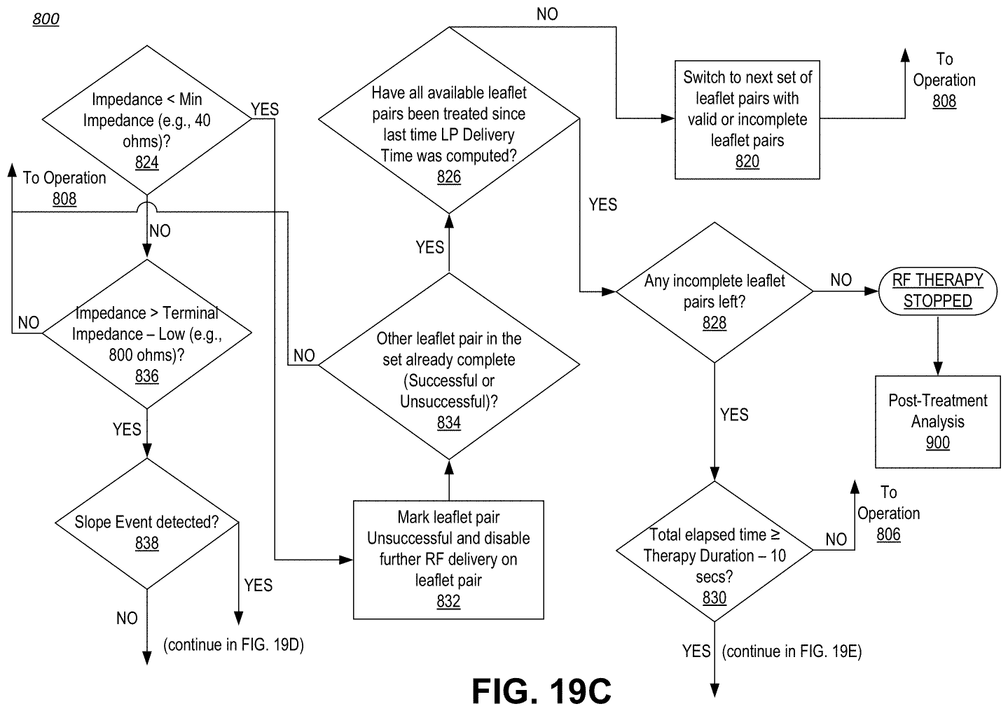

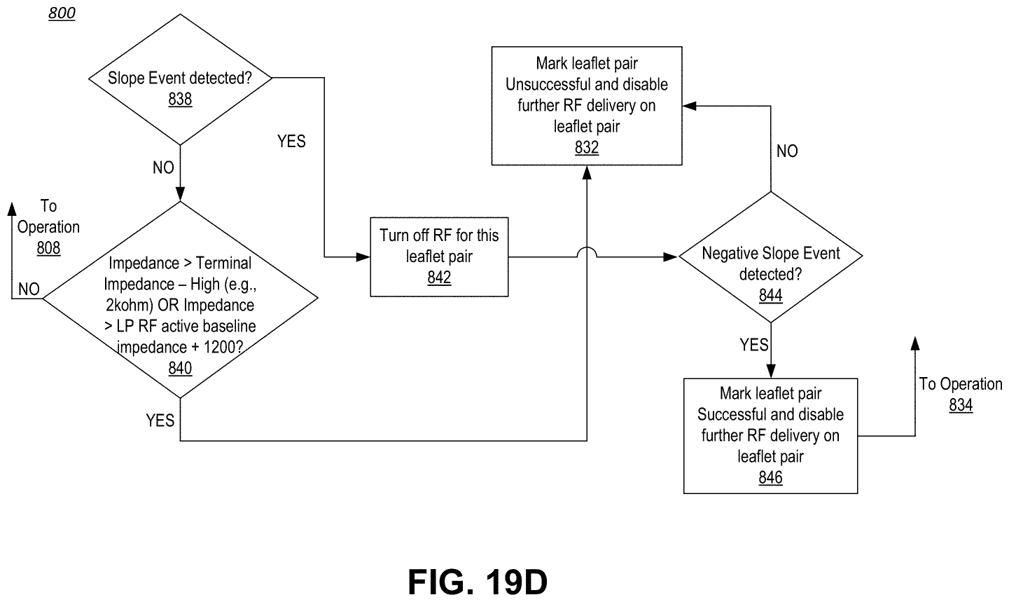

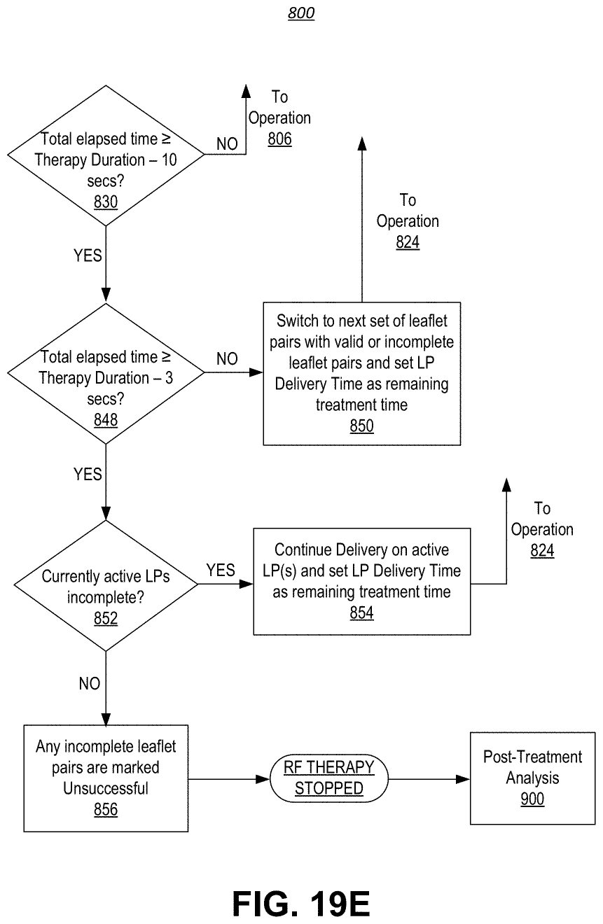

FIGS. 19A-19E show a continuous flow diagram illustrating a method 800 for targeted energy delivery to a targeted tissue based, at least in part, on a treatment pattern output from the controller, monitoring of real-time feedback data associated with the targeted tissue undergoing treatment, and subsequent control over the delivery of energy based on the processing of the feedback data. Depending on the availability of one or more electrodes for energy delivery, the user may be presented with operational inputs, including the option of initiating treatment. For example, upon performing operation 728 (user presses and holds the handswitch or generator RF switch (e.g., for 2 seconds)), the targeted energy delivery from one or more sets leaflet pairs to corresponding target sites within the selected one of the right or left nasal cavity.

A determination is made in operation 802 as to whether the number of valid leaflet pairs is less than or equal to 3. If it is determined in operation 802 that the number of valid leaflet pairs is less than or equal to 3, then therapy duration is reduced by ⅓ (one-third) (operation 804). In turn, a treatment time for each leaflet pair (LP) set is computed (referred to as “LP Delivery Time”) by dividing the remaining therapy duration by the number of valid leaflet pair sets (operation 806). If it is determined in operation 802 that the number of valid leaflet pairs is greater than 3, then operation 806 is immediately performed (and therapy duration is not reduced). Energy (treatment power) is then delivered to valid leaflet pairs in at least one set of the leaflet pairs (Operation 808). A determination is then made in operation 810 as to whether a RF active baseline impedance has been established. The RF active baseline impedance is a secondary baseline impedance check performed during RF therapy on active leaflet pairs and such a measurement is retained for each leaflet pair. If it is determined that RF active baseline impedance has not been established, then a baseline impedance check is performed on active leaflet pairs (operation 812). In particular, operations 706 and 708 are performed.

A determination is made in operation 814 as to whether a calculated baseline impedance of a given leaflet pair is within a range of baseline impedance values, specifically between a baseline impedance—low value and a baseline impedance—high value. The baseline impedance low may have a value of approximately 100 ohms and the baseline impedance—high may have a value of approximately 1 kohms. If it is determined in operation 814 that a calculated baseline impedance value of a given leaflet pair does not fall within the predetermined range of baseline impedance values (low and high), then an alert is provided (via a GUI) indicating to the user that the leaflet pair is invalid and thus not ready for use, the alert including at least one of an audible (specific audible tone) and visual alert (specific text providing a message that the leaflet pair is not ready and a color coding, such as gray, indicating the unavailability of the leaflet pair) (operation 816). A determination is then made in operation 818 as to whether there is at least one valid leaflet pair in the given set. If it is determined in operation 818 that there is not at least one valid leaflet pair in the given set, then the system switches to the next set of leaflet pairs with valid or incomplete leaflet pairs (operation 820) and then continues back to operation 808. If it is determined in operation 810 that an active baseline impedance is established, or determined in operation 818 that there is at least one valid leaflet pair in the given set, or determined in operation 814 that a calculated baseline impedance value of a given leaflet pair falls within the predetermined range of baseline impedance values (low and high), then a determination is made in operation 822 as to whether an elapsed time of delivery of energy from electrodes of an available leaflet pair to the tissue is greater than the calculated LP Delivery Time (calculated in operation 806). If it is determined in operation 822 that the elapsed time is not greater than the LP Delivery Time, then a determination is made in operation 824 as to whether the active baseline impedance is less than a predetermined minimum impedance value (e.g., 40 ohms).

If it is determined in operation 822 that the elapsed time is greater than the LP Delivery Time, then a determination is made in operation 826 as to whether all available leaflet pairs have been used (i.e., delivered treatment) since the last time the LP Delivery Time was calculated. If it is determined in operation 826 that all available leaflet pairs have not been used since the last time the LP Delivery Time was calculated, then the system switches to the next set of leaflet pairs with valid or incomplete leaflet pairs (operation 820) and then continues back to operation 808.

Referring back to the determination in operation 824, if it is determined that the active baseline impedance is less than a predetermined minimum impedance value (e.g., 40 ohms), then an alert is provided (via a GUI) indicating to the user that the leaflet pair is unsuccessful and further RF delivery from the leaflet pair is disabled (operation 832), wherein the alert includes at least one of an audible (specific audible tone) and visual alert (specific text providing a message that the leaflet pair is unsuccessful and a color coding, such as blue, indicating that the leaflet pair is unsuccessful). A determination is then made in operation 834 as to whether the other leaflet pair in the given set is complete (i.e., whether it has been deemed successful or unsuccessful). If it is determined in operation 834 that the other leaflet pair the given set is not complete, then the method proceeds to back to operation 808. If it is determined in operation 834 that the other leaflet pair the given set is complete, then the determination in operation 826 is made. If it is determined in operation 826 that all available leaflet pairs have not been used since the last time the LP Delivery Time was calculated, then the system switches to the next set of leaflet pairs with valid or incomplete leaflet pairs (operation 820) and then continues back to operation 808. If it is determined in operation 826 that all available leaflet pairs have been used since the last time the LP Delivery Time was calculated, then a determination is made in operation 828 as to whether there are any incomplete leaflet pairs left (operation 828). If it is determined in operation 828 that there are no incomplete leaflet pairs left, then the RF therapy is stopped, and the user may be presented with an alert indicating that such therapy has stopped (i.e., audible tone or visual indication) and the system then performed post-treatment analysis of method 900. If it is determined in operation 828 that there are incomplete leaflet pairs left, then a determination is made in operation 830 as to whether the total elapsed time is greater than or equal to the therapy duration by no greater than 10 seconds.

If it is determined in operation 830 that the total elapsed time is not greater than or equal to the therapy duration by no greater than 10 seconds, then the process continues back to operation 806. If it is determined that the total elapsed time is greater than or equal to the therapy duration by no greater than 10 seconds, then a subsequent determination is made in operation 848 (see FIG. 19E), which will be described in greater detail herein.

Referring back to operation 824, if it is determined that the active baseline impedance is not less than a predetermined minimum impedance value (e.g., 40 ohms), then a determination is made in operation 836 as to whether the active baseline impedance is greater than a predetermined low terminal impedance value. If it is determined in operation 836 that the active baseline impedance is not greater than the predetermined low terminal impedance value, then the method proceeds back to operation 808. If it is determined in operation 836 that the active baseline impedance is greater than the predetermined low terminal impedance value, then a determination is made in operation 838 as to whether a slope event is detected. If it is determined in operation 838 that a slope event is detected, then RF delivery is disabled for the given leaflet pair (operation 842) and a determination is then made in operation 844 as to whether a negative slope event is detected.

If it is determined in operation 844 that a negative event slope is not detected, then an alert is provided (via a GUI) indicating to the user that the leaflet pair is unsuccessful and RF delivery from the leaflet pair is disabled (operation 832), wherein the alert includes at least one of an audible (specific audible tone) and visual alert (specific text providing a message that the leaflet pair is unsuccessful and a color coding, such as blue, indicating that the leaflet pair is unsuccessful), then the method continues on to operation 834. If it is determined in operation 844 that a negative event slope is detected, then an alert is provided (via a GUI) indicating to the user that the leaflet pair is successful and RF delivery from the leaflet pair is disabled (operation 846), wherein the alert includes at least one of an audible (specific audible tone) and visual alert (specific text providing a message that the leaflet pair is successful and a color coding, such as green, indicating that the leaflet pair is successful), then the method continues on to operation 834.

Referring back to operation 838, if a slope event is not detected, then a determination is made in operation 840 as to whether the active impedance is greater than the predetermined high terminal impedance value or greater than a sum of the active impedance value with the addition of a value of 1200. If it is determined in operation 840 that the active impedance is not greater than the predetermined high terminal impedance value and not greater than a sum of the active impedance value with the addition of a value of 1200, then the method continues back to operation 808. If it is determined in operation 840 that the active impedance is greater than the predetermined high terminal impedance value or greater than a sum of the active impedance value with the addition of a value of 1200, then the method continues to operation 832. Referring to FIG. 19E, and with reference back to operation 830, if it is determined that the total elapsed time is greater than or equal to the therapy duration by no greater than 10 seconds, then a determination is made in operation 848 as to whether the total elapsed time is greater than or equal to the therapy duration by no greater than 3 seconds. If it is determined in operation 848 that the total elapsed time is not greater than or equal to the therapy duration by no greater than 3 seconds, then the system switches to the next set of leaflet pairs with valid or incomplete leaflet pairs and sets the LP Delivery Time as the remaining treatment time (operation 850) and then continues back to operation 824.

If it is determined in operation 848 that the total elapsed time is greater than or equal to the therapy duration by no greater than 3 seconds, then a determination is made in operation 852 as to whether any currently active leaflet pairs are incomplete. If it is determined in operation 852 that there are currently active leaflet pairs that are incomplete, then the system continues delivery on the active leaflet pairs and sets the LP Delivery Time as the remaining treatment time (operation 854) and then continues back to operation 824. If it is determined in operation 852 that there are no currently active leaflet pairs that are incomplete, then any incomplete leaflet pairs are marked as unsuccessful (operation 856), such that an alert is provided (via a GUI) indicating to the user that the incomplete leaflet pair(s) is unsuccessful, and the RF therapy is stopped, and the user may be presented with an alert indicating that such therapy has stopped (i.e., audible tone or visual indication) and the system then performed post-treatment analysis of method 900.

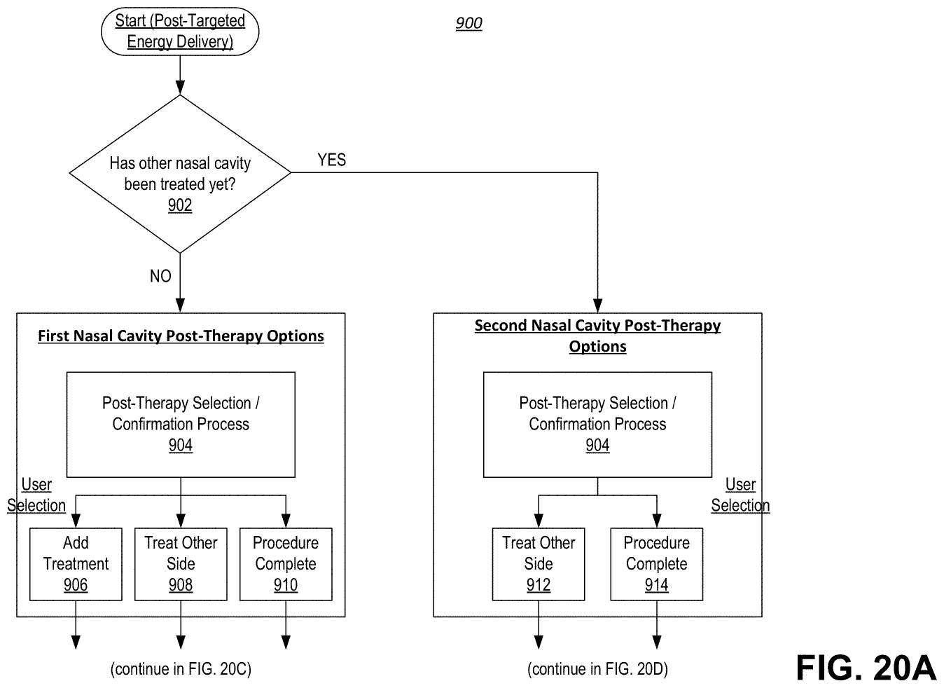

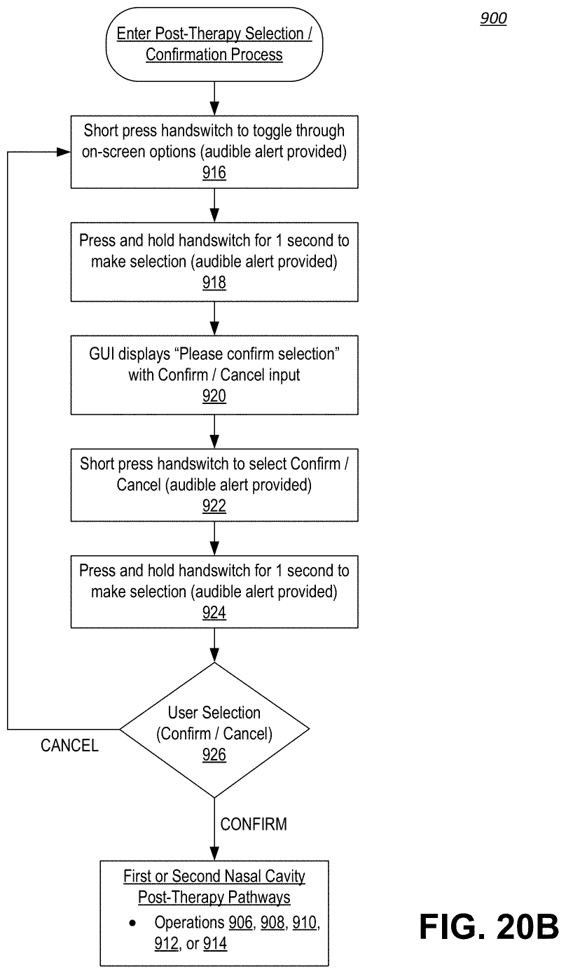

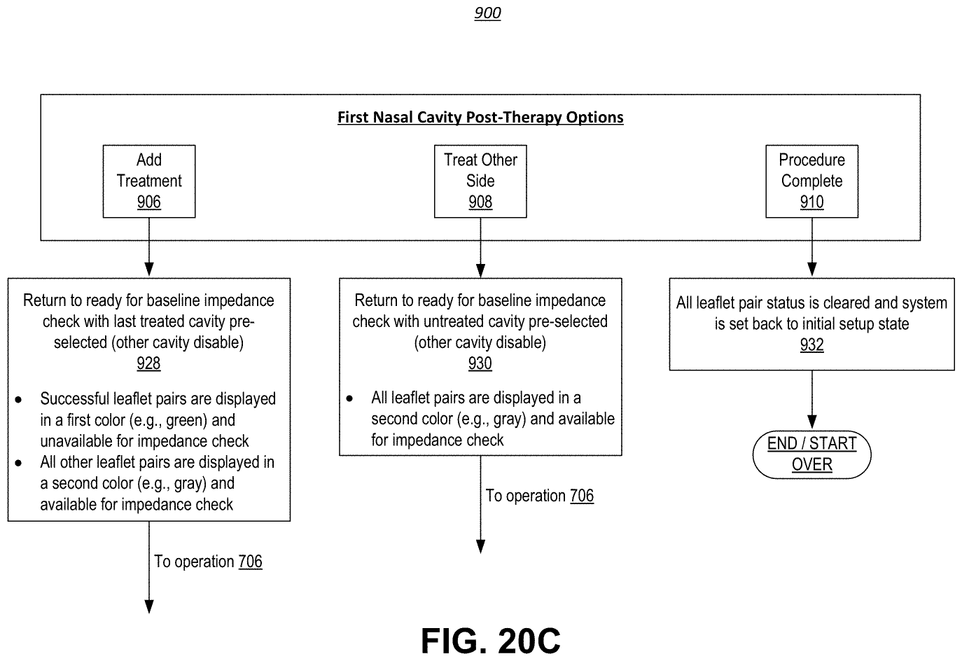

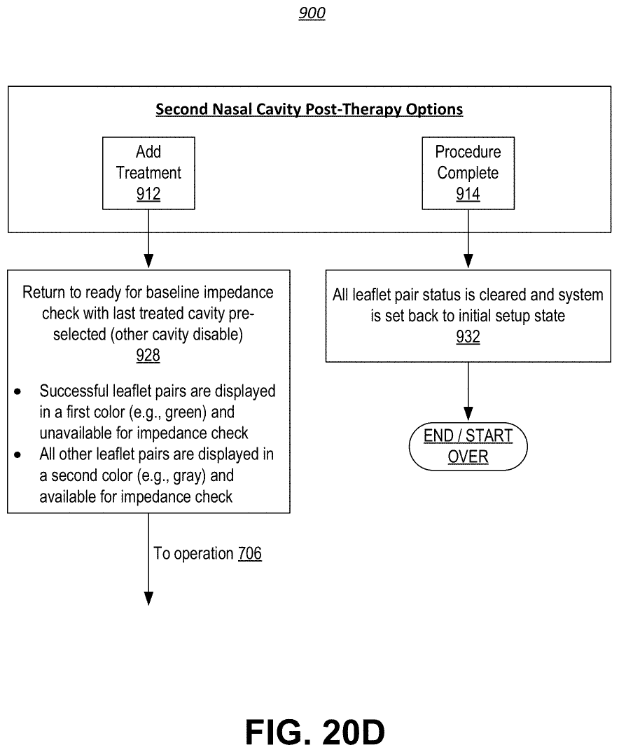

FIGS. 20A-20D show a continuous flow diagram illustrating a method 900 for post-treatment analysis. Following the delivery of energy from one or more electrodes, resulting in either successful or unsuccessful treatment of respective targeted tissue, the console unit performs post-treatment analysis. The post-treatment analysis includes a determination of any prior treatments performed, including prior use of the electrodes on prior targeted tissue for a given nasal cavity, a status of such prior use, including whether such treatment was successful or unsuccessful, and a determination of any and all further treatments to be performed. In turn, the console unit provides, via the interactive interface, one or more post-procedure inputs from which the user may select for controlling subsequent use of the treatment device to ensure that the overall procedure (i.e., treatment of rhinosinusitis) is completed by ensuring that all portions of targeted tissue undergo treatment.

For example, following the stoppage of RF therapy from the targeted energy delivery, previously described herein, the determination is made in operation 902 as to whether the other nasal cavity has already been treated. In particular, prior to initiating treatment, the user is generally provided with nasal cavity selection, in which they are able to select either the left or right nasal cavity to perform treatment on. The system is able to store such a selection and further store treatment data associated with treatment of the selected left or right nasal cavity. Accordingly, the system is able to recall, based on stored treatment data, whether only one or both of the nasal cavities have undergone treatment. If it is determined in operation 902 that the other nasal cavity has not yet been treated, then the user is presented with a set of post-therapy options for the first nasal cavity. If it is determined in operation 902 that the other nasal cavity has already been treated (i.e., both the left and right nasal cavities have undergone treatment), then the user is presented with a set of post-therapy options for the second nasal cavity.

Upon being presented with such options, a user then performs selection and confirmation of a post-therapy option (operation 904). The post-therapy options available in the event that only of the nasal cavities have been treated (i.e., the post-therapy options for the first nasal cavity) may include an option for initiating one or more additional applications of treatment to the nasal cavity just having immediately already undergone treatment (operation 906), an option for initiating application of treatment to the untreated nasal cavity (operation 908), and an option simply confirming completion of entire procedure (operation 910). The post-therapy options available in the event that both nasal cavities have been treated (i.e. post-therapy options for the second nasal cavity) may include an option for initiating one or more additional applications of treatment to the nasal cavity just having immediately undergone treatment (operation 912) and an option simply confirming completion of entire procedure (operation 914).

Referring to FIG. 20B, the post-therapy selection/confirmation process for selecting the various options is provided. A user may simply short press the handswitch to toggle between the on-screen options (operation 916), wherein an audible tone may further be provided indicating toggling between selections. The user then need only press and hold the handswitch for a period of time (e.g., 1 second) to select the desired option (operation 918), wherein an audible tone may further be provided confirming the selection. In turn, the GUI may display a confirmation alert to the user, which may be a message requesting that the user confirm their selection via confirm/cancel inputs (operation 920). Again, a user may simply short press the handswitch to toggle between the on-screen options of confirm/cancel inputs (operation 922), wherein an audible tone may further be provided indicating toggling between selections. The user then need only press and hold the handswitch for a period of time (e.g., 1 second) to select the desired confirm/cancel input (operation 924), wherein an audible tone may further be provided confirming the selection. A determination is made in operation 926 as to whether the user selected the confirm or cancel input. If it is determined in operation 926 that the cancel input is selected, than the method cycles back to a display of the post-therapy options from which the user may select. If it is determined in operation 926 that the confirm input is selected, then the system proceeds with the user selection of one of the options from either of the first cavity and second cavity post-therapy options (i.e., operations 906, 908, 910, 912, or 914).

FIG. 20C illustrates a flow diagram showing the post-therapy options available in the event that only of the nasal cavities have been treated (i.e., the post-therapy options for the first nasal cavity) and the subsequent pathways of operation. In the event that the user selects the additional treatment option (operation 906), the system returns the user to the availability assessment procedures (i.e., the baseline impedance check) with the last treated nasal cavity that was selected (operation 928), and continues back to operation 706. In the event that the user selects to treat the other side (operation 908), the system returns the user to the availability assessment procedures (i.e., the baseline impedance check) with the untreated nasal cavity selected (operation 930), and thus returns to operation 706. In the event that user selects that the procedure is complete (operation 910), all leaflet pair status is cleared and the system is set back to the initial setup state (operation 932) and the therapy procedure ends. The GUI will display an alert to the user indicating that the procedure is complete and that the user should disconnect the device.

FIG. 20D illustrates a flow diagram showing the post-therapy options available in the event that both of the nasal cavities have been treated (i.e., the post-therapy options for the second nasal cavity) and the subsequent pathways of operation. In the event that the user selects the additional treatment option (operation 912), the system returns the user to the availability assessment procedures (i.e., the baseline impedance check) with the last treated nasal cavity that was selected (operation 928), and continues back to operation 706. In the event that user selects that the procedure is complete (operation 914), all leaflet pair status is cleared and the system is set back to the initial setup state (operation 932) and the therapy procedure ends. The GUI will display an alert to the user indicating that the procedure is complete and that the user should disconnect the device.

The following provides a detailed description of the various capabilities of systems and methods of the present invention, including, but not limited to, neuromodulation monitoring, feedback, and mapping capabilities, which, in turn, allowing for detection of anatomical structures and function, neural identification and mapping, and anatomical mapping, for example.

### Neuromodulation Monitoring, Feedback, and Mapping Capabilities

As previously described, the system 100 includes a console 104 to which the device 102 is to be connected. The console 104 is configured to provide various functions for the device 102, which may include, but is not limited to, controlling, monitoring, supplying, and/or otherwise supporting operation of the device 102. The console 104 can further be configured to generate a selected form and/or magnitude of energy for delivery to tissue or nerves at the target site via the end effector (214, 314), and therefore the console 104 may have different configurations depending on the treatment modality of the device 102. For example, when device 102 is configured for electrode-based, heat-element-based, and/or transducer-based treatment, the console 104 includes an energy generator 106 configured to generate RF energy (e.g., monopolar, bipolar, or multi-polar RF energy), pulsed electrical energy, microwave energy, optical energy, ultrasound energy (e.g., intraluminally-delivered ultrasound and/or HIFU), direct heat energy, radiation (e.g., infrared, visible, and/or gamma radiation), and/or another suitable type of energy. When the device 102 is configured for cryotherapeutic treatment, the console 104 can include a refrigerant reservoir (not shown), and can be configured to supply the device 102 with refrigerant. Similarly, when the device 102 is configured for chemical-based treatment (e.g., drug infusion), the console 104 can include a chemical reservoir (not shown) and can be configured to supply the device 102 with one or more chemicals.

In some embodiments, the console 104 may include a controller 107 communicatively coupled to the device 102. However, in the embodiments described herein, the controller 107 may generally be carried by and provided within the handle 118 of the device 102. The controller 107 is configured to initiate, terminate, and/or adjust operation of one or more electrodes provided by the end effector (214, 314) directly and/or via the console 104. For example, the controller 107 can be configured to execute an automated control algorithm and/or to receive control instructions from an operator (e.g., surgeon or other medical professional or clinician). For example, the controller 107 and/or other components of the console 104 (e.g., processors, memory, etc.) can include a computer-readable medium carrying instructions, which when executed by the controller 107, causes the device 102 to perform certain functions (e.g., apply energy in a specific manner, detect impedance, detect temperature, detect nerve locations or anatomical structures, perform nerve mapping, etc.). A memory includes one or more of various hardware devices for volatile and non-volatile storage, and can include both read-only and writable memory. For example, a memory can comprise random access memory (RAM), CPU registers, read-only memory (ROM), and writable non-volatile memory, such as flash memory, hard drives, floppy disks, CDs, DVDs, magnetic storage devices, tape drives, device buffers, and so forth. A memory is not a propagating signal divorced from underlying hardware; a memory is thus non-transitory.

The console 104 may further be configured to provide feedback to an operator before, during, and/or after a treatment procedure via mapping/evaluation/feedback algorithms 110. For example, the mapping/evaluation/feedback algorithms 110 can be configured to provide information associated with the location of nerves at the treatment site, the location of other anatomical structures (e.g., vessels) at the treatment site, the temperature at the treatment site during monitoring and modulation, and/or the effect of the therapeutic neuromodulation on the nerves at the treatment site. In certain embodiments, the mapping/evaluation/feedback algorithm 110 can include features to confirm efficacy of the treatment and/or enhance the desired performance of the system 100. For example, the mapping/evaluation/feedback algorithm 110, in conjunction with the controller 107 and the end effector (214, 314), can be configured to monitor neural activity and/or temperature at the treatment site during therapy and automatically shut off the energy delivery when the neural activity and/or temperature reaches a predetermined threshold (e.g., a threshold reduction in neural activity, a threshold maximum temperature when applying RF energy, or a threshold minimum temperature when applying cryotherapy). In other embodiments, the mapping/evaluation/feedback algorithm 110, in conjunction with the controller 107, can be configured to automatically terminate treatment after a predetermined maximum time, a predetermined maximum impedance or resistance rise of the targeted tissue (i.e., in comparison to a baseline impedance measurement), a predetermined maximum impedance of the targeted tissue), and/or other threshold values for biomarkers associated with autonomic function. This and other information associated with the operation of the system 100 can be communicated to the operator via a display 112 (e.g., a monitor, touchscreen, user interface, etc.) on the console 104 and/or a separate display (not shown) communicatively coupled to the console 104.

In various embodiments, the end effector (214, 314) and/or other portions of the system 100 can be configured to detect various bioelectric-parameters of the tissue at the target site, and this information can be used by the mapping/evaluation/feedback algorithms 110 to determine the anatomy at the target site (e.g., tissue types, tissue locations, vasculature, bone structures, foramen, sinuses, etc.), locate neural tissue, differentiate between different types of neural tissue, map the anatomical and/or neural structure at the target site, and/or identify neuromodulation patterns of the end effector (214, 314) with respect to the patient's anatomy. For example, the end effector (214, 314) can be used to detect resistance, complex electrical impedance, dielectric properties, temperature, and/or other properties that indicate the presence of neural fibers and/or other anatomical structures in the target region. In certain embodiments, the end effector (214, 314), together with the mapping/evaluation/feedback algorithms 110, can be used to determine resistance (rather than impedance) of the tissue (i.e., the load) to more accurately identify the characteristics of the tissue. The mapping/evaluation/feedback algorithms 110 can determine resistance of the tissue by detecting the actual power and current of the load (e.g., via the electrodes (244, 336)).

In some embodiments, the system 100 provides resistance measurements with a high degree of accuracy and a very high degree of precision, such as precision measurements to the hundredths of an Ohm (e.g., 0.01Ω) for the range of 1-2000Ω. The high degree of resistance detection accuracy provided by the system 100 allows for the detection sub-microscale structures and events, including the firing of neural tissue, differences between neural tissue and other anatomical structures (e.g., blood vessels), and event different types of neural tissue. This information can be analyzed by the mapping/evaluation/feedback algorithms and/or the controller 107 and communicated to the operator via a high resolution spatial grid (e.g., on the display 112) and/or other type of display to identify neural tissue and other anatomy at the treatment site and/or indicate predicted neuromodulation regions based on the ablation pattern with respect to the mapped anatomy.

As previously described, in certain embodiments, each electrode (244, 336) can be operated independently of the other electrodes (244, 336). For example, each electrode can be individually activated and the polarity and amplitude of each electrode can be selected by an operator or a control algorithm executed by the controller 107. The selective independent control of the electrodes (244, 336) allows the end effector (214, 314) to detect information and deliver RF energy to highly customized regions. For example, a select portion of the electrodes (244, 336) can be activated to target specific neural fibers in a specific region while the other electrodes (244, 336) remain inactive. In certain embodiments, for example, electrodes (244, 336) may be activated across the portion of a strut that is adjacent to tissue at the target site, and the electrodes (244, 336) that are not proximate to the target tissue can remain inactive to avoid applying energy to non-target tissue. In addition, the electrodes (244, 336) can be individually activated to stimulate or therapeutically modulate certain regions in a specific pattern at different times (e.g., via multiplexing), which facilitates detection of anatomical parameters across a zone of interest and/or regulated therapeutic neuromodulation.

The electrodes (244, 336) can be electrically coupled to the energy generator 106 via wires (not shown) that extend from the electrodes (244, 336), through the shaft 116, and to the energy generator 106. When each of the electrodes (244, 336) is independently controlled, each electrode (244, 336) couples to a corresponding wire that extends through the shaft 116. This allows each electrode (244, 336) to be independently activated for stimulation or neuromodulation to provide precise ablation patterns and/or individually detected via the console 104 to provide information specific to each electrode (244, 336) for neural or anatomical detection and mapping. In other embodiments, multiple electrodes (244, 336) can be controlled together and, therefore, multiple electrodes (244, 336) can be electrically coupled to the same wire extending through the shaft 116. The energy generator 16 and/or components (e.g., a control module) operably coupled thereto can include custom algorithms to control the activation of the electrodes (244, 336). For example, the RF generator can deliver RF power at about 200-100 W to the electrodes (244, 336), and do so while activating the electrodes (244, 336) in a predetermined pattern selected based on the position of the end effector (214, 314) relative to the treatment site and/or the identified locations of the target nerves. In other embodiments, the energy generator 106 delivers power at lower levels (e.g., less than 1 W, 1-5 W, 5-15 W, 15-50 W, 50-150 W, etc.) for stimulation and/or higher power levels. For example, the energy generator 106 can be configured to delivery stimulating energy pulses of 1-3 W via the electrodes (244, 336) to stimulate specific targets in the tissue.

As previously described, the end effector (214, 314) can further include one or more temperature sensors disposed on the struts and/or other portions of the end effector (214, 314) and electrically coupled to the console 104 via wires (not shown) that extend through the shaft 116. In various embodiments, the temperature sensors can be positioned proximate to the electrodes (244, 336) to detect the temperature at the interface between tissue at the target site and the electrodes (244, 336). In other embodiments, the temperature sensors can penetrate the tissue at the target site (e.g., a penetrating thermocouple) to detect the temperature at a depth within the tissue. The temperature measurements can provide the operator or the system with feedback regarding the effect of the therapeutic neuromodulation on the tissue. For example, in certain embodiments the operator may wish to prevent or reduce damage to the tissue at the treatment site, and therefore the temperature sensors can be used to determine if the tissue temperature reaches a predetermined threshold for irreversible tissue damage. Once the threshold is reached, the application of therapeutic neuromodulation energy can be terminated to allow the tissue to remain intact and avoid significant tissue sloughing during wound healing. In certain embodiments, the energy delivery can automatically terminate based on the mapping/evaluation/feedback algorithm 110 stored on the console 104 operably coupled to the temperature sensors.

In certain embodiments, the system 100 can determine the locations and/or morphology of neural tissue and/or other anatomical structures before therapy such that the therapeutic neuromodulation can be applied to precise regions including target neural tissue, while avoiding negative effects on non-target structures, such as blood vessels. As described in further detail below, the system 100 can detect various bioelectrical parameters in an interest zone to determine the location and morphology of various neural tissue (e.g., different types of neural tissue, neuronal directionality, etc.) and/or other tissue (e.g., glandular structures, vessels, bony regions, etc.). In some embodiments, the system 100 is configured to measure bioelectric potential. To do so, one or more of the electrodes (244, 336) is placed in contact with an epithelial surface at a region of interest (e.g., a treatment site). Electrical stimuli (e.g., constant or pulsed currents at one or more frequencies) are applied to the tissue by one or more electrodes (244, 336) at or near the treatment site, and the voltage and/or current differences at various different frequencies between various pairs of electrodes (244, 336) of the end effector (214, 314) may be measured to produce a spectral profile or map of the detected bioelectric potential, which can be used to identify different types of tissues (e.g., vessels, neural tissue, and/or other types of tissue) in the region of interest. For example, current (i.e., direct or alternating current) can be applied to a pair of electrodes (244, 336) adjacent to each other and the resultant voltages and/or currents between other pairs of adjacent electrodes (244, 336) are measured. It will be appreciated that the current injection electrodes (244, 336) and measurement electrodes (244, 336) need not be adjacent, and that modifying the spacing between the two current injection electrodes (244, 336) can affect the depth of the recorded signals. For example, closely-spaced current injection electrodes (244, 336) provided recorded signals associated with tissue deeper from the surface of the tissue than further spaced apart current injection electrodes (244, 336) that provide recorded signals associated with tissue at shallower depths. Recordings from electrode pairs with different spacings may be merged to provide additional information on depth and localization of anatomical structures.

Further, complex impedance and/or resistance measurements of the tissue at the region of interest can be detected directly from current-voltage data provided by the bioelectric measurements while differing levels of frequency currents are applied to the tissue (e.g., via the end effector (214, 314)), and this information can be used to map the neural and anatomical structures by the use of frequency differentiation reconstruction. Applying the stimuli at different frequencies will target different stratified layers or cellular bodies or clusters. At high signal frequencies (e.g., electrical injection or stimulation), for example, cell membranes of the neural tissue do not impede current flow, and the current passes directly through the cell membranes. In this case, the resultant measurement (e.g., impedance, resistance, capacitance, and/or induction) is a function of the intracellular and extracellular tissue and liquids, ions, proteins and polysaccharides. At low signal frequencies, the membranes impede current flow to provide different defining characteristics of the tissues, such as the shapes and morphologies of the cells or cell densities or cell spacing. The stimulation frequencies can be in the megahertz range, in the kilohertz range (e.g., 400-500 kHz, 450-480 kHz, etc.), and/or other frequencies attuned to the tissue being stimulated and the characteristics of the device being used. The detected complex impedance or resistances levels from the zone of interest can be displayed to the user (e.g., via the display 112) to visualize certain structures based on the stimulus frequency.

Further, the inherent morphology and composition of the anatomical structures in a given region or zone of the patient react differently to different frequencies and, therefore, specific frequencies can be selected to identify very specific structures. For example, the morphology or composition of targeted structures for anatomical mapping may depend on whether the cells of tissue or other structure are membranonic, stratified, and/or annular. In various embodiments, the applied stimulation signals can have predetermined frequencies attuned to specific neural tissue, such as the level of myelination and/or morphology of the myelination. For example, second axonal parasympathetic structures are poorly myelinated than sympathetic nerves or other structures and, therefore, will have a distinguishable response (e.g., complex impedance, resistance, etc.) with respect to a selected frequency than sympathetic nerves. Accordingly, applying signals with different frequencies to the target site can distinguish the targeted parasympathetic nerves from the non-targeted sensory nerves, and therefore provide highly specific target sites for neural mapping before or after therapy and/or neural evaluation post-therapy. In some embodiments, the neural and/or anatomical mapping includes measuring data at a region of interest with at least two different frequencies to identify certain anatomical structures such that the measurements are taken first based on a response to an injection signal having a first frequency and then again based on an injection signal having a second frequency different from the first. For example, there are two frequencies at which hypertrophied (i.e., disease-state characteristics) sub-mucosal targets have a different electrical conductivity or permittivity compared to “normal” (i.e., healthy) tissue. Complex conductivity may be determined based on one or more measured physiological parameters (e.g., complex impedance, resistance, dielectric measurements, dipole measurements, etc.) and/or observance of one or more confidently known attributes or signatures. Furthermore, the system 100 can also apply neuromodulation energy via the electrodes (244, 336) at one or more predetermined frequencies attuned to a target neural structure to provide highly targeted ablation of the selected neural structure associated with the frequency(ies). This highly targeted neuromodulation also reduces the collateral effects of neuromodulation therapy to non-target sites/structures (e.g., blood vessels) because the targeted signal (having a frequency tuned to a target neural structure) will not have the same modulating effects on the non-target structures.

Accordingly, bioelectric properties, such as complex impedance and resistance, can be used by the system 100 before, during, and/or after neuromodulation therapy to guide one or more treatment parameters. For example, before, during, and/or after treatment, impedance or resistance measurements may be used to confirm and/or detect contact between one or more electrodes (244, 336) and the adjacent tissue. The impedance or resistance measurements can also be used to detect whether the electrodes (244, 336) are placed appropriately with respect to the targeted tissue type by determining whether the recorded spectra have a shape consistent with the expected tissue types and/or whether serially collected spectra were reproducible. In some embodiments, impedance or resistance measurements may be used to identify a boundary for the treatment zone (e.g., specific neural tissue that are to be disrupted), anatomical landmarks, anatomical structures to avoid (e.g., vascular structures or neural tissue that should not be disrupted), and other aspects of delivering energy to tissue.

The bioelectric information can be used to produce a spectral profile or map of the different anatomical features tissues at the target site, and the anatomical mapping can be visualized in a 3D or 2D image via the display 112 and/or other user interface to guide the selection of a suitable treatment site. This neural and anatomical mapping allows the system 100 to accurately detect and therapeutically modulate the postganglionic parasympathetic neural fibers that innervate the mucosa at numerous neural entrance points within a given zone or region of a patient. Further, because there are not any clear anatomical markers denoting the location of the SPF, accessory foramen, and microforamina, the neural mapping allows the operator to identify and therapeutically modulate nerves that would otherwise be unidentifiable without intricate dissection of the mucosa. In addition, anatomical mapping also allows the clinician to identify certain structures that the clinician may wish to avoid during therapeutic neural modulation (e.g., certain arteries). The neural and anatomical bioelectric properties detected by the system 100 can also be used during and after treatment to determine the real-time effect of the therapeutic neuromodulation on the treatment site. For example, the mapping/evaluation/feedback algorithms 110 can also compare the detected neural locations and/or activity before and after therapeutic neuromodulation, and compare the change in neural activity to a predetermined threshold to assess whether the application of therapeutic neuromodulation was effective across the treatment site.

In various embodiments, the system 100 can also be configured to map the expected therapeutic modulation patterns of the electrodes (244, 336) at specific temperatures and, in certain embodiments, take into account tissue properties based on the anatomical mapping of the target site. For example, the system 100 can be configured to map the ablation pattern of a specific electrode ablation pattern at the 45° C. isotherm, the 55° C. isotherm, the 65° C. isotherm, and/or other temperature/ranges (e.g., temperatures ranging from 45° C. to 70° C. or higher) depending on the target site and/or structure.

The system 100 may provide, via the display 112, three-dimensional views of such projected ablation patterns of the electrodes (244, 336) of the end effector (214, 314). The ablation pattern mapping may define a region of influence that each electrode (244, 336) has on the surrounding tissue. The region of influence may correspond to the region of tissue that would be exposed to therapeutically modulating energy based on a defined electrode activation pattern (i.e., one, two, three, four, or more electrodes on any given strut). In other words, the ablation pattern mapping can be used to illustrate the ablation pattern of any number of electrodes (244, 336), any geometry of the electrode layout, and/or any ablation activation protocol (e.g., pulsed activation, multi-polar/sequential activation, etc.).

In some embodiments, the ablation pattern may be configured such that each electrode (244, 336) has a region of influence surrounding only the individual electrode (244, 336) (i.e., a “dot” pattern). In other embodiments, the ablation pattern may be such that two or more electrodes (244, 336) may link together to form a sub-grouped regions of influence that define peanut-like or linear shapes between two or more electrodes (244, 336). In further embodiments, the ablation pattern can result in a more expansive or contiguous pattern in which the region of influence extends along multiple electrodes (244, 336) (e.g., along each strut). In still further embodiments, the ablation pattern may result in different regions of influence depending upon the electrode activation pattern, phase angle, target temperature, pulse duration, device structure, and/or other treatment parameters. The three-dimensional views of the ablation patterns can be output to the display 112 and/or other user interfaces to allow the clinician to visualize the changing regions of influence based on different durations of energy application, different electrode activation sequences (e.g., multiplexing), different pulse sequences, different temperature isotherms, and/or other treatment parameters. This information can be used to determine the appropriate ablation algorithm for a patient's specific anatomy. In other embodiments, the three-dimensional visualization of the regions of influence can be used to illustrate the regions from which the electrodes (244, 336) detect data when measuring bioelectrical properties for anatomical mapping. In this embodiment, the three dimensional visualization can be used to determine which electrode activation pattern should be used to determine the desired properties (e.g., impedance, resistance, etc.) in the desired area. In certain embodiments, it may be better to use dot assessments, whereas in other embodiments it may be more appropriate to detect information from linear or larger contiguous regions.

In some embodiments, the mapped ablation pattern is superimposed on the anatomical mapping to identify what structures (e.g., neural tissue, vessels, etc.) will be therapeutically modulated or otherwise affected by the therapy. An image may be provided to the surgeon which includes a digital illustration of a predicted or planned neuromodulation zone in relation to previously identified anatomical structures in a zone of interest. For example, the illustration may show numerous neural tissue and, based on the predicted neuromodulation zone, identifies which neural tissue are expected to be therapeutically modulated. The expected therapeutically modulated neural tissue may be shaded to differentiate them from the non-affected neural tissue. In other embodiments, the expected therapeutically modulated neural tissue can be differentiated from the non-affected neural tissue using different colors and/or other indicators. In further embodiments, the predicted neuromodulation zone and surrounding anatomy (based on anatomical mapping) can be shown in a three dimensional view (and/or include different visualization features (e.g., color-coding to identify certain anatomical structures, bioelectric properties of the target tissue, etc.). The combined predicted ablation pattern and anatomical mapping can be output to the display 112 and/or other user interfaces to allow the clinician to select the appropriate ablation algorithm for a patient's specific anatomy.

The imaging provided by the system 100 allows the clinician to visualize the ablation pattern before therapy and adjust the ablation pattern to target specific anatomical structures while avoiding others to prevent collateral effects. For example, the clinician can select a treatment pattern to avoid blood vessels, thereby reducing exposure of the vessel to the therapeutic neuromodulation energy. This reduces the risk of damaging or rupturing vessels and, therefore, prevents immediate or latent bleeding. Further, the selective energy application provided by the neural mapping reduces collateral effects of the therapeutic neuromodulation, such as tissue sloughing off during wound healing (e.g., 1-3 weeks post ablation), thereby reducing the aspiration risk associated with the neuromodulation procedure.

The system 100 can be further configured to apply neuromodulation energy (via the electrodes (244, 336)) at specific frequencies attuned to the target neural structure and, therefore, specifically target desired neural tissue over non-target structures. For example, the specific neuromodulation frequencies can correspond to the frequencies identified as corresponding to the target structure during neural mapping. As described above, the inherent morphology and composition of the anatomical structures react differently to different frequencies. Thus, frequency-tuned neuromodulation energy tailored to a target structure does not have the same modulating effects on non-target structures. More specifically, applying the neuromodulation energy at the target-specific frequency causes ionic agitation in the target neural structure, leading to differentials in osmotic potentials of the targeted neural tissue and dynamic changes in neuronal membronic potentials (resulting from the difference in intra-cellular and extra-cellular fluidic pressure). This causes degeneration, possibly resulting in vacuolar degeneration and, eventually, necrosis at the target neural structure, but is not expected to functionally affect at least some non-target structures (e.g., blood vessels). Accordingly, the system 100 can use the neural-structure specific frequencies to both (1) identify the locations of target neural tissue to plan electrode ablation configurations (e.g., electrode geometry and/or activation pattern) that specifically focus the neuromodulation on the target neural structure; and (2) apply the neuromodulation energy at the characteristic neural frequencies to selectively ablate the neural tissue responsive to the characteristic neural frequencies. For example, the end effector (214, 314) of the system 100 may selectively stimulate and/or modulate parasympathetic fibers, sympathetic fibers, sensory fibers, alpha/beta/delta fibers, C-fibers, anoxic terminals of one or more of the foregoing, insulated over non-insulated fibers (regions with fibers), and/or other neural tissue. In some embodiments, the system 100 may also selectively target specific cells or cellular regions during anatomical mapping and/or therapeutic modulation, such as smooth muscle cells, sub-mucosal glands, goblet cells, and stratified cellular regions within a given tissue type. Therefore, the system 100 provides highly selective neuromodulation therapy specific to targeted neural tissue, and reduces the collateral effects of neuromodulation therapy to non-target structures (e.g., blood vessels).

The present disclosure provides a method of anatomical mapping and therapeutic neuromodulation. The method includes expanding an end effector (i.e., end effector (214, 314)) at a zone of interest (“interest zone”). For example, the end effector (214, 314) can be expanded such that at least some of the electrodes (244, 336) are placed in contact with tissue at the interest zone. The expanded device can then take bioelectric measurements via the electrodes (244, 336) and/or other sensors to ensure that the desired electrodes are in proper contact with the tissue at the interest zone. In some embodiments, for example, the system 100 detects the impedance and/or resistance across pairs of the electrodes (244, 336) to confirm that the desired electrodes have appropriate surface contact with the tissue and that all of the electrodes are (244, 336) functioning properly.

The method continues by optionally applying an electrical stimulus to the tissue, and detecting bioelectric properties of the tissue to establish baseline norms of the tissue. For example, the method can include measuring resistance, complex impedance, current, voltage, nerve firing rate, neuromagnetic field, muscular activation, and/or other parameters that are indicative of the location and/or function of neural tissue and/or other anatomical structures (e.g., glandular structures, blood vessels, etc.). In some embodiments, the electrodes (244, 336) send one or more stimulation signals (e.g., pulsed signals or constant signals) to the interest zone to stimulate neural activity and initiate action potentials. The stimulation signal can have a frequency attuned to a specific target structure (e.g., a specific neural structure, a glandular structure, a vessel) that allows for identification of the location of the specific target structure. The specific frequency of the stimulation signal is a function of the host permeability and, therefore, applying the unique frequency alters the tissue attenuation and the depth into the tissue the RF energy will penetrate. For example, lower frequencies typically penetrate deeper into the tissue than higher frequencies.

Pairs of the non-stimulating electrodes (244, 336) of the end effector (214, 314) can then detect one or more bioelectric properties of the tissue that occur in response to the stimulus, such as impedance or resistance. For example, an array of electrodes (e.g., the electrodes (244, 336)) can be selectively paired together in a desired pattern (e.g., multiplexing the electrodes (244, 336)) to detect the bioelectric properties at desired depths and/or across desired regions to provide a high level of spatial awareness at the interest zone. In certain embodiments, the electrodes (244, 336) can be paired together in a time-sequenced manner according to an algorithm (e.g., provided by the mapping/evaluation/feedback algorithms 110). In various embodiments, stimuli can be injected into the tissue at two or more different frequencies, and the resultant bioelectric responses (e.g., action potentials) in response to each of the injected frequencies can be detected via various pairs of the electrodes (244, 336). For example, an anatomical or neural mapping algorithm can cause the end effector (214, 314) to deliver pulsed RF energy at specific frequencies between different pairs of the electrodes (244, 336) and the resultant bioelectric response can be recorded in a time sequenced rotation until the desired interest zone is adequately mapped (i.e., “multiplexing”). For example, the end effector (214, 314) can deliver stimulation energy at a first frequency via adjacent pairs of the electrodes (244, 336) for a predetermined time period (e.g., 1-50 milliseconds), and the resultant bioelectric activity (e.g., resistance) can be detected via one or more other pairs of electrodes (244, 336) (e.g., spaced apart from each other to reach varying depths within the tissue). The end effector (214, 314) can then apply stimulation energy at a second frequency different from the first frequency, and the resultant bioelectric activity can be detected via the other electrodes. This can continue when the interest zone has been adequately mapped at the desired frequencies. As described in further detail below, in some embodiments the baseline tissue bioelectric properties (e.g., nerve firing rate) are detected using static detection methods (without the injection of a stimulation signal).

After detecting the baseline bioelectric properties, the information can be used to map anatomical structures and/or functions at the interest zone. For example, the bioelectric properties detected by the electrodes (244, 336) can be amazed via the mapping/evaluation/feedback algorithms 110, and an anatomical map can be output to a user via the display 112. In some embodiments, complex impedance, dielectric, or resistance measurements can be used to map parasympathetic nerves and, optionally, identify neural tissue in a diseased state of hyperactivity. The bioelectric properties can also be used to map other non-target structures and the general anatomy, such as blood vessels, bone, and/or glandular structures. The anatomical locations can be provided to a user (e.g., on the display 112) as a two-dimensional map (e.g., illustrating relative intensities, illustrating specific sites of potential target structures) and/or as a three-dimensional image. This information can be used to differentiate structures on a submicron, cellular level and identify very specific target structures (e.g., hyperactive parasympathetic nerves). The method can also predict the ablation patterns of the end effector (214, 314) based on different electrode neuromodulation protocol and, optionally, superimpose the predicted neuromodulation patterns onto the mapped anatomy to indicate to the user which anatomical structures will be affected by a specific neuromodulation protocol. For example, when the predicted neuromodulation pattern is displayed in relation to the mapped anatomy, a clinician can determine whether target structures will be appropriately ablated and whether non-target structures (e.g., blood vessels) will be undesirably exposed to the therapeutic neuromodulation energy. Thus, the method can be used for planning neuromodulation therapy to locate very specific target structures, avoid non-target structures, and select electrode neuromodulation protocols.

Once the target structure is located and a desired electrode neuromodulation protocol has been selected, the method continues by applying therapeutic neuromodulation to the target structure. The neuromodulation energy can be applied to the tissue in a highly targeted manner that forms micro-lesions to selectively modulate the target structure, while avoiding non-targeted blood vessels and allowing the surrounding tissue structure to remain healthy for effective wound healing. In some embodiments, the neuromodulation energy can be applied in a pulsed manner, allowing the tissue to cool between modulation pulses to ensure appropriate modulation without undesirably affecting non-target tissue. In some embodiments, the neuromodulation algorithm can deliver pulsed RF energy between different pairs of the electrodes (244, 336) in a time sequenced rotation until neuromodulation is predicted to be complete (i.e., “multiplexing”). For example, the end effector (214, 314) can deliver neuromodulation energy (e.g., having a power of 5-10 W (e.g., 7 W, 8 W, 9 W) and a current of about 50-100 mA) via adjacent pairs of the electrodes (244, 336) until at least one of the following conditions is met: (a) load resistance reaches a predefined maximum resistance (e.g., 350Ω); (b) a thermocouple temperature associated with the electrode pair reaches a predefined maximum temperature (e.g., 80° C.); or (c) a predetermined time period has elapsed (e.g., 10 seconds). After the predetermined conditions are met, the end effector (214, 314) can move to the next pair of electrodes in the sequence, and the neuromodulation algorithm can terminate when all of the load resistances of the individual pairs of electrodes is at or above a predetermined threshold (e.g., 100Ω). In various embodiments, the RF energy can be applied at a predetermined frequency (e.g., 450-500 kHz) and is expected to initiate ionic agitation of the specific target structure, while avoiding functional disruption of non-target structures.

During and/or after neuromodulation therapy, the method continues by detecting and, optionally, mapping the post-therapy bioelectric properties of the target site. This can be performed in a similar manner as described above. The post-therapy evaluation can indicate if the target structures (e.g., hyperactive parasympathetic nerves) were adequately modulated or ablated. If the target structures are not adequately modulated (i.e., if neural activity is still detected in the target structure and/or the neural activity has not decreased), the method can continue by again applying therapeutic neuromodulation to the target. If the target structures were adequately ablated, the neuromodulation procedure can be completed.

### Detection of Anatomical Structures and Function

Various embodiments of the present technology can include features that measure bio-electric, dielectric, and/or other properties of tissue at target sites to determine the presence, location, and/or activity of neural tissue and other anatomical structures and, optionally, map the locations of the detected neural tissue and/or other anatomical structures. For example, the present technology can be used to detect glandular structures and, optionally, their mucoserous functions and/or other functions. The present technology can also be configured to detect vascular structures (e.g., arteries) and, optionally, their arterial functions, volumetric pressures, and/or other functions. The mapping features discussed below can be incorporated into any the system 100 and/or any other devices disclosed herein to provide an accurate depiction of nerves at the target site.

Neural and/or anatomical detection can occur (a) before the application of a therapeutic neuromodulation energy to determine the presence or location of neural tissue and other anatomical structures (e.g., blood vessels, glands, etc.) at the target site and/or record baseline levels of neural activity; (b) during therapeutic neuromodulation to determine the real-time effect of the energy application on the neural fibers at the treatment site; and/or (c) after therapeutic neuromodulation to confirm the efficacy of the treatment on the targeted structures (e.g., nerves glands, etc.). This allows for the identification of very specific anatomical structures (even to the micro-scale or cellular level) and, therefore, provides for highly targeted neuromodulation. This enhances the efficacy and efficiency of the neuromodulation therapy. In addition, the anatomical mapping reduces the collateral effects of neuromodulation therapy to non-target sites.

Accordingly, the targeted neuromodulation inhibits damage or rupture of blood vessels (i.e., inhibits undesired bleeding) and collateral damage to tissue that may be of concern during wound healing (e.g., when damaged tissue sloughs off).

In certain embodiments, the systems disclosed herein can use bioelectric measurements, such as impedance, resistance, voltage, current density, and/or other parameters (e.g., temperature) to determine the anatomy, in particular the neural, glandular, and vascular anatomy, at the target site. The bioelectric properties can be detected after the transmission of a stimulus (e.g., an electrical stimulus, such as RF energy delivered via the electrodes (244, 336); i.e., “dynamic” detection) and/or without the transmission of a stimulus (i.e., “static” detection). Dynamic measurements include various embodiments to excite and/or detect primary or secondary effects of neural activation and/or propagation. Such dynamic embodiments involve the heightened states of neural activation and propagation and use this dynamic measurement for nerve location and functional identification relative to the neighboring tissue types. For example, a method of dynamic detection can include: (1) delivering stimulation energy to a treatment site via a treatment device (e.g., the end effector) to excite parasympathetic nerves at the treatment site; (2) measuring one or more physiological parameters (e.g., resistance, impedance, etc.) at the treatment site via a measuring/sensing array of the treatment device (e.g., the electrodes (244, 336)); (4) based on the measurements, identifying the relative presence and position of parasympathetic nerves at the treatment site; and (5) delivering ablation energy to the identified parasympathetic nerves to block the detected para-sympathetic nerves.

Static measurements include various embodiments associated with specific native properties of the stratified or cellular composition at or near the treatment site. The static embodiments are directed to inherent biologic and electrical properties of tissue types at or near the treatment site, the stratified or cellular compositions at or near the treatment site, and contrasting both foregoing measurements with tissue types adjacent the treatment site (and that are not targeted for neuromodulation). This information can be used to localize specific targets (e.g., parasympathetic fibers) and non-targets (e.g., vessels, sensory nerves, etc.). For example, a method of static detection can include: (1) before ablation, utilizing a measuring/sensing array of a treatment device (e.g., the electrodes (244, 336)) to determine one or more baseline physiological parameters; (2) geometrically identifying inherent tissue properties within a region of interest based on the measured physiological parameters (e.g., resistance, impedance, etc.); (3) delivering ablation energy to one or more nerves within the region of via treatment device interest; (4) during the delivery of the ablation energy, determining one or more mid-procedure physiological parameters via the measuring/sensing array; and (5) after the delivery of ablation energy, determining one or more post-procedure physiological parameters via the measurement/sensing array to determine the effectiveness of the delivery of the ablation energy on blocking the nerves that received the ablation energy.

After the initial static and/or dynamic detection of bioelectric properties, the location of anatomical features can be used to determine where the treatment site(s) should be with respect to various anatomical structures for therapeutically effective neuromodulation of the targeted nerves. The bioelectric and other physiological properties described herein can be detected via electrodes (e.g., the electrodes (244, 336) of the end effector (214, 314)), and the electrode pairings on a device (e.g., end effector (214, 314)) can be selected to obtain the bioelectric data at specific zones or regions and at specific depths of the targeted regions. The specific properties detected at or surrounding target neuromodulation sites and associated methods for obtaining these properties are described below. These specific detection and mapping methods discussed below are described with reference to the system 100, although the methods can be implemented on other suitable systems and devices that provide for anatomical identification, anatomical mapping and/or neuromodulation therapy.

### Neural Identification and Mapping

In many neuromodulation procedures, it is beneficial to identify the portions of the nerves that fall within a zone and/or region of influence (referred to as the “interest zone”) of the energy delivered by a device 102, as well as the relative three-dimensional position of the neural tissue relative to the device 102. Characterizing the portions of the neural tissue within the interest zone and/or determining the relative positions of the neural tissue within the interest zone enables the clinician to (1) selectively activate target neural tissue over non-target structures (e.g., blood vessels), and (2) sub-select specific targeted neural tissue (e.g., parasympathetic nerves) over non-target neural tissue (e.g., sensory nerves, subgroups of neural tissue, neural tissue having certain compositions or morphologies). The target structures (e.g., parasympathetic nerves) and non-target structures (e.g., blood vessels, sensory nerves, etc.) can be identified based on the inherent signatures of specific structures, which are defined by the unique morphological compositions of the structures and the bioelectrical properties associated with these morphological compositions. For example, unique, discrete frequencies can be associated with morphological compositions and, therefore, be used to identify certain structures. The target and non-target structures can also be identified based on relative bioelectrical activation of the structures to sub-select specific neural structures. Further, target and non-target structures can be identified by the differing detected responses of the structures to a tailored injected stimuli. For example, the systems described herein can detect the magnitude of response of structures and the difference in the responses of anatomical structures with respect to differing stimuli (e.g., stimuli injected at different frequencies).

At least for purposes of this disclosure, a nerve can include the following portions that are defined based on their respective orientations relative to the interest zone: terminating neural tissue (e.g., terminating axonal structures), branching neural tissue (e.g., branching axonal structures), and travelling neural tissue (e.g., travelling axonal structures). For example, terminating neural tissue enter the zone but do not exit. As such, terminating neural tissue are terminal points for neuronal signaling and activation. Branching neural tissue are nerves that enter the interest zone and increase number of nerves exiting the interest zone. Branching neural tissue are typically associated with a reduction in relative geometry of nerve bundle. Travelling neural tissue are nerves that enter the interest zone and exit the zone with no substantially no change in geometry or numerical value.

The system 100 can be used to detect voltage, current, complex impedance, resistance, permittivity, and/or conductivity, which are tied to the compound action potentials of nerves, to determine and/or map the relative positions and proportionalities of nerves in the interest zone. Neuronal cross-sectional area (“CSA”) is expected to be due to the increase in axonic structures. Each axon is a standard size. Larger nerves (in cross-sectional dimension) have a larger number of axons than nerves having smaller cross-sectional dimensions. The compound action responses from the larger nerves, in both static and dynamic assessments, are greater than smaller nerves. This is at least in part because the compound action potential is the cumulative action response from each of the axons. When using static analysis, for example, the system 100 can directly measure and map impedance or resistance of nerves and, based on the determined impedance or resistance, determine the location of nerves and/or relative size of the nerves. In dynamic analysis, the system 100 can be used to apply a stimulus to the interest zone and detect the dynamic response of the neural tissue to the stimulus. Using this information, the system 100 can determine and/or map impedance or resistance in the interest zone to provide information related to the neural positions or relative nerve sizes. Neural impedance mapping can be illustrated by showing the varying complex impedance levels at a specific location at differing cross-sectional depths. In other embodiments, neural impedance or resistance can be mapped in a three-dimensional display.

Identifying the portions and/or relative positions of the nerves within the interest zone can inform and/or guide selection of one or more treatment parameters (e.g., electrode ablation patterns, electrode activation plans, etc.) of the system 100 for improving treatment efficiency and efficacy. For example, during neural monitoring and mapping, the system 100 can identify the directionality of the nerves based at least in part on the length of the neural structure extending along the interest zone, relative sizing of the neural tissue, and/or the direction of the action potentials. This information can then be used by the system 100 or the clinician to automatically or manually adjust treatment parameters (e.g., selective electrode activation, bipolar and/or multipolar activation, and/or electrode positioning) to target specific nerves or regions of nerves. For example, the system 100 can selectively activate specific electrodes (244, 336), electrode combinations (e.g., asymmetric or symmetric), and/or adjust the bi-polar or multi-polar electrode configuration. In some embodiments, the system 100 can adjust or select the waveform, phase angle, and/or other energy delivery parameters based on the nerve portion/position mapping and/or the nerve proportionality mapping. In some embodiments, structure and/or properties of the electrodes (244, 336) themselves (e.g., material, surface roughening, coatings, cross-sectional area, perimeter, penetrating, penetration depth, surface-mounted, etc.) may be selected based on the nerve portion and proportionality mapping.

In various embodiments, treatment parameters and/or energy delivery parameters can be adjusted to target on-axis or near axis travelling neural tissue and/or avoid the activation of traveling neural tissue that are at least generally perpendicular to the end effector (214, 314). Greater portions of the on-axis or near axis travelling neural tissue are exposed and susceptible to the neuromodulation energy provided by the end effector (214, 314) than a perpendicular travelling neural structure, which may only be exposed to therapeutic energy at a discrete cross-section. Therefore, the end effector (214, 314) is more likely to have a greater effect on the on-axis or near axis travelling neural tissue. The identification of the neural structure positions (e.g., via complex impedance or resistance mapping) can also allow targeted energy delivery to travelling neural tissue rather than branching neural tissue (typically downstream of the travelling neural tissue) because the travelling neural tissue are closer to the nerve origin and, therefore, more of the nerve is affected by therapeutic neuromodulation, thereby resulting in a more efficient treatment and/or a higher efficacy of treatment. Similarly, the identification of neural structure positions can be used to target travelling and branching neural tissue over terminal neural tissue. In some embodiments, the treatment parameters can be adjusted based on the detected neural positions to provide a selective regional effect. For example, a clinician can target downstream portions of the neural tissue if only wanting to influence partial effects on very specific anatomical structures or positions.

In various embodiments, neural locations and/or relative positions of nerves can be determined by detecting the nerve-firing voltage and/or current over time. An array of the electrodes (244, 336) can be positioned in contact with tissue at the interest zone, and the electrodes (244, 336) can measure the voltage and/or current associated with nerve-firing. This information can optionally be mapped (e.g., on a display 112) to identify the location of nerves in a hyper state (i.e., excessive parasympathetic tone). Rhinitis is at least in part the result of over-firing nerves because this hyper state drives the hyper-mucosal production and hyper-mucosal secretion. Therefore, detection of nerve firing rate via voltage and current measurements can be used to locate the portions of the interest region that include hyper-parasympathetic neural function (i.e., nerves in the diseased state). This allows the clinician to locate specific nerves (i.e., nerves with excessive parasympathetic tone) before neuromodulation therapy, rather than simply targeting all parasympathetic nerves (including non-diseased state parasympathetic nerves) to ensure that the correct tissue is treated during neuromodulation therapy. Further, nerve firing rate can be detected during or after neuromodulation therapy so that the clinician can monitor changes in nerve firing rate to validate treatment efficacy. For example, recording decreases or elimination of nerve firing rate after neuromodulation therapy can indicate that the therapy was effective in therapeutically treating the hyper/diseased nerves.

In various embodiments, the system 100 can detect neural activity using dynamic activation by injecting a stimulus signal (i.e., a signal that temporarily activates nerves) via one or more of the electrodes (244, 336) to induce an action potential, and other pairs of electrodes (244, 336) can detect bioelectric properties of the neural response. Detecting neural tissue using dynamic activation involves detecting the locations of action potentials within the interest zone by measuring the discharge rate in neurons and the associated processes. The ability to numerically measure, profile, map, and/or image fast neuronal depolarization for generating an accurate index of activity is a factor in measuring the rate of discharge in neurons and their processes. The action potential causes a rapid increase in the voltage across nerve fiber and the electrical impulse then spreads along the fiber. As an action potential occurs, the conductance of a neural cell membrane changes, becoming about 40 times larger than it is when the cell is at rest. During the action potential or neuronal depolarization, the membrane resistance diminishes by about 80 times, thereby allowing an applied current to enter the intracellular space as well. Over a population of neurons, this leads to a net decrease in the resistance during coherent neuronal activity, such as chronic para-sympathetic responses, as the intracellular space will provide additional conductive ions. The magnitude of such fast changes has been estimated to have local resistivity changes with recording near DC is 2.8-3.7% for peripheral nerve bundles.

Detecting neural tissue using dynamic activation includes detecting the locations of action potentials within the interest zone by measuring the discharge rate in neurons and the associated processes. The basis of each this discharge is the action potential, during which there is a depolarization of the neuronal membrane of up to 110 mV or more, lasting approximately 2 milliseconds, and due to the transfer of micromolar quantities of ions (e.g., sodium and potassium) across the cellular membrane. The complex impedance or resistance change due to the neuronal membrane falls from 1000 to 25 Ωcm. The introduction of a stimulus and subsequent measurement of the neural response can attenuate noise and improve signal to noise ratios to precisely focus on the response region to improve neural detection, measurement, and mapping.

In some embodiments, the difference in measurements of physiological parameters (e.g., complex impedance, resistance, voltage) over time, which can reduce errors, can be used to create a neural profiles, spectrums, or maps. For example, the sensitivity of the system 100 can be improved because this process provides repeated averaging to a stimulus. As a result, the mapping function outputs can be a unit-less ratio between the reference and test collated data at a single frequency and/or multiple frequencies and/or multiple amplitudes. Additional considerations may include multiple frequency evaluation methods that consequently expand the parameter assessments, such as resistivity, admittivity, center frequency, or ratio of extra- to intracellular resistivity.

In some embodiments, the system 100 may also be configured to indirectly measure the electrical activity of neural tissue to quantify the metabolic recovery processes that accompany action potential activity and act to restore ionic gradients to normal. These are related to an accumulation of ions in the extracellular space. The indirect measurement of electrical activity can be approximately a thousand times larger (in the order of millimolar), and thus are easier to measure and can enhance the accuracy of the measured electrical properties used to generate the neural maps.

The system 100 can perform dynamic neural detection by detecting nerve-firing voltage and/or current and, optionally, nerve firing rate over time, in response to an external stimulation of the nerves. For example, an array of the electrodes (244, 336) can be positioned in contact with tissue at the interest zone, one or more of the electrodes (244, 336) can be activated to inject a signal into the tissue that stimulates the nerves, and other electrodes (244, 336) of the electrode array can measure the neural voltage and/or current due to nerve firing in response to the stimulus. This information can optionally be mapped (e.g., on a display 112) to identify the location of nerves and, in certain embodiments, identify parasympathetic nerves in a hyper state (e.g., indicative of Rhinitis or other diseased state). The dynamic detection of neural activity (voltage, current, firing rate, etc.) can be performed before neuromodulation therapy to detect target nerve locations to select the target site and treatment parameters to ensure that the correct tissue is treated during neuromodulation therapy. Further, dynamic detection of neural activity can be performed during or after neuromodulation therapy to allow the clinician to monitor changes in neural activity to validate treatment efficacy. For example, recording decreases or elimination of neural activity after neuromodulation therapy can indicate that the therapy was effective in therapeutically treating the hyper/diseased nerves.

In some embodiments, a stimulating signal can be delivered to the vicinity of the targeted nerve via one or more penetrating electrodes (e.g., microneedles that penetrate tissue) associated with the end effector (214, 314) and/or a separate device. The stimulating signal generates an action potential, which causes smooth muscle cells or other cells to contract. The location and strength of this contraction can be detected via the penetrating electrode(s) and, thereby, indicate to the clinician the distance to the nerve and/or the location of the nerve relative to the stimulating needle electrode. In some embodiments, the stimulating electrical signal may have a voltage of typically 1-2 mA or greater and a pulse width of typically 100-200 microseconds or greater. Shorter pulses of stimulation result in better discrimination of the detected contraction, but may require more current. The greater the distance between the electrode and the targeted nerve, the more energy is required to stimulate. The stimulation and detection of contraction strength and/or location enables identification of how close or far the electrodes are from the nerve, and therefore can be used to localize the nerve spatially. In some embodiments, varying pulse widths may be used to measure the distance to the nerve. As the needle becomes closer to the nerve, the pulse duration required to elicit a response becomes less and less.

To localize nerves via muscle contraction detection, the system 100 can vary pulse-width or amplitude to vary the energy (Energy=pulse-width*amplitude) of the stimulus delivered to the tissue via the penetrating electrode(s). By varying the stimulus energy and monitoring muscle contraction via the penetrating electrodes and/or other type of sensor, the system 100 can estimate the distance to the nerve. If a large amount of energy is required to stimulate the nerve/contract the muscle, the stimulating/penetrating electrode is far from the nerve. As the stimulating/penetrating electrode, moves closer to the nerve, the amount of energy required to induce muscle contraction will drop. For example, an array of penetrating electrodes can be positioned in the tissue at the interest zone and one or more of the electrodes can be activated to apply stimulus at different energy levels until they induce muscle contraction. Using an iterative process, localize the nerve (e.g., via the mapping/evaluation/feedback algorithm 110).

In some embodiments, the system 100 can measure the muscular activation from the nerve stimulus (e.g., via the electrodes (244, 336)) to determine neural positioning for neural mapping, without the use of penetrating electrodes. In this embodiment, the treatment device targets the smooth muscle cells' varicosities surrounding the submucosal glands and the vascular supply, and then the compound muscle action potential. This can be used to summate voltage response from the individual muscle fiber action potentials. The shortest latency is the time from stimulus artifact to onset of the response. The corresponding amplitude is measured from baseline to negative peak and measured in millivolts (mV). Nerve latencies (mean±SD) in adults typically range about 2-6 milliseconds, and more typically from about 3.4±0.8 to about 4.0±0.5 milliseconds.

In some embodiments, the system 100 can record a neuromagnetic field outside of the nerves to determine the internal current of the nerves without physical disruption of the nerve membrane. Without being bound by theory, the contribution to the magnetic field from the current inside the membrane is two orders of magnitude larger than that from the external current, and that the contribution from current within the membrane is substantially negligible. Electrical stimulation of the nerve in tandem with measurements of the magnetic compound action fields (“CAFs”) can yield sequential positions of the current dipoles such that the location of the conduction change can be estimated (e.g., via the least-squares method). Visual representation (e.g., via the display 112) using magnetic contour maps can show normal or non-normal neural characteristics (e.g., normal can be equated with a characteristic quadrupolar pattern propagating along the nerve), and therefore indicate which nerves are in a diseases, hyperactive state and suitable targets for neuromodulation.

During magnetic field detection, an array of the electrodes (244, 336) can be positioned in contact with tissue at the interest zone and, optionally, one or more of the electrodes (244, 336) can be activated to inject an electrical stimulus into the tissue. As the nerves in the interest zone fire (either in response to a stimulus or in the absence of it), the nerve generates a magnetic field (e.g., similar to a current carrying wire), and therefore changing magnetic fields are indicative of the nerve nerve-firing rate. The changing magnetic field caused by neural firing can induce a current detected by nearby sensor wire (e.g., the sensor 314) and/or wires associated with the nearby electrodes (244, 336). By measuring this current, the magnetic field strength can be determined. The magnetic fields can optionally be mapped (e.g., on a display 112) to identify the location of nerves and select target nerves (nerves with excessive parasympathetic tone) before neuromodulation therapy to ensure that the desired nerves are treated during neuromodulation therapy. Further, the magnetic field information can be used during or after neuromodulation therapy so that the clinician can monitor changes in nerve firing rate to validate treatment efficacy.

In other embodiments, the neuromagnetic field is measured with a Hall Probe or other suitable device, which can be integrated into the end effector (214, 314) and/or part of a separate device delivered to the interest zone. Alternatively, rather than measuring the voltage in the second wire, the changing magnetic field can be measured in the original wire (i.e. the nerve) using a Hall probe. A current going through the Hall probe will be deflected in the semi-conductor. This will cause a voltage difference between the top and bottom portions, which can be measured. In some aspects of this embodiments, three orthogonal planes are utilized.

In some embodiments, the system 100 can be used to induce electromotive force (“EMF”) in a wire (i.e., a frequency-selective circuit, such as a tunable/LC circuit) that is tunable to resonant frequency of a nerve. In this embodiment, the nerve can be considered to be a current carrying wire, and the firing action potential is a changing voltage. This causes a changing current which, in turn, causes a changing magnetic flux (i.e., the magnetic field that is perpendicular to the wire). Under Faraday's Law of Induction/Faraday's Principle, the changing magnetic flux induces EMF (including a changing voltage) in a nearby sensor wire (e.g., integrated into the end effector (214, 314), the sensor 314, and/or other structure), and the changing voltage can be measured via the system 100.

In further embodiments, the sensor wire (e.g., the sensor 314) is an inductor and, therefore, provides an increase of the magnetic linkage between the nerve (i.e., first wire) and the sensor wire (i.e., second wire), with more turns for increasing effect. (e.g., V2, rms=V1, rms (N2/N1)). Due to the changing magnetic field, a voltage is induced in the sensor wire, and this voltage can be measured and used to estimate current changes in the nerve. Certain materials can be selected to enhance the efficiency of the EMF detection. For example, the sensor wire can include a soft iron core or other high permeability material for the inductor.

During induced EMF detection, the end effector (214, 314) and/or other device including a sensor wire is positioned in contact with tissue at the interest zone and, optionally, one or more of the electrodes (244, 336) can be activated to inject an electrical stimulus into the tissue. As the nerves in the interest zone fire (either in response to a stimulus or in the absence of it), the nerve generates a magnetic field (e.g., similar to a current carrying wire) that induces a current in the sensor wire (e.g., the sensor 314). This information can be used to determine neural location and/or map the nerves (e.g., on a display 112) to identify the location of nerves and select target nerves (nerves with excessive parasympathetic tone) before neuromodulation therapy to ensure that the desired nerves are treated during neuromodulation therapy. EMF information can also be used during or after neuromodulation therapy so that the clinician can monitor changes in nerve firing rate to validate treatment efficacy.

In some embodiments, the system 100 can detect magnetic fields and/or EMF generated at a selected frequency that corresponds to a particular type of nerve. The frequency and, by extension, the associated nerve type of the detected signal can be selected based on an external resonant circuit. Resonance occurs on the external circuit when it is matched to the frequency of the magnetic field of the particular nerve type and that nerve is firing. In manner, the system 100 can be used to locate a particular sub-group/type of nerves.

In some embodiments, the system 100 can include a variable capacitor frequency-selective circuit to identify the location and/or map specific nerves (e.g., parasympathetic nerve, sensory nerve, nerve fiber type, nerve subgroup, etc.). The variable capacitor frequency-selective circuit can be defined by the sensor 314 and/or other feature of the end effector (214, 314). Nerves have different resonant frequencies based on their function and structure. Accordingly, the system 100 can include a tunable LC circuit with a variable capacitor (C) and/or variable inductor (L) that can be selectively tuned to the resonant frequency of desired nerve types. This allows for the detection of neural activity only associated with the selected nerve type and its associated resonant frequency. Tuning can be achieved by moving the core in and out of the inductor. For example, tunable LC circuits can tune the inductor by: (i) changing the number of coils around the core; (ii) changing the cross-sectional area of the coils around the core; (iii) changing the length of the coil; and/or (iv) changing the permeability of the core material (e.g., changing from air to a core material). Systems including such a tunable LC circuit provide a high degree of dissemination and differentiation not only as to the activation of a nerve signal, but also with respect to the nerve type that is activated and the frequency at which the nerve is firing.

### Anatomical Mapping

In various embodiments, the system 100 is further configured to provide minimally-invasive anatomical mapping that uses focused energy current/voltage stimuli from a spatially localized source (e.g., the electrodes (244, 336)) to cause a change in the conductivity of the of the tissue at the interest zone and detect resultant biopotential and/or bioelectrical measurements (e.g., via the electrodes (244, 336)). The current density in the tissue changes in response to changes of voltage applied by the electrodes (244, 336), which creates a change in the electric current that can be measured with the end effector (214, 314) and/or other portions of the system 100. The results of the bioelectrical and/or biopotential measurements can be used to predict or estimate relative absorption profilometry to predict or estimate the tissue structures in the interest zone. More specifically, each cellular construct has unique conductivity and absorption profiles that can be indicative of a type of tissue or structure, such as bone, soft tissue, vessels, nerves, types of nerves, and/or certain neural tissue. For example, different frequencies decay differently through different types of tissue. Accordingly, by detecting the absorption current in a region, the system 100 can determine the underlying structure and, in some instances, to a sub-microscale, cellular level that allows for highly specialized target localization and mapping. This highly specific target identification and mapping enhances the efficacy and efficiency of neuromodulation therapy, while also enhancing the safety profile of the system 100 to reduce collateral effects on non-target structures.

To detect electrical and dielectric tissue properties (e.g., resistance, complex impedance, conductivity, and/or, permittivity as a function of frequency), the electrodes (244, 336) and/or another electrode array is placed on tissue at an interest region, and an internal or external source (e.g., the generator 106) applies stimuli (current/voltage) to the tissue. The electrical properties of the tissue between the source and the receiver electrodes (244, 336) are measured, as well as the current and/or voltage at the individual receiver electrodes (244, 336). These individual measurements can then be converted into an electrical map/image/profile of the tissue and visualized for the user on the display 112 to identify anatomical features of interest and, in certain embodiments, the location of firing nerves. For example, the anatomical mapping can be provided as a color-coded or gray-scale three-dimensional or two-dimensional map showing differing intensities of certain bioelectric properties (e.g., resistance, impedance, etc.), or the information can be processed to map the actual anatomical structures for the clinician. This information can also be used during neuromodulation therapy to monitor treatment progression with respect to the anatomy, and after neuromodulation therapy to validate successful treatment.

In addition, the anatomical mapping provided by the bioelectrical and/or biopotential measurements can be used to track the changes to non-target tissue (e.g., vessels) due to neuromodulation therapy to avoid negative collateral effects. For example, a clinician can identify when the therapy begins to ligate a vessel and/or damage tissue, and modify the therapy to avoid bleeding, detrimental tissue ablation, and/or other negative collateral effects.

Furthermore, the threshold frequency of electric current used to identify specific targets can subsequently be used when applying therapeutic neuromodulation energy. For example, the neuromodulation energy can be applied at the specific threshold frequencies of electric current that are target neuronal-specific and differentiated from other non-targets (e.g., blood vessels, non-target nerves, etc.). Applying ablation energy at the target-specific frequency results in an electric field that creates ionic agitation in the target neural structure, which leads to differentials in osmotic potentials of the targeted neural tissue. These osmotic potential differentials cause dynamic changes in neuronal membronic potentials (resulting from the difference in intra-cellular and extra-cellular fluidic pressure) that lead to vacuolar degeneration of the targeted neural tissue and, eventually, necrosis. Using the highly targeted threshold neuromodulation energy to initiate the degeneration allows the system 100 to deliver therapeutic neuromodulation to the specific target, while surrounding blood vessels and other non-target structures are functionally maintained.

In some embodiments, the system 100 can further be configured to detect bioelectrical properties of tissue by non-invasively recording resistance changes during neuronal depolarization to map neural activity with electrical impedance, resistance, bio-impedance, conductivity, permittivity, and/or other bioelectrical measurements. Without being bound by theory, when a nerve depolarizes, the cell membrane resistance decreases (e.g., by approximately 80×) so that current will pass through open ion channels and into the intracellular space. Otherwise the current remains in the extracellular space. For non-invasive resistance measurements, tissue can be stimulated by applying a current of less than 100 Hz, such as applying a constant current square wave at 1 Hz with an amplitude less than 25% (e.g., 10%) of the threshold for stimulating neuronal activity, and thereby preventing or reducing the likelihood that the current does not cross into the intracellular space or stimulating at 2 Hz. In either case, the resistance and/or complex impedance is recorded by recording the voltage changes. A complex impedance or resistance map or profile of the area can then be generated.

For impedance/conductivity/permittivity detection, the electrodes (244, 336) and/or another electrode array are placed on tissue at an interest region, and an internal or external source (e.g., the generator 106) applies stimuli to the tissue, and the current and/or voltage at the individual receiver electrodes (244, 336) is measured. The stimuli can be applied at different frequencies to isolate different types of nerves. These individual measurements can then be converted into an electrical map/image/profile of the tissue and visualized for the user on the display 112 to identify anatomical features of interest. The neural mapping can also be used during neuromodulation therapy to select specific nerves for therapy, monitor treatment progression with respect to the nerves and other anatomy, and validate successful treatment. In some embodiments of the neural and/or anatomical detection methods described above, the procedure can include comparing the mid-procedure physiological parameter(s) to the baseline physiological parameter(s) and/or other, previously-acquired mid-procedure physiological parameter(s) (within the same energy delivery phase). Such a comparison can be used to analyze state changes in the treated tissue. The mid-procedure physiological parameter(s) may also be compared to one or more predetermined thresholds, for example, to indicate when to stop delivering treatment energy. In some embodiments of the present technology, the measured baseline, mid-, and post-procedure parameters include a complex impedance. In some embodiments of the present technology, the post-procedure physiological parameters are measured after a pre-determined time period to allow the dissipation of the electric field effects (ionic agitation and/or thermal thresholds), thus facilitating accurate assessment of the treatment.

In some embodiments, the anatomical mapping methods described above can be used to differentiate the depth of soft tissues within the nasal mucosa. The depth of mucosa on the turbinates is relatively deep while the depth off the turbinate is relatively shallow and, therefore, identifying the tissue depth in the present technology also identifies positions within the nasal mucosa and where precisely to target. Further, by providing the micro-scale spatial impedance mapping of epithelial tissues as described above, the inherent unique signatures of stratified layers or cellular bodies can be used as identifying the region of interest. For example, different regions have larger or small populations of specific structures, such as submucosal glands, so target regions can be identified via the identification of these structures.

In some embodiments, the system 100 includes additional features that can be used to detect anatomical structures and map anatomical features. For example, the system 100 can include an ultrasound probe for identification of neural tissue and/or other anatomical structures. Higher frequency ultrasound provides higher resolution, but less depth of penetration. Accordingly, the frequency can be varied to achieve the appropriate depth and resolution for neural/anatomical localization. Functional identification may rely on the spatial pulse length (“SPL”) (wavelength multiplied by number of cycles in a pulse). Axial resolution (SPL/2) may also be determined to locate nerves.

In some embodiments, the system 100 can further be configured to emit stimuli with selective parameters that suppress rather than fully stimulate neural activity. For example, in embodiments where the strength-duration relationship for extracellular neural stimulation is selected and controlled, a state exists where the extracellular current can hyperpolarize cells, resulting in suppression rather than stimulation spiking behavior (i.e., a full action potential is not achieved). Both models of ion channels, Hodgkin-Huxley (HH) and Retinol Ganglion Cell (RGC), suggest that it is possible to hyperpolarize cells with appropriately designed burst extracellular stimuli, rather than extending the stimuli. This phenomenon could be used to suppress, rather than stimulate, neural activity during any of the embodiments of neural detection and/or modulation described herein.

In various embodiments, the system 100 could apply the anatomical mapping techniques disclosed herein to locate or detect the targeted vasculature and surrounding anatomy before, during, and/or after treatment.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

What is claimed is:

1. A system for treating a condition within a sino-nasal cavity of a patient, the system comprising:
a treatment device including an end effector comprising one or more electrodes for delivering energy to one or more target sites within the sino-nasal cavity of the patient; and
a console unit operably associated with the treatment device and configured to:
receive, via user input with an interactive interface associated with the console unit, a request for a determination of availability of the one or more electrodes for applying treatment to one or more target sites within a selected one of a left side and a right side of the sino-nasal cavity of the patient;
initiate, in response to the request, an impedance assessment of the one or more electrodes within the selected one of the left and right sides of the sino-nasal cavity; and
output, via the interactive interface, an alert to a user indicating a determined availability of the one or electrodes based on the impedance assessment.

2. The system of claim 1, wherein, upon initiating the impedance assessment, the console unit is configured to:
receive, from the one or more electrodes, impedance measurement data associated with tissue at the one or more target sites within the selected one of the left and right sides of the sino-nasal cavity; and
process the impedance measurement data to calculate a baseline impedance value for each of the one or more electrodes.

3. The system of claim 2, wherein processing the impedance measurement data comprises calculating aggregate impedance values for each of the one or more electrodes or across a set of multiple pairs of the electrodes within a selected one of the left and right sides of the sino-nasal cavity.

4. The system of claim 3, wherein the console unit is configured to process impedance measurement data of all pairs of electrodes of the set within the selected one of the left and right sides of the sino-nasal cavity.

5. The system of claim 2, wherein the determined availability of the one or more pairs of the electrodes is based on a comparison of the calculated baseline impedance value with a predetermined range of baseline impedance values.

6. The system of claim 5, wherein the end effector is multi-segmented and comprises a plurality of support structures that each comprise one or more electrodes, wherein:
at least one of a single, a pair, and a multitude of the plurality of support structures is determined to be available for applying treatment, via one or more associated electrodes, to one or more target sites when the calculated baseline value falls within the predetermined range of baseline impedance values; and
at least one of a single, a pair, and a multitude of the plurality of support structures is determined to be unavailable for applying treatment, via one or more associated electrodes, to one or more target sites when the calculated baseline value falls outside the predetermined range of baseline impedance values.

7. The system of claim 6, wherein:
the console unit permits repositioning of the at least one of the single, the pair, and the multitude of the plurality of support structures determined to be unavailable for applying treatment, via one or more associated electrodes, to one or more target sites when the calculated baseline value falls outside the predetermined range of baseline impedance values; and
the console unit is configured to output at least one of audible alert and visual alert, via the interactive interface, indicating to the user the availability treatment device to provide treatment once successfully repositioned based on a comparison of the calculated baseline impedance value with a predetermined range of baseline impedance values;
wherein the visual alert comprises at least one of text and a first color coding displayed on a graphical user interface (GUI).

8. The system of claim 6, wherein:
the console unit permits transmission of energy from an energy source to one or more electrodes associated with the at least one of the single, the pair, and the multitude of the plurality of support structures determined to be available; and
the console unit prevents transmission of energy from an energy source to one or more electrodes associated with the at least one of the single, the pair, and the multitude of the plurality of support structures determined to be unavailable.

9. The system of claim 8, wherein the energy comprises radiofrequency (RF) energy from an RF generator.

10. The system of claim 8, wherein, upon a determination that at least a minimum required number of pairs of electrodes associated with the at least one of the single, the pair, and the multitude of the plurality of support structures are available, the console unit is configured to output at least one of audible alert and visual alert, via the interactive interface, indicating to the user that the treatment device is ready to provide treatment and further permit transmission of energy from an energy source to one or more electrodes for subsequent delivery of energy to one or more target sites within the selected one of the left and right sides of the sino-nasal cavity.

11. The system of claim 10, wherein the visual alert comprises at least one of text and a first color coding displayed on a graphical user interface (GUI) indicating the availability of one or more pairs of electrodes associated with the at least one of the single, the pair, and the multitude of the plurality of support structures.

12. The system of claim 8, wherein, upon a determination that one or more pairs of electrodes associated with the at least one of the single, the pair, and the multitude of the plurality of support structures is unavailable, the console unit is configured to output at least one of audible alert and visual alert, via the interactive interface, indicating to the user that the treatment device not ready to provide treatment and further prevent transmission of energy from an energy source to one or more electrodes to thereby prevent subsequent delivery of energy to one or more target sites within the selected one of the left and right sides of the sino-nasal cavity.

13. The system of claim 12, wherein the visual alert comprises at least one of text and a second color coding displayed on a graphical user interface (GUI) indicating the unavailability of one or more of the plurality of support structures.

14. The system of claim 6, wherein the multi-segmented end effector comprises a proximal segment that is spaced apart from a distal segment, wherein each of the proximal and distal segments comprises a plurality of support structures that each comprise one or more electrodes.

15. The system of claim 14, wherein at least one of the plurality of support structures comprises a first support structure from the proximal segment and a second support structure from the distal segment.

16. The system of claim 14, wherein electrodes associated with the at least one of the plurality of support structures are configured to deliver energy to the one or more target sites within the selected one of the left and right sides of the sino-nasal cavity of the patient to disrupt multiple neural signals to, and/or result in local hypoxia of, mucus producing and/or mucosal engorgement elements, thereby reducing production of mucus and/or mucosal engorgement within a nose of the patient and reducing or eliminate one or more symptoms associated with at least one of rhinitis, congestion, and rhinorrhea.

17. The system of claim 16, wherein targeted tissue is associated with one or more target sites proximate or inferior to a sphenopalatine foramen, wherein energy is delivered at a level sufficient to therapeutically modulate postganglionic parasympathetic nerves innervating nasal mucosa at foramina and/or microforamina of a palatine bone of the patient and causes multiple points of interruption of neural branches extending through foramina and/or microforamina of palatine bone.

18. The system of claim 16, wherein targeted tissue is associated with one or more target sites proximate or inferior to a sphenopalatine foramen, wherein energy is delivered at a level sufficient to ablate targeted tissue to thereby cause thrombus formation within one or more blood vessels associated with mucus producing and/or mucosal engorgement elements within the nose, wherein the resulting local hypoxia of the mucus producing and/or mucosal engorgement elements results in decreased mucosal engorgement to thereby increase volumetric flow through a nasal passage of the patient.

19. The system of claim 5, wherein the predetermined range of baseline impedance values comprises a low baseline impedance value of approximately 100 ohms and a high baseline impedance value of approximately 1 kohms.

20. The system of claim 19, wherein the predetermined range of baseline impedance values comprises a low baseline impedance value of approximately 400 ohms and a high baseline impedance value of approximately 700 ohms.
